(19) United States
(12) Patent Application Publication
(10) Pub. No.: US 2005/0118690 A1
(43) Pub. Date: Jun. 2, 2005

(54) Love variant regulator molecules

(76) Inventors: Shannon Roberts, Cambridge, MA (US); Amir Sherman, Jerusalem (IL); Joshua Trueheart, Concord, MA (US); G. Todd Milne, Brookline, MA (US)

Correspondence Address: FISH & RICHARDSON PC, 225 FRANKLIN ST, BOSTON, MA, 02110, US

(21) Appl. No.: 10/492,051

(22) PCT Filed: Oct. 9, 2002
(86) PCT No.: PCT/US02/32248

Related U.S. Application Data
(63) Continuation-in-part of application No. 09/974,760, filed on Oct. 9, 2001.
(60) Provisional application No. 60/328,339, filed on Oct. 9, 2001.

(51) Int. Cl. C12P 7/62; C07H 21/04; C12N 1/16; C12N 15/74

(52) U.S. Cl. 435/135; 435/254.3; 435/193; 435/320.1; 435/484; 536/23.2

(57) ABSTRACT

The invention provides variant regulator proteins of secondary metabolite production and nucleic acids encoding said variant regulator proteins. In particular, the invention provides variant regulator molecules of the lovE protein.

### FIELD OF TIE INVENTION

The invention relates to the fields of microbiology and molecular biology. In particular, the invention relates to the field of mycology and the production of secondary metabolites from fungi.

### SUMMARY OF THE RELATED ART

Secondary metabolites are a major source of commercially useful products such as food additives, vitamins, and medicines for the treatment of a wide variety of infections and diseases. By way of example, in 1997 the statin drugs lovastatin, simvastatin, and pravastatin, fugal secondary metabolites used in the treatment of hypercholesteremia, together had US sales of US$7.53 billion (Sutherland et al., Current Opinion In Drug Discovery & Development 4:229-236 (2001)). The cost and availability of these plant, bacterial and fungal metabolites are frequently determined by limitations imposed on production and purification of these compounds from culture. This problem is frequently exacerbated by the fact that these products are generally produced during the stationary phase of bacterial and fugal growth.

A wide variety of methods have been utilized to increase the amount of secondary metabolite produced in culture. Studies have demonstrated the importance of carefully designing the medium in which a fungus is grown to maximize the amount of a secondary metabolite produced (see, e.g., Hajjaj H, et al., Appl. Environ. Microbiol. 67:2596-602 (2001); Lesova, K., et al., J. Basic Microbiol. 40:369-75 (2000)). In addition, the method of culture or fermentation also impacts directly on the amount of secondary metabolite produced. For example, see Robinson, T., et al. (Appl. Microbiol. Biotechnol. 55:284-289 (2001)), which demonstrates the advantages of solid state (substrate) fermentation.

In addition to the manipulation of culture and media conditions, genetic approaches have been taken to increase secondary metabolite production. For example, the production of penicillin is limited by the activity of two enzymes, encoded by the ipnA and acvA genes, both of which are regulated by the pacC protein, a zinc-finger transcription factor. Naturally occurring mutant alleles of the pacC locus are known to possess more transcription-activating activity than the cognate, wild-type allele (see, e.g., Tilburn et al. EMBO J. 14(4):779-790 (1995)). Thus, one genetic approach to increasing secondary metabolite production is to identify and isolate naturally occurring mutant alleles, the expression of which leads to increased secondary metabolite production.

Although many regulators of secondary metabolite production in many organisms are known, not all of the organisms that produce secondary metabolites are amenable to genetic or molecular genetic manipulation. Thus, these systems are not generally useful as a source for the isolation of naturally occurring mutant alleles and are even less useful for the deliberate manipulation of secondary metabolite regulator protein structure with the aim of creating improved regulators of secondary metabolite production.

It would be advantageous to have improved regulators of the biosynthetic enzymes responsible for secondary metabolite production. For example, recent studies suggest increasing usage of statin drugs, e.g., see Waters D. D., Am. J. Cardiol. 88:10F-5F (2001)). Thus, demand for statin drugs is likely to increase substantially. In order to meet the demand for these and other secondary metabolites, new and improved methods for the production of secondary metabolites must be identified.

### BRIEF SUMMARY OF THE INVENTION

The invention provides variant secondary metabolite regulator proteins that enable increased production of secondary metabolites. The invention also provides methods to make these improved regulator proteins. Certain of the variant secondary metabolite regulator proteins have increased ability to stimulate production of secondary metabolites in at least some strains of certain fungal species, e.g., certain strains of Aspergillus terreus or Saccromyces cereviae.

In a first aspect, the invention provides a variant regulator protein of secondary metabolite production with the same greater activity than that of the cognate, wild-type protein in at least some fungal strains In certain embodiments of this aspect of the invention, the regulator protein is a fungal regulator protein.

In an embodiment of the first aspect, the invention provides an improved regulator protein comprising an amino acid sequence coding for a variant lovE protein having at least one specific mutation that gives rise to greater transcription-activating properties of the regulator protein and/or induction of secondary metabolite synthesis in at least some fungal strains.

By way of non-limiting example, certain preferred regulator proteins of this aspect of the invention include at least one of the following mutations (amino acid changes), e.g., in a polypeptide comprising the amino acid sequence of SEQ ID NO:91): (1) a Group 6 amino acid residue (e.g., F) mutated to a Group 2 amino acid residue at position 31, in one embodiment the mutation represented by F31L; :(2) a Group 3 amino acid residue (e.g., Q) mutated to a Group 5 amino acid residue at position 41, in one embodiment the mutation represented by Q41K or Q41R; (3) a Group 4 amino acid residue (e.g., T) mutated to a Group 2 amino acid residue at position 52, in one embodiment the mutation represented by T52I; (4) a Group 4 amino acid residue (e.g., 1) mutated to a Group 3 amino acid residue at position 52, in one embodiment the mutation represented by T52N; (5) a Group 4 amino acid residue (e.g., C) mutated to a Group 5 amino acid residue at position 73, in one embodiment the mutation represented by C73R; (6) a Group 1 amino acid residue (e.g., P) mutated to a Group 4 amino acid residue at position 101, in one embodiment the mutation represented by P101S; (7) a Group 1 amino acid residue mutated to a Group 3 amino acid residue (e.g., P) at position 101, in one embodiment the mutation represented by P101Q; (8) a valine amino acid residue mutated to another Group 2 amino acid residue at position 111, in one embodiment the mutation represented by V111I; (9) a Group 4 amino acid residue (e.g., S) mutated to a Group 2 amino acid residue at position 133, in one embodiment the mutation represented by S133L; (10) a Group 3 amino acid residue (e.g., E) mutated to a Group 2 amino acid residue at position 141, in one embodiment the mutation represented by E141V; (11) a Group 3 amino acid residue (e.g., E) mutated to a Group 5 amino acid residue at position 141, in one embodiment the mutation represented by E141K; (12) a Group 4 amino acid residue (e.g., C) mutated to Group 6 amino acid residue at position 153, in one embodiment the mutation represented by C153Y; (13) a Group 4 amino acid residue (e.g., C) mutated to a Group 5 amino acid residue at position 153, in one embodiment the mutation represented by C153R; (14) a Group 4 amino acid residue (e.g., T) mutated to a Group 1 amino acid residue at position 281, in one embodiment the mutation represented by T281A; (15) a Group 3 amino acid residue (e.g., N) mutated to a Group 2 amino acid residue at position 367, in one embodiment the mutation represented by N367I; (16) a Group 3 amino acid residue (e.g., N) mutated to a Group 6 amino acid residue at position 367, in one embodiment the mutation represented by N367Y; (17) a Group 1 amino acid residue (e.g., P) mutated to Group 4 amino acid residue at position 389, in one embodiment the mutation represented by P389S; and (18) a Group 1 amino acid residue (e.g., P) mutated to a Group 2 amino acid residue at position 389, in one embodiment the mutation represented by P389L.

In some embodiments of the first aspect, the invention provides regulator proteins with at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen of the above described specific mutations.

In other embodiments of the first aspect, the invention provides an isolated lovE variant regulator protein or a polypeptide comprising, consisting of or consisting essentially of an amino acid sequence selected from the group consisting of SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:91, SEQ ID NO:93, and SEQ ID NO:94.

In other embodiments of the first aspect, the invention provides an isolated lovE variant regulator protein or a polypeptide comprising, consisting of or consisting essentially of an amino acid sequence selected from the group consisting of: SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ I) NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:91, with the addition of the amino acid sequence of SEQ ID NO:95 or SEQ ID NO:96 at the amino terminus.

In a second aspect, the invention provides a nucleic acid molecule encoding a lovE regulator of the first aspect of the invention. By way of non-limiting example, the invention provides a nucleic acid molecule encoding the lovE variant regulator protein or a polypeptide comprising, consisting or consisting essentially of an amino acid sequence selected from the group consisting of SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:78, SEQ ID NO:79, SEQ ID NO:81, SEQ ID NO:82, SEQ ID NO:83, SEQ ID NO:84, SEQ ID NO:86, SEQ ID NO:87, SEQ ID NO:88, SEQ ID NO:89, SEQ ID NO:90, SEQ ID NO:91, SEQ ID NO:93, and SEQ ID NO:94. In certain embodiments the polypeptide comprises the amino acid sequence of SEQ ID NO:95 or SEQ ID NO:96 at its amino terminus. In a preferred embodiment of the second aspect, the nucleic acid molecule lacks introns that interrupt the polypeptide coding sequence. Thus, the nucleotide sequence encoding the polypeptide is contiguous.

In a third aspect, the invention provides a method of increasing the activity of a protein that regulates secondary metabolite production comprising: (a) selecting a nucleic acid comprising a polynucleotide encoding a protein regulator of secondary metabolite production; (b) mutating the nucleic acid to create a plurality of nucleic acid molecules encoding variant regulator proteins of secondary metabolite production; and (c) selecting a variant regulator protein with more activity than the cognate, wild-type protein.

In various embodiments of the third aspect, the secondary metabolite is a fungal secondary metabolite. In certain embodiments of the third aspect, the protein regulator of secondary metabolite production is a transcription factor. In certain embodiments of the third aspect, the protein regulator of secondary metabolite production is a transmembrane transporter, protein that mediates secretion, kinase, G-protein, cell surface receptor, GTPase activating protein, guanine nucleotide exchange factor, phosphatase, protease, phosphodiesterase, bacterial protein toxin, importin, RNA-binding protein, SCF complex component, adherin, or protein encoded within a biosynthetic cluster. In certain other embodiments of the third aspect, the variant regulator protein is selected to have more activity in a heterologous cell and/or more activity in a homologous cell than the cognate, wild-type regulator protein. In certain embodiments, the variant regulator protein is selected to have more activity in a heterologous cell and/or more activity in a homologous cell than the cognate, wild-type protein and to cause more secondary metabolite to be produced in a homologous cell and/or a heterologous cell when compared to the cognate, wild-type regulator protein. In a particularly preferred embodiment, the variant regulator protein is a lovE variant regulator protein.

In a fourth aspect, the invention provides a method of increasing production of a secondary metabolite comprising: (a) selecting a nucleic acid comprising a polynucleotide encoding a protein regulator of secondary metabolite production; (b) mutating the nucleic acid to create a plurality of nucleic acid molecules encoding variant regulator proteins of secondary metabolite production; (c) selecting a variant regulator protein with more activity than the cognate, wild-type protein; and (d) expressing the selected variant regulator protein in a cell, thereby increasing production of the secondary metabolite in the cell.

In various embodiments of the fourth aspect, the secondary metabolite is a fungal secondary metabolite. In certain embodiments of the third aspect, the protein regulator of secondary metabolite production is a transcription factor. In certain embodiments of the fourth aspect, the protein regulator of secondary metabolite production is a transmembrane transporter, a protein that mediates secretion, a kinase, a G-protein, a cell surface receptor, a GTPase activating protein, a guanine nucleotide exchange factor, a phosphatase, a protease, a phosphodiesterase, a bacterial protein toxin, an importin, an RNA-binding protein, an SCF complex component, an adherin, or a protein encoded within a biosynthetic cluster. In certain other embodiments of the fourth aspect, the variant regulator protein is selected to have more activity in a heterologous cell and/or more activity in a homologous cell. In certain embodiments, the variant regulator protein is selected to have more activity in a heterologous cell and/or more activity in a homologous cell and to cause more secondary metabolite to be produced in a homologous cell and/or a heterologous cell when compared to the cognate, wild-type regulator protein In a particularly preferred embodiment, the variant regulator protein is a lovE variant regulator protein.

In a fifth aspect, the invention provides an isolated variant regulator protein of secondary metabolite production having increased activity compared to a cognate, wild-type protein, the variant regulator protein made by the process comprising, (a) selecting a nucleic acid comprising a polynucleotide encoding a protein regulator of secondary metabolite production; (b) mutating the nucleic acid to create a plurality of nucleic acid molecules encoding variant regulator proteins of secondary metabolite production; (c) selecting a variant regulator protein with more activity than the cognate, wild-type protein; and (d) recovering the selected variant regulator protein.

In certain embodiments of the fifth aspect, the secondary metabolite is a fungal secondary metabolite. In certain embodiments of the fifth aspect, the protein regulator of secondary metabolite production is a transcription factor. In certain embodiments of the fifth aspect, the protein regulator of secondary metabolite production is a transmembrane transporter, a protein that mediates secretion, a kinase, a G-protein, a cell surface receptor, a GTPase activating protein, a guanine nucleotide exchange factor, a phosphatase, a protease, a phosphodiesterase, a bacterial protein toxin, an importin, an RNA-binding protein, an SCF complex component, an adherin, or a protein encoded within a biosynthetic cluster.

In certain embodiments of the fifth aspect, the variant regulator protein has more activity in a heterologous and/or a homologous cell than the cognate, wild-type protein in at least some fungal strains, e.g., in at least some strains of A. terreus. In certain embodiments of the fourth aspect, the variant regulator protein increases production of a secondary metabolite in a heterologous cell and/or a homologous cell when compared to the cognate, wild-type protein. In a particularly preferred embodiment, the variant regulator protein is a lovE variant regulator protein.

In a sixth aspect, the invention provides a fungus having improved lovastatin production made by the process of transforming a fungal cell with a nucleic acid molecule encoding a lovE variant protein of the first aspect of the invention. In an embodiment thereof, the nucleic acid molecule is selected from a nucleic acid molecule of the second aspect of the invention.

In a seventh aspect, the invention provides an improved process for making lovastatin comprising transforming a fungal cell with a nucleic acid molecule encoding a variant of the lovE protein of the first aspect of the invention. In an embodiment thereof, the fungal cell is transformed with a nucleic acid molecule of the second aspect of the invention.

In a eighth aspect, the invention provides a nucleic acid molecule encoding a lovE protein defined by SEQ ID NO:91. In one embodiment, the nucleic acid molecule comprises a contiguous coding sequence lacking introns encoding a polypeptide comprising SEQ ID NO:91. In an embodiment thereof, the invention provides an isolated lovE nucleic acid molecule defined by SEQ ID NO:92. In an eighth aspect, the invention provides a nucleic acid molecule encoding a lovE protein defined by SEQ ID NO:91. In an embodiment thereof, the invention provides an isolated lovE nucleic acid molecule defined by SEQ ID NO:92.

In a ninth aspect the invention features an isolated polypeptide comprising, consisting of, or consisting essentially of the amino acid sequence of SEQ ID NO:91 having an amino acid change selected from the group consisting of: (a) a Phe changed to a Group 2 amino acid residue at position 31; (b) a Gln changed to a Group 5 amino acid residue at position 41; (c) a Thr changed to a Group 2 amino acid residue at position 52; (d) a Thr changed to a Group 3 amino acid residue at position 52; (e) a Cys changed to a Group 5 amino acid residue at position 73; (f) a Pro changed to a Group 4 amino acid residue at position 101; (g) a Pro changed to a Group 3 amino acid residue at position 101; (h) a Val changed to a Group 2 amino acid residue other than Val at position 111; (i) a Ser changed to a Group 2 amino acid residue at position 133; @) a Glu changed to a Group 2 amino acid residue at position 141; @) a Glu changed to a Group 5 amino acid residue at position 141; (1) a Cys changed to a Group 6 amino acid residue at position 153; (m) a Cys changed to a Group 5 amino acid residue at position 153; (n) a Thr changed to a Group 1 amino acid residue at position 281; (o) a Asn changed to a Group 2 amino acid residue at position 367; (p) a Asn changed to a Group 6 amino acid residue at position 367; (q) a Pro changed to a Group 4 amino acid residue at position 389; and (r) a Pro changed to a Group 2 amino acid residue at position 389.

In various embodiments of the ninth aspect: the polypeptide when expressed in an A. terreus cell harboring a lovF gene increases expression of the lovF gene relative to an otherwise identical cell not expressing the polypeptide; the polypeptide when expressed in a S. cerevisiae harboring a gene under the control of the A. terreus lovF expression control region increases expression of the gene relative to an otherwise identical cell not expressing the polypeptide; the polypeptide has fewer than 11, fewer than 10, fewer than 8, or fewer than 5 amino acid changes; the polypeptide further comprises the amino acid sequence of SEQ ID NO:95 immediately amino terminal to the amino acid of SEQ ID NO:91; the polypeptide further comprises the amino acid sequence of SEQ ID NO:96 immediately amino terminal to the amino acid of SEQ ID NO:91; the isolated polypeptide has the amino acid change F31L, Q41K, Q41R, T52N, C73R, P101S, P101Q, V111I, S133L, E141V, E141K, C153Y, C153R, T281A, N367I, N367Y, P389S, or P389L; and the isolated polypeptide comprises, consists of or consists essentially of an amino acid sequence selected from the group consisting of SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:91, SEQ ID NO:93, and SEQ ID NO:94.

In a tenth aspect the invention features an isolated nucleic acid molecule comprising a nucleotide sequence encoding a polypeptide comprising the amino acid sequence of SEQ ID NO:91 having at least one amino acid change selected from the group consisting of: (a) a Phe changed to a Group 2 amino acid residue at position 31; (0) a Gln changed to a Group 5 amino acid residue at position 41; (c) a Thr changed to a Group 2 amino acid residue at position 52; (d) a Thr changed to a Group 3 amino acid residue at position 52; (e) a Cys changed to a Group 5 amino acid residue at position 73; (f) a Pro changed to a Group 4 amino acid residue at position 101; (g) a Pro changed to a Group 3 amino acid residue at position 101; (h) a Val changed to a Group 2 amino acid residue other than Val at position 111; (i) a Ser changed to a Group 2 amino acid residue at position 133; (j) a Glu changed to a Group 2 amino acid residue at position 141; (k) a Glu changed to a Group 5 amino acid residue at position 141; (l) a Cys changed to a Group 6 amino acid residue at position 153; (m) a Cys changed to a Group 5 amino acid residue at position 153; (n) a Thr changed to a Group 1 amino acid residue at position 281; (o) a Asn changed to a Group 2 amino acid residue at position 367; (p) a Asn changed to a Group 6 amino acid residue at position 367; (q) a Pro changed to a Group 4 amino acid residue at position 389; and (r) a Pro changed to a Group 2 amino acid residue at position 389.

In various embodiments of the tenth aspect: the polypeptide when expressed in an A. terreus cell harboring a lovF gene increases expression of the lovF gene relative to an otherwise identical cell not expressing the polypeptide; the polypeptide when expressed in a S. cerevisiae harboring a gene under the control of the A. terreus lovF expression control region increases expression of the gene relative to an otherwise identical cell not expressing the polypeptide; the polypeptide has fewer than 11, fewer than 10, fewer than 8, or fewer than 5 amino acid changes; the polypeptide further comprises the amino acid sequence of SEQ ID NO:95 immediately amino terminal to the amino acid of SEQ ID NO:91; the polypeptide further comprises the amino acid sequence of SEQ ID NO:96 immediately amino terminal to the amino acid of SEQ ID NO:91; the isolated polypeptide has the amino acid change F31L, Q41K, Q41R, T52N, C73R, P101S, P101Q, V111I, S133L, E141V, E141K, C153Y, C153R, T281A, N367I, N367Y, P389S, or P389L; the isolated polypeptide comprises, consists of, or consists essentially of an amino acid sequence selected from the group consisting of SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:91, SEQ ID NO:93, and SEQ ID NO:94; and the isolated nucleic acid molecule comprises, consists of, or consists essentially of a nucleotide, sequence selected from the group consisting of: SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, and SEQ ID NO:90. In other embodiments of the tenth aspect, the nucleotide sequence encoding the polypeptide is contiguous, i.e., the coding sequence is not interrupted by an intron.

In an eleventh aspect, the invention features a fugal cell containing a nucleic acid molecule encoding any of the forgoing polypeptides.

In a twelfth aspect, the invention feature a fungal cell (e.g., an A. terreus cell) containing any of the forgoing nucleic acid molecules of any of claims 29-56.

In a thirteen aspect, the invention features a method for providing a fungal cell having improved production of a secondary metabolite (e.g., lovastatin), the method comprising transforming the fungal cell with a nucleic acid molecule described above whereby the fungal cell has increased secondary metabolite production compared to an otherwise identical fungal cell that has not been so transformed.

In a fourteenth aspect, the invention features a method for producing a secondary metabolite(e.g., lovastatin), the method comprising providing a fungal cell containing a forgoing nucleic acid molecule, culturing the cell under conditions so as to produce the secondary metabolite, and isolating from the cells a fraction containing the secondary metabolite.

In a fifteenth aspect, the invention features an isolated polypeptide comprising, consisting of, or consisting essentially of the amino acid sequence of SEQ ID NO:91 having an amino acid change selected from the group consisting of: H253R, S341P, R121W, S322G, A83V, T135I, E177G, E197K, T281A, T256A, N466S, C73R, E303K, Q41K, Q41K, P16A, G23S, T9M, Q362E, R21}, S34A, Q80H, A84S, E303D, H374D, A440T, A441V, C445S, P469S, F31L, T409L M971, E113D, D146N, P163S, H458Y, I43V, Q295L, F31L, C159S, E162K, R293L, S311N, L141, E18V, G138C, E338G, V361L, N400S, S174Y, A402T, F31L, P108S, D85N, I143F, M232I, T315I, S382Y, M385K, T461, Q62R, K77R, S323C, V373I, T294I, P310L, G337D, A394V, G436S, T139, V184I, D4E, V87I, D110E, A189T, N276D, T347R, N367I, Q377R, A425T, D131N, R312G, and A429G. In other embodiments, the polypeptide includes at least one such amino acid change.

In various embodiments of the fifteen aspect, the invention features In various embodiments of the ninth aspect: the polypeptide when expressed in an A. terreus cell harboring a lovF gene increases expression of the lovF gene relative to an otherwise identical cell not expressing the polypeptide; the polypeptide when expressed in a S. cerevisiae harboring a gene under the control of the A. terreus lovF expression control region increases expression of the gene relative to an otherwise identical cell not expressing the polypeptide; the polypeptide has fewer than 11, fewer than 10, s fewer than 8, or fewer than 5 amino acid changes; the polypeptide further comprises the amino acid sequence of SEQ ID NO:95 immediately amino terminal to the amino acid of SEQ ID NO:91; the polypeptide further comprises the amino acid sequence of SEQ ID NO:96 immediately amino terminal to the amino acid of SEQ ID NO:91.

### BRIEF DESCRIPTION OF THE DRAWINGS

FIG. 1 is a photographic representation of cells growing on media with and without G418 selection demonstrating lovFp-HIS3p-Neo activation in S. cerevisiae. Controls include MB968 (vector only), MB2478 (lowly expressed wild-type lovE), and MB31644 (highly expressed wild-type lovE). All lovE variants are expressed in an MB968 vector backbone similar to MB2478.

FIG. 2A is a graphic representation of lovFp-CYC1p-lacZ expression in S. cerevisiae strains expressing lovE variant proteins from the clones lovE 1-10.

FIG. 2B is a graphic representation of lovFp-CYC1p-lacZ expression in S. cerevisiae strains expressing lovE variant proteins from the clones lovE 1-10 from a separate transformation than that of FIG. 2A.

FIG. 3 is a graphic presentation of lovFp-CYC1p-lacZ expression in S. cerevisiae strains expressing lovE variant proteins from clones lovE 16-41.

FIG. 4 is a graphic presentation of lovFp-lacZ expression in S. cerevisiae strains expressing lovE variant proteins from clones lovE 1-10.

FIG. 5 is a graphic presentation of lovFp-lacZ expression in S. cerevisiae strains expressing lovE variant proteins from clones lovE 16, 20, 21, 30-34, and 36-41.

FIG. 6 is a graphic presentation of lovastatin culture concentration, as measured by enzyme inhibition assay, from broths of A. terreus cultures expressing lovE variant proteins 1-10 in.

FIG. 7A is a graphic depiction of lovastatin culture concentration, as measured by HPLC analysis, from broths of A. terreus cultures expressing lovE variant proteins 1-10 in M7117.

FIG. 7B is a graphic depiction of lovastatin culture concentration, as measured by HPLC analysis, from broths of A. terreus cultures expressing lovE variant proteins 2, 6, 30, 32, 36, 37, 39, and 41 in MF1 17.

### DETAILED DESCRIPTION OF THE PREFERRED EMBODIMENTS

The invention provides variant secondary metabolite regulator proteins that enable production of secondary metabolites. The invention also provides methods to make these variant regulator proteins. Certain of the variant secondary metabolite regulator proteins have increased ability to stimulate production of secondary metabolites in at least some strains of certain fungal species, e.g., certain strains of Aspergillus terreus or Saccromyces cereviae, compared to the cognate wild-type protein.

In certain embodiments of the aspects of the invention, the invention relates to the biosynthesis and improved production of secondary metabolites. The invention provides variant regulator proteins useful for the production of secondary metabolites, nucleic acid molecules encoding variant regulator proteins, and methods for their production.

As used herein, the terms “fungal” and “fungus” refer generally to eukaryotic, heterotrophic organisms with an absorptive mode of nutrition. Fungi typically contain chitin in their cell walls and exhibit mycelial or yeast-like growth habits (More Gene Manipulations in Fungi, edited by J. W. Bennet and L. L. Lasure, Academic Press Inc. (1991), ISBN 0120886421). More specifically, the terms refer to secondary metabolite producing organisms including, without limitation, Aspergillus sp., Penicillium sp., Acremonium chrysogenum, Yarrowia lipolytica, Nodulisporium sp., Fusarium sp., Monascus sp., Claviceps sp., Trichoderma sp., Tolypocladium sp., Tricotheicium sp., Fusidium sp., Emericellopsis sp., Cephalosporium sp., Cochliobolus sp., Helminthosporium sp., Agaricus brunescens, Ustilago maydis, Neurospora sp., Pestalotiopsis sp. and Phaffia rhodozyma (See, Fungal Physiology, Chapter 9 (Secondary(Special) Metabolism), Griffin, D. H., John Wiley & Sons, Inc.; ISBN: 0471166154).

The term “variant regulator protein” is used herein to refer to any regulatory protein having at least one change or difference in the amino acid sequence of the protein when compared to its cognate, wild-type regulatory protein sequence. The term does not include naturally occurring allelic variations of the cognate, wild-type regulatory protein.

The term “regulator protein” is meant to refer to a protein having a positive or negative function that modifies the production of a secondary metabolite. The function of the protein may be at the level of transcription, e.g., repression or activation, protein synthesis, or transport. The regulator may alter the level of transcription, RNA stability, translation, post-translational modification, or cellular localization of proteins involved in secondary metabolite synthesis and/or transport. The regulator may also have effects on precursor metabolite pools, flux through specific pathways and metabolite resistance.

By way of non-limiting example, certain embodiments of the aspects of the invention relate to a regulator protein that is a protein that contributes and/or promotes transcription of a gene sequence, i.e., a transcription-activating protein. “Transcription-activating” is a term used to refer to characteristics of a protein that promote transcription. As used herein, a transcription-activating protein would include proteins that increase accessibility of the DNA to transcription complexes, for example, by opening or relaxing chromatin structure, proteins that promote the recognition and/or binding of transcription complexes to a target gene sequence, and/or proteins that promote transcription complex movement along the length of the template DNA sequence.

Regulatory proteins of secondary metabolite production and the nucleic acid sequences encoding these are known to those skilled in the art Non-limiting examples of regulatory proteins of secondary metabolite synthesis include: regulator proteins of the aflatoxin/sterigmatocystin biosynthetic cluster (Woloshuk, C. P., et al., Appl, Environ. Microbiol. 60:2408-2414 (1994) and Brown, D. W., et al., Proc Natl Acad Sci U S A. 93:1418-1422 (1996)); regulator proteins of the paxilline biosynthetic cluster (Young, C., et al., Mol, Microbiol. 39:754-764 (2001)); regulator proteins of the cephalosporin and penicillin biosynthetic clusters (Litzka O., et al., Antonie Van Leeuwenhoek 75:95-105 (1999); Schmitt E. Y, and Kuck U., J. Biol. Chem. 275:9348-9357 (2000); MacCabe et al. Mol. Gen. Genet. 250:367-374 (1996); Suarez et al. Mol. Microbiol. 20:529-540 (1996); Lambert et al. Mol. Cell. Biol. 17:3966-3976 (1997); Su et al. Genetics 133:67-77 (1993); regulator proteins of tricothecene synthesis (Trapp S. C., et al., Mol. Gen. Genet. 257:421432 (1998); Brown D. W., et al., Fungal Genet. Biol. 32:121-133 (2001); and Matsumoto G., et al. Biosci. Biotechnol. Biochem. 63:2001-2004 (1999)); and regulator proteins of lovastatin synthesis (Kennedy, J., et al., Science 284:1368-1372 (1999); Hendrickson et al., Chem. Biol. 6:429439 (1999) Tag, A. et al., Mol. Microbiol. 38:658-65 (2000)).

Certain embodiments of the aspects of the invention disclosed herein relate to the lovE regulator protein, a protein which plays a key role in the biosynthesis of lovastatin. More particularly, certain embodiments of the aspects of the invention relate to variant proteins of the lovE regulator protein and methods of making the same. Such proteins are variant with respect to the following A. terreus wild-type lovE sequences (SEQ ID NOS:91 and 92).

The patents and publications cited herein reflect the level of knowledge in the art and are hereby incorporated by reference in their entirety. Any conflict between any teaching of such references and this specification shall be resolved in favor of the latter.

The invention utilizes techniques and methods common to the fields of molecular biology, genetics and microbiology. Useful laboratory references for these types of methodologies are readily available to those skilled in the art. See, for example, Molecular Cloning, A Laboratory Manual, 3^{rd} edition, edited by Sambrook, J., MacCallum, P., and Russell, D. W. (2001), Cold Spring Harbor Laboratory Press (ISBN: 0-879-69576-5); Current Protocols In Molecular Biology, edited by Ausubel, F. M., Brent, R., Kingston, R. E., Moore, D. D., Seidman, J. G., Struhl, K. (1993), John Wiley and Sons, Inc. (ISBN: 0471-306614); PCR Applications: Protocols for Functional Genomics, edited by Innis, M. A., Gelfand, D. I., Sninsky, J. J. (1999), Cold Spring Harbor Press (ISBN: 0-123-72186-5); and Methods In Yeast Genetics, 2000 Edition: A Cold Spring Habor Laboratory Course Manual, by Burke, D., Dawson, D. and Stearns, T., Cold Spring Harbor Press (ISBN: 0-879-69588-9).

TABLE 1
Amino Acid and Nucleic Acid Sequences of Wild-type lovE

| | |
|---|---|
| Wild-type lovE Amino Acid Sequence | |
| maadqgiftnsvtlspvegsrtggtlprrafrrscdrchaqkikctgnkevtgrapcqr | (SEQ ID NO: 91) |
| cqqaglrcvysercpkrklrqsraadlvsadpdpclhmssppvpsqslpldvseshssn | |
| tsrqfldppdsydwswtsigtdeaidtdcwglsqcdggfscqleptlpdlpspfestve | |
| kaplppvssdiaraasaqrelfddlsavsqeleeillavtvewpkqeiwthpigmffna | |
| srrlltvlrqqaqadchqgtldeclrtknlftavhcyilnvriltaiselllsqirrtq | |
| nshmsplegsrsqspsrddtssssghssvdtipffsenlpigelfsyvdplthalfsac | |
| ttlhvgvqllreneitlgvhsaqgiaasismsgepgediartgatnsarceeqpttpaa | |
| rvlfmflsdegafqeaksagsrgrtiaalrrcyedifslarkhkhgmlrdlnnipp | |
| Wild-type lovE DNA Sequence (open reading frame only) | |
| atggctgcagatcaaggtatattcacgaactcggtcactctctcgccagtggagggttc | (SEQ ID NO: 92) |
| acgcaccggtggaacattaccccgccgtgcattccgacgctcttgtgatcggtgtcatg | |
| cacaaaagatcaaatgtactggaaataaggaggttactggccgtgctccctgtcagcgt | |
| tgccagcaggctggacttcgatgcgtctacagtgagcgatgccccaagcgcaagctacg | |
| ccaatccagggcagcggatctcgtctctgctgacccagatccctgcttgcacatgtcct | |
| cgcctccagtgccctcacagagcttgccgctagacgtatccgagtcgcattcctcaaat | |
| acctcccggcaatttcttgatccaccggacagctacgactggtcgtggacctcgattgg | |
| cactgacgaggctattgacactgactgctgggggctgtcccaatgtgatggaggcttca | |
| gctgtcagttagagccaacgctgccggatctaccttcgcccttcgagtctacggttgaa | |
| aaagctccgttgccaccggtatcgagcgacattgctcgtgcggccagtgcgcaacgaga | |
| gcttttcgatgacctgtcggcggtgtcgcaggaactggaagagatccttctggccgtga | |
| cggtagaatggccgaagcaggaaatctggacccatcccatcggaatgtttttcaatgcg | |
| tcacgacggcttcttactgtcctgcgccaacaagcgcaggccgactgccatcaaggcac | |
| actagacgaatgtttacggaccaagaacctctttacggcagtacactgttacatattga | |
| atgtgcggattttgaccgccatatcggagttgctcctgtcgcaaattaggcggacccag | |
| aacagccatatgagcccactggaagggagtcgatcccagtcgccgagcagagacgacac | |
| cagcagcagcagcggccacagcagtgttgacaccatacccttctttagcgagaacctcc | |
| ctattggtgagctgttctcctatgttgaccccctgacacacgccctattctcggcttgc | |
| actacgttacatgttggggtacaattgctgcgtgagaatgagattactctgggagtaca | |
| ctccgcccagggcattgcagcttccatcagcatgagcggggaaccaggcgaggatatag | |
| ccaggacaggggcgaccaattccgcaagatgcgaggagcagccgaccactccagcggct | |
| cgggttttgttcatgttcttgagtgatgaaggggctttccaggaggcaaagtctgctgg | |
| ttcccgaggtcgaaccatcgcagcactgcgacgatgctatgaggatatcttttccctcg | |
| cccgcaaacacaaacatggcatgctcagagacctcaacaatattcctccatga | |

As used herein, the term “secondary metabolite” means a compound, derived from primary metabolites, that is produced by an organism, is not a primary metabolite, is not ethanol or a fusel alcohol, and is not required for growth under standard conditions. Secondary metabolites are derived from intermediates of many pathways of primary metabolism. These pathways include, without imitation, pathways for biosynthesis of amino acids, the shikimic acid pathway for biosynthesis of aromatic amino acids, the polyketide biosynthetic pathway from acetyl coenzyme A (CoA), the mevalonic acid pathway from acetyl CoA, and pathways for biosynthesis of polysaccharides and peptidopolysaccharides. Collectively, secondary metabolism involves all primary pathways of carbon metabolism. Particularly preferred in embodiments of the aspects of the invention are fugal secondary metabolites (See, Fungal Physiology. Chapter 9 (Secondary(Special) Metabolism), Griffin, D. H., John Wiley & Sons, Inc.; ISBN: 0471166154).

“Secondary metabolite” also includes intermediate compounds in the biosynthetic pathway for a secondary metabolite that are dedicated to the pathway for synthesis of the secondary metabolite. “Dedicated to the pathway for synthesis of the secondary metabolite” means that once the intermediate is synthesized by the cell, the cell will not convert the intermediate to a primary metabolite. “Intermediate compounds” also include secondary metabolite intermediate compounds which can be converted to useful compounds by subsequent chemical conversion or subsequent biotransformation. As such, providing improved availability of such intermediate compounds would still lead to improved production of the ultimate useful compound, which itself may be referred to herein as a secondary metabolite. The yeast Saccharomyces cerevisiae is not known to produce secondary metabolites.

The term “primary metabolite” means a natural product that has an obvious role in the functioning of almost all organisms. Primary metabolites include, without limitation, compounds involved in the biosynthesis of lipids, carbohydrates, proteins, and nucleic acids. The term “increasing the yield of the secondary metabolite” means increasing the quantity of the secondary metabolite present in the total fermentation broth per unit volume of fermentation broth or culture.

As used herein, the phrase “modulate production of a secondary metabolite” refers to a positive or negative or desirable change in one or more of the variables or values that affect the process or results of production of the primary or secondary metabolites in a liquid or solid state fungal fermentation. These positive or negative or desirable changes include, without limitation, an increase or decrease in the amount of a primary or secondary metabolite being produced (in absolute terms or in quantity per unit volume of fermentation broth or per unit mass of solid substrate); a decrease in the volume of the broth or the mass/quantity of substrate required for the production of sufficient quantities; a decrease in the cost of raw materials and energy, the time of fermentor or culture run, or the amount of waste that must be processed after a fermentor run; an increase or decrease in the specific production of the desired metabolite (both in total amounts and as a fraction of all metabolites and side products made by the fungus); an increase or decrease in the percent of the produced secondary metabolite that can be recovered from the fermentation broth or culture; and an increase in the resistance of an organism producing a primary or secondary metabolite to possible deleterious effects of contact with the secondary metabolite.

In certain embodiments of aspects of the invention, a secondary metabolite is an anti-bacterial. An “anti-bacterial” is a molecule that has cytocidal or cytostatic activity against some or all bacteria. Preferred anti-bacterials include, without limitation, β-lactams. Preferred β-lactams include, without limitation, penicillins and cephalosporins and biosynthetic intermediates thereof Preferred penicillins and biosynthetic intermediates include, without limitation, isopenicillin N, 6-aminopenicillanic acid (6-APA), penicillin G, penicillin N, and penicillin V. Preferred cephalosporins and biosynthetic intermediates include, without limitation, deacetoxycephalosporin V (DAOC V), deacetoxycephalosporin C (DAOC), deacetylcephalosporin C (DAC), 7-aminodeacetoxycephalosporanic acid (7-ADCA), cephalosporin C, 7-B-(5-carboxy-5-oxopentanamido)-cephalosporanic acid (keto-AD-7ACA), 7-B -(4carboxybutanamido)-cephalosporanic acid (GL,7ACA), and 7-aminocephalosporanic acid (7ACA).

In certain embodiments of aspects of the invention, the secondary metabolite is an anti-hypercholesterolemic or a biosynthetic intermediate thereof An “anti-hypercholesterolemic” is a drug administered to a patient diagnosed with elevated cholesterol levels for the purpose of lowering the cholesterol levels. Preferred anti-hypercholesterolemics include, without limitation, lovastatin, mevastatin, simvastatin, and pravastatin.

According to other embodiments of the invention, a secondary metabolite is an immunosuppressant or a biosynthetic intermediate thereof. An “immunosuppressant” is a molecule that reduces or eliminates an immune response in a host when the host is challenged with an immunogenic molecule, including immunogenic molecules present on transplanted organs, tissues or cells. Preferred immunosuppressants include, without limitation, members of the cyclosporin family and beauverolide L. Preferred cyclosporins include, without limitation, cyclosporin A and cyclosporin C.

In certain embodiments of aspects of the invention, the secondary metabolite is an ergot alkaloid or a biosynthetic intermediate thereof An “ergot alkaloid” is a member of a large family of alkaloid compounds that are most often produced in the sclerotic of fungi of the genus Claviceps. An “alkaloid” is a small molecule that contains nitrogen and has basic pH characteristics. The classes of ergot alkaloids include clavine alkaloids, lysergic acids, lysergic acid amides, and ergot peptide alkaloids. Preferred ergot alkaloids include, without limitation, ergotamine, ergosine, ergocristine, ergocryptine, ergocornine, ergotaminine, ergosinine, ergocristinine, ergocryptinine, ergocorninie, ergonovine, ergometrinine, and ergoclavine.

In certain embodiments of aspects of the invention, the secondary metabolite is an inhibitor of angiogenesis or a biosynthetic intermediate thereof. An “angiogenesis inhibitor” is a molecule that decreases or prevents the formation of new blood vessels. Angiogenesis inhibitors have proven effective in the treatment of several human diseases including, without limitation, cancer, rheumatoid arthritis, and diabetic retinopathy. Preferred inhibitors of angiogenesis include, without limitation, fumagillin and ovalicin.

In certain embodiments of aspects of the invention, the secondary metabolite is a glucan synthase inhibitor or a biosynthetic intermediate thereof A “glucan synthase inhibitor” is a molecule that decreases or inhibits the production of 1,3-β-D-glucan, a structural polymer of fugal cell walls. Glucan synthase inhibitors are a class of antifungal agents. Preferred glucan synthase inhibitors include, without limitation, echinocandin B, pneumocandin B, aculeacin A, and papulacandin.

In certain embodiments of aspects of the invention, the secondary metabolite is a member of the gliotoxin family of compounds or a biosynthetic intermediate thereof. The “gliotoxin family of compounds” are related molecules of the epipolythiodioxopiperazine class. Gliotoxins display diverse biological activities, including, without limitation, antimicrobial, antifungal, antiviral, and immunomodulating activities. Preferred members of the “gliotoxin family of compounds” include, without limitation, gliotoxin and aspirochlorine.

In certain embodiments of aspects of the invention, the secondary metabolite is a fungal toxin or a biosynthetic intermediate thereof. A “fungal toxin” is a compound that causes a pathological condition in a host, either plant or animal. Fungal toxins could be mycotoxins present in food products, toxins produced by phytopathogens, toxins from poisonous mushrooms, or toxins produced by zoopathogens. Preferred fungal toxins include, without limitation, aflatoxins, patulin, zearalenone, cytochalasin, griseofulvin, ergochrome, cercosporin, marticin, xanthocillin, coumarins, tricothecenes, fusidanes, sesterpenes, amatoxins, malformin A, phallotoxins, pentoxin, HC toxin, psilocybin, bufotenine, lysergic acid, sporodesmin, pulcheriminic acid, sordarins, fumonisins, ochratoxin A, and fusaric acid.

With some certain embodiments of aspects of the invention, the secondary metabolite is a modulator of cell surface receptor signaling or a biosynthetic intermediate thereof. The term “cell surface receptor” is as used before. Modulators of cell surface receptor signaling might function by one of several mechanisms including, without limitation, acting as agonists or antagonists, sequestering a molecule that interacts with a receptor such as a ligand, or stabilizing the interaction of a receptor and molecule with which it interacts. Preferred modulators of cell surface signaling include, without limitation, the insulin receptor agonist-L-783,281 and the cholecystokinin receptor antagonist asperlicin.

In certain embodiments of aspects of the invention, the secondary metabolite is a plant growth regulator or a biosynthetic intermediate thereof A “plant growth regulator” is a molecule that controls growth and development of a plant by affecting processes that include, without limitation, division, elongation, and differentiation of cells. Preferred plant growth regulators include, without limitation, cytokinin auxin, gibberellin, abscisic acid, and ethylene.

In certain embodiments of aspects of the invention, the secondary metabolite is a pigment or a biosynthetic intermediate thereof A “pigment” is a substance that imparts a characteristic color. Preferred pigments include, without limitation, melanins and carotenoids.

In certain embodiments of aspects of the invention, the secondary metabolite is an insecticide or a biosynthetic intermediate thereof. An “insecticide” is a molecule that is toxic to insects. Preferred insecticides include, without limitation, nodulisporic acid.

In certain embodiments of aspects of the invention, the secondary metabolite is an anti-neoplastic compound or a biosynthetic intermediate thereof. An “anti-neoplastic” compound is a molecule that prevents or reduces tumor formation. Preferred anti-neoplastic compounds include, without limitation, taxol (paclitaxel) and related taxoids.

The phrase “increased activity” is used herein to refer to a characteristic that results in an augmentation of the inherent negative or positive function of the regulatory protein.

The invention provides variant regulator proteins of secondary metabolite production with increased activity and methods of producing the same. The invention further provides for the identification of specific amino acid residues that are important to the functioning of secondary metabolite regulator proteins. By way of non-limiting example, variant regulator proteins of the secondary metabolite regulator lovE are presented herein.

As known to those skilled in the art, certain substitutions of one amino acid for another may be tolerated at one or more amino acid residues of a wild-type regulator protein absent a change in the structure, activity and/or function of the wild-type protein. Such substitutions are referred to in the art as “conservative” substitutions, and amino acids may be categorized into groups that identify which amino acids may be substituted for another without altering the structure and/or function of the protein.

As used herein, the term “conservative substitution” refers to the exchange of one amino acid for another in the same conservative substitution grouping in a protein sequence. Conservative amino acid substitutions are known in the art and are generally based on the relative similarity of the amino acid side-chain substituents, for example, their hydrophobicity, hydrophilicity, charge, size, and the like. In a preferred embodiment, conservative substitutions typically include substitutions within the following groups: Group 1: glycine, alanine, and proline; Group 2: valine, isoleucine, leucine, and methionine; Group 3: aspartic acid, glutamic acid, asparagine, glutamine; Group 4: serine, threonine, and cysteine; Group 5: lysine, arginine, and histidine; Group 6: phenylalanine, tyrosine, and tryptophan. Each group provides a listing of amino acids that may be substituted in a protein sequence for any one of the other amino acids in that particular group.

As stated supra, there are several criteria used to establish groupings of amino acids for conservative substitution. For example, the importance of the hydropathic amino acid index in conferring interactive biological function on a protein is generally understood in the art (Kyte and Doolittle, Mol. Biol. 157:105-132 (1982). It is known that certain amino acids may be substituted for other amino acids having a similar hydropathic index or score and still retain a similar biological activity. Amino acid hydrophilicity is also used as a criteria for the establishment of conservative amino acid groupings (see, e.g., U.S. Pat. No.4,554,101).

Information relating to the substitution of one amino acid for another is generally known in the art (see, e.g., Introduction to Protein Architecture: The Structural Biology of Proteins, Lesk, A. M., Oxford University Press; ISBN: 0198504748; Introduction to Protein Structures Branden, C.-L., Tooze, J., Karolinska Institute, Stockholm, Sweden (Jan. 15, 1999); and Protein Structure Prediction: Methods and Protocols (Methods in Molecular Biology) Webster, D. M.(Editor), August 2000, Humana Press, ISBN: 0896036375).

In one embodiment of the first aspect, the invention provides an improved regulator protein comprising an amino acid sequence coding for a variant of the lovE protein having at least one specific mutation that gives rise to greater transcription-activating properties of the regulator protein and/or increased lovastatin synthesis.

By way of non-limiting example, certain amino acid residues and mutations thereof in the lovE regulatory protein of A. terreus (SEQ ID NO:91) are identified by the invention described herein. Mutations at residues 31, 41, 52, 73, 101, 111, 133, 141, 153, 281, 367, and 389 of the wild-type lovE protein of A. terreus have been identified as being critical for the improvement of lovE regulator protein function. Those mutations include: F31L, Q41K, Q41R, T52I, T52N, C73R, P101S, P101Q, V111I, S133L, E141V, E141K, C153Y, C153R, T281A, N367I, N367Y, P389S and P389L. Each mutation, therefore, represents a change of one conservative class of amino acids for another. For example, the mutation F31L represents a change from a Group 6 amino acid residue to a Group 2 amino acid residue at position 31 of the wild-type, lovE regulator protein.

Poor transformation efficiency and the lack of efficient selection systems frequently precludes the screening of large numbers of variant regulator proteins of secondary metabolites in the organism from which the regulator protein is isolated. For example, there are currently certain technical obstacles to the successful screening of large numbers of variant regulator proteins in the fungus A. terreus, an organism that produces the secondary metabolite lovastatin.

The invention described herein takes advantage of the genetically tractable and experimentally amenable organism Saccharomyces cerevisiae for screening large numbers of variant regulator proteins of secondary metabolite production. Techniques common to the field of molecular biology are well developed in S. cerevisiae, and large numbers of vectors are available to assist the genetic manipulation and cloning of variant regulator proteins involved in secondary metabolite production. Other genetically tractable organisms could also be used for this purpose.

As used herein, “mutating” is used to refer to the deliberate alteration of at least one nucleotide residue of a wild-type, cognate nucleic acid sequence encoding a regulator protein of secondary metabolite production. A deliberate alteration or change in at least one nucleotide residue of a polynucleotide may be accomplished by any method known in the art. The mutation(s) can be made in vivo or in vitro and can include random, partially random or not random, i.e., directed, mutagenesis techniques.

By way of non-limiting example, in vivo mutagenesis can be done by placing this nucleic acid molecule in a cell with a high mutation frequency, i.e. a mutagenic strain. By way of non-limiting example, Muhlrad et al. (Yeast 8:79-82 (1992)) have developed a rapid method for localized mutagenesis of yeast genes. As a first step, the region of interest of a gene sequence is first amplified in vitro under error-prone polymerase chain reaction (PCR) conditions. Error-prone polymerase chain reaction (PCR) is a method of introducing amino acid changes into proteins. With this technique, mutations are deliberately introduced during the PCR reaction through the, use of error-prone DNA polymerases under specific reaction conditions. With the Muhlrad et al. procedure, the PCR product is then co-transformed with a gapped plasmid containing homology to both ends of the PCR product, resulting in in vivo recombination to repair the gap with the mutagenized DNA.

There are a variety of commercially available kits that may be used to produce mutant nucleic acid molecules by error-prone PCR (see, e.g., GeneMorph™ PCR Mutagenesis Kit (Stratagene, La Jolla, Calif.); and Diversify PCR Random Mutagenesis Kit (13D Biosciences Clontech, Palo Alto, Calif.). Thus, a plurality of variant, i.e., mutated, regulator proteins of secondary metabolite production may be produced using established mutagenesis techniques.

As used herein, the term “activity” refers to a characteristic of the regulator protein that negatively or positively affects the biological system to bring about a modulation in secondary metabolite production. By way of non-limiting example, the activity is the transcription of downstream genes involved in the biosynthetic pathway of the secondary metabolite of choice. Thus, in the present example, the phrase “more activity” refers to the property of a variant regulator protein to bring about more transcription than that effected by the cognate, wild-type regulator protein.

In certain embodiments of the third aspect, the selected variant regulator protein has more activity in a fungal cell than the cognate, wild-type protein. In certain embodiments of the third aspect, the protein regulator of secondary metabolite production is a transcription factor. In certain embodiments of the fourth aspect, the protein regulator of secondary metabolite production is a transmembrane transporter, a protein that mediates secretion, a kinase, a G-protein, a cell surface receptor, a GTPase activating protein, a guanine nucleotide exchange factor, a phosphatase, a protease, a phosphodiesterase, a bacterial protein toxin, an importin, an RNA-binding protein, an SCF complex component, an adherin, or a protein encoded within a biosynthetic cluster. In certain other embodiments of the third aspect, the selected variant regulator protein has more activity in a heterologous cell than the cognate, wild-type protein. In certain embodiments thereof, the heterologous cell is an organism selected from the group consisting of S. cerevisiae, E. coli, A. nidulans, Candida sp., and N. crassa. In yet certain other embodiments of the third aspect, the selected variant regulator protein has more activity in a homologous cell than the cognate, wild-type protein. In certain embodiments thereof, the homologous cell is an organism selected from the group consisting of Aspergillus sp., Penicillium sp., Acremonium chrysogenum, Yarrowia lipolytica, Nodulisporium sp., Fusarium sp., Monascus sp., Claviceps sp., Trichoderma sp., Tolypocladium sp., Tricotheicium sp., Fusidium sp., Emericellopsis sp., Cephalosporium sp., Cochliobolus sp., Helminthosporium sp., Agaricus brunescens, Ustilago maydis, Neurospora sp., Pestalotiopsis sp., and Phaffia rhodozyma.

In certain embodiments of the third aspect, the selected variant regulator protein has more activity in a heterologous cell and a homologous cell than the cognate, wild-type protein. In certain embodiments thereof, the heterologous cell is an organism selected from the group consisting of S. cerevisiae, E. coli, A. nidulans, Candida sp., and N. crassa and the homologous cell is an organism selected from the group consisting of Aspergillus sp., Penicillium sp., Acremonium chrysogenum, Yarrowia lipolytica, Nodulisporium sp., Fusarium sp., Monascus sp., Claviceps sp., Trichoderma sp., Tolypocladium sp., Tricotheicium sp., Fusidium sp., Emericellopsis sp., Cephalosporium sp., Cochliobolus sp., Helminthosporium sp., Agaricus brunescens, Ustilago maydis, Neurospora sp., Pestalotiopsis sp. and Phaffia rhodozyma.

As used herein, the phrase “heterologous cell” refers to a system for gene expression, i.e., an organism for gene expression, that is one other than the organism from which the selected regulator protein of secondary metabolite production has been isolated. Preferred heterologous cells include, but are not limited to, S. cerevisiae, E. coli, A. nidulans, and Candida sp., and N. crassa. Particularly preferred are fugal heterologous cells. In an embodiment of the third aspect, the method comprises: (a) selecting a nucleic acid comprising a polynucleotide encoding a protein regulator of secondary metabolite production; (b) mutating the nucleic acid to create a plurality of nucleic acid molecules encoding variant regulator proteins of secondary metabolite production; and (c) selecting a mutagenized nucleic acid encoding a variant regulator protein with increased activity in a homologous cell than the cognate, wild-type protein.

As used herein, the phrase “homologous cell” refers to a system for gene expression, i.e., an organism for gene expression, that is the organism from which the regulator protein of secondary metabolite production has been isolated. Preferred homologous cells are fugal homologous cells, including, but not limited to, Aspergillus sp., Penicillium sp., Acremonium chrysogenum, Yarrowia lipolytica, Nodulisporium sp., Fusarium sp., Monascus sp., Claviceps sp., Trichoderma sp., Tolypocladium sp., Tricotheicium sp., Fusidium sp., Emericellopsis sp., Cephalosporium sp., Cochliobolus sp., Helminthosporium sp., Agaricus brunescens, Ustilago maydis, Neurospora sp., Pestalotiopsis sp and Phaffia rhodozyma. (See, Fungal Physiology Chapter 9 (Secondary(Special) Metabolism), Griffin, D. H., John Wiley & Sons, Inc.; ISBN: 0471166154).

In certain embodiments of the third aspect, the method further comprises selecting a variant regulator protein that also increases production of a secondary metabolite in a cell when compared to the cognate, wild-type protein. In certain embodiments thereof, the cell is a fungal cell. In certain embodiments thereof, the cell is a heterologous cell, preferably selected from the group consisting of S. cerevisiae, E. coli, A. nidulans, Candida sp., and N crassa.

In certain embodiments thereof, the cell is a homologous cell, preferably selected from the group consisting of Aspergillus sp., Penicillium sp., Acremonium chrysogenum, Yarrowia lipolytica, Nodulisporium sp., Fusarium sp., Monascus sp., Claviceps sp., Trichoderma sp., Tolypocladium sp., Tricotheicium sp., Fusidium sp., Emericellopsis sp., Cephalosporium sp., Cochliobolus sp., Helminthosporium sp., Agaricus brunescens, Ustilago maydis, Neurospora sp., Pestalotiopsis sp., and Phaffia rhodozyma.

Certain embodiments of the aspects of the invention relate to regulator proteins that promote secondary metabolite production by increasing transcription of one or more genes involved with secondary metabolite production. These wild-type sequences may be selected for mutagenesis to create a plurality of variant regulator proteins. The activity of these transcription-activating variant regulator proteins may be determined by measuring the activity of a reporter gene having the appropriate promoter sequences. These tests are done in a homologous and/or a heterologous cell. Certain embodiments of aspects of the invention are directed to fungal regulator proteins with transcription-activating activity that is tested in fungal heterologous and homologous cells.

Reporter genes are useful for isolating transformants expressing improved variant regulator proteins. The reporter genes may be operably linked to a promoter sequence that is normally regulated by the wild-type regulator protein. Reporter genes include, but are not limited to, genes encoding β-galactosidase (lacZ), β-glucoronidase (GUS), β-glucosidase, amylase and invertase, amino acid biosynthetic genes, e.g., the yeast LEU2, HIS3, LYS2, TRP1 genes (or homologous genes from other fungi, such as filamentous fungi, that encode proteins with the similar functional activities), nucleic acid biosynthetic genes, e.g., the yeast URA3 and ADE2 genes (or homologous genes from other fungi, such as filamentous fungi, that encode proteins with the similar functional activities), the mammalian chloramphenicol transacetylase (CAT) gene, or any surface antigen gene for which specific antibodies are available. A reporter gene can also be a neomycin phosphotransferase(neo) gene, which encodes neomycin, kanamycin resistance gene and G418 (geneticin) resistance gene. A reporter gene may encode a protein detectable by luminescence or fluorescence, such as green fluorescent protein (GFP). Reporter genes may additionally or alternatively encode any protein that provides a phenotypic marker, for example, a protein that is necessary for cell growth or viability, or a toxic protein that causes cell death. Alternatively, the reporter gene may encode a protein detectable by a color assay leading to the presence or absence of color.

The choice of reporter gene will depend on the type of cell to be transformed. Preferred reporter genes are those that are operable in fungal cells. It is preferable to have two reporter genes within the cell. One reporter gene, when expressed, provides a growth advantage to transformed cells that are expressing the variant regulator protein. This allows for the isolation of such transformants though selective pressures. The other reporter gene provides a colorimetric marker, such as the lacZ gene and its encoded protein, β-galactosidase. Alternatively, the second reporter provides a fluorescent or luminescent marker, such as green fluorescent protein (GFP).

In a fourth aspect, the invention provides a method of increasing production of a secondary metabolite comprising: (a) selecting a nucleic acid comprising a polynucleotide encoding a protein regulator of secondary metabolite production; (b) mutating the nucleic acid to create a plurality of nucleic acid molecules encoding variant regulator proteins of secondary metabolite production; (c) selecting a variant regulator protein with more activity than the cognate, wild-type protein; and (d) expressing the selected variant regulator protein in a cell, thereby increasing production of the secondary metabolite in the cell.

In certain embodiments of the fourth aspect, the cell is a fungal cell. In certain embodiments of the fourth aspect, the protein regulator of secondary metabolite production is a transcription factor. In certain embodiments of the fourth aspect, the protein regulator of secondary metabolite production is a transmembrane transporter, a protein that mediates secretion, a kinase, a G-protein, a cell surface receptor, a GTPase activating protein, a guanine nucleotide exchange factor, a phosphatase, a protease, a phosphodiesterase, a bacterial protein toxin, an importin, an RNA-binding protein, an SCF complex component, an adherin, or a protein encoded within a biosynthetic cluster. In certain embodiments of the fourth aspect, the cell is a heterologous cell, preferably selected from the group consisting of S. cerevrsiae, E. coli, A. nidulans, Candida sp., and N. crassa. In certain other embodiments of the fourth aspect, the cell is a homologous cell, preferably selected from the group consisting of Aspergillus sp., Penicillium sp., Acremonium chrysogenum, Yarrowia lipolytica, Nodulisporium sp., Fusarium sp., Monascus sp., Claviceps sp., Trichoderma sp., Tolypocladium sp., Tricotheicium sp., Fusidium sp., Emericellopsis sp., Cephalosporium sp., Cochliobolus sp., Helminthosporium sp., Agaricus brunescens, Ustilago maydis, Neurospora sp., Pestalotiopsis sp., and Phaffia rhodozyma.

In certain other embodiments of the fourth aspect, the cell is a heterologous cell and the method further comprises expressing the variant regulator protein in a homologous cell, thereby increasing secondary metabolite production in the homologous cell. In certain embodiments thereof, the heterologous cell is an organism selected from the group consisting of S. cerevisiae, E. coli A. nidulans, Candida sp., and N. crassa and the homologous cell is an organism selected from the group consisting of Aspergillus sp., Penicillium sp., Acremonium chrysogenum, Yarrowia lipolytica, Nodulisporium sp., Fusarium sp., Monascus sp., Claviceps sp., Trichoderma sp., Tolypocladium sp., Tricotheicium sp., Fusidium sp., Emericellopsis sp., Cephalosporium sp., Cochliobolus sp., Helminthosporium sp., Agaricus brunescens, Ustilago maydis, Neurospora sp., Pestalotiopsis sp. and Phaffia rhodozyma.

In a fifth aspect, the invention provides an isolated variant regulator protein of secondary metabolite production having increased activity compared to a cognate, wild-type protein, made by the process comprising: (a) selecting a nucleic acid comprising a polynucleotide encoding a protein regulator of secondary metabolite production; (b) mutating the nucleic acid to create a plurality of nucleic acid molecules encoding variant regulator proteins of secondary metabolite production; (c) selecting a variant regulator protein with more activity than the cognate, wild-type protein; and (d) recovering the selected variant regulator protein.

In certain embodiments of the fifth aspect, the variant regulator protein selected has more activity in a fungal cell. In certain embodiments of the fifth aspect, the protein regulator of secondary metabolite production is a transcription factor. In certain embodiments of the fifth aspect, the protein regulator of secondary metabolite production is a transmembrane transporter, a protein that mediates secretion, a kinase, a G-protein,a cell surface receptor, a GTPase activating protein, a guanine nucleotide exchange factor, a phosphatase, a protease, a phosphodiesterase, a bacterial protein toxin, an importin, an RNA-binding protein, an SCF complex component, an adherin, or a protein encoded within a biosynthetic cluster. In certain embodiments of the fifth aspect, the variant regulator protein selected has more activity in a heterologous cell, preferably selected from the group consisting of S. cerevisiae, E. coli, A. nidulans, Candida sp., Neurospora sp., Pestalotiopsis sp., and N. crassa. In certain embodiments of the fifth aspect, the variant regulator protein selected has more activity in a homologous cell, preferably selected from the group consisting of Aspergillus sp., Penicillium sp., Acremonium chrysogenum, Yarrowia lipolytica, Nodulisporium sp., Fusarium sp., Monascus sp., Claviceps sp., Trichoderma sp., Tolypocladium sp., Tricotheicium sp., Fusidium sp., Emericellopsis sp., Cephalosporium sp., Cochliobolus sp., Helminthosporium sp., Agaricus brunescens, Ustilago maydis, Neurospora sp., Pestalotiopsis sp., and Phaffia rhodozyma.

In certain embodiments of the fifth aspect, the variant regulator protein selected has more activity in a homologous cell and a heterologous cell. In embodiments thereof, the heterologous cell is an organism selected from the group consisting of S. cerevisiae, E. coli, A. nidulans, Candida sp., Neurospora sp., Pestalotiopsis sp., and N. crassa and the homologous cell is an organism selected from the group consisting of Aspergillus sp., Penicillium sp., Acremonium chrysogenum, Yarrowia lipolytica, Nodulisporium sp., Fusarium sp., Monascus sp., Claviceps sp., Trichoderma sp., Tolypocladium sp., Tricotheicium sp., Fusidium sp., Emericellopsis sp., Cephalosporium sp., Cochliobolus sp., Helminthosporium sp., Agaricus brunescens, Ustilago maydis, Neurospora sp., Pestalotiopsis sp., and Phaffia rhodozyma.

In yet another embodiment of the fifth aspect, the variant regulator protein is a variant protein of the lovE protein having at least one of the following mutations: (1) a Group 6 amino acid residue mutated to a Group 2 amino acid residue at position 31, for example, the mutation represented by F31L;(2) a Group 3 amino acid residue mutated to a Group 5 amino acid residue at position 41, for example, the mutation represented by Q41K or Q41R; (3) a Group 4 amino acid residue mutated to a Group 2 amino acid residue at position 52, for example, the mutation represented by T52I; (4) a Group 4 amino acid residue mutated to a Group 3 amino acid residue at position 52, for example, the mutation represented by T52N; (5) a Group 4 amino acid residue mutated to a Group 5 amino acid residue at position 73, for example, the mutation represented by C73R; (6) a Group 1 amino acid residue mutated to a Group 4 amino acid residue at position 101, for example, the mutation represented by P101S; (7) a Group 1 amino acid residue mutated to a Group 3 amino acid residue at position 101, for example, the mutation represented by P101Q; (8) a valine amino acid residue mutated to another Group 2 amino acid residue at position 111, for example, the mutation represented by V111I; (9) a Group 4 amino acid residue mutated to a Group 2 amino acid residue at position 133, for example, the mutation represented by S133L; (10) a Group 3 amino acid residue mutated to a Group 2 amino acid residue at position 141, for example, the mutation represented by E141V; (11) a Group 3 amino acid residue mutated to a Group 5 amino acid residue at position 141, for example, the mutation represented by E141K; (12) a Group 4 amino acid residue mutated to Group 6 amino acid residue at position 153, for example, the mutation represented by C153Y; (13) a Group 4 amino acid residue mutated to a Group 5 amino acid residue at position 153, for example, the mutation represented by C153R; (14) a Group 4 amino acid residue mutated to a Group 1 amino acid residue at position 281, for example, the mutation represented by T281A; (15) a Group 3 amino acid residue mutated to a Group 2 amino acid residue at position 367, for example, the mutation represented by N367I; (16) a Group 3 amino acid residue mutated to a Group 6 amino acid residue at position 367, for example, the mutation represented by N367Y; (17) a Group 1 amino acid residue mutated to Group. 4 amino acid residue at position 389, for example, the mutation represented by P389S; and/or (18) a Group 1 amino acid residue mutated to a Group 2 amino acid residue at position 389, for example, the mutation represented by P389L.

In a sixth aspect, the invention provides a fungus having improved lovastatin production made by the process of transforming a fungal cell with a nucleic acid molecule encoding a variant of the lovE protein of the first aspect of the invention. In an embodiment thereof, the nucleic acid molecule is selected from a nucleic acid molecule of the second aspect of the invention.

In a seventh aspect, the invention provides an improved process for making lovastatin comprising transforming a fungal cell with a nucleic acid molecule encoding a variant of the lovE protein of the first aspect of the invention. In an embodiment thereof, the fugal cell is transformed with a nucleic acid molecule of the second aspect of the invention.

International Patent Application PCT/US99/29583 discloses lovastatin production genes. However, this reference does not provide a mature lovE cDNA sequence. The invention herein remedies the shortcoming of this reference by providing a complete cDNA sequence for the lovE mRNA.

The following examples illustrate the preferred modes of making and practicing the present invention but are not meant to limit the scope of the invention since alternative methods may be utilized to obtain similar results.

### EXAMPLES

### Example 1

### Preparation of Strains and Plasmids

Strain MY2124 was derived from the Sigma 1278b strain background of S. cerevisiae and its complete genotype is as follows: MATα/MATα::LEU2 ura3Δ0/ura3Δ0 leu2Δ0/leu2Δ0 trp1Δ0::hisG/trp1Δ0::hisG his3Δ0::hisG/his3Δ0::hisG ura3Δ0::lovF-HIS3p-neo/ura3Δ0. MY2124 can be constructed by mating S. cerevzsiae strains MY2112 (MATα ura3Δ0 leu2Δ0 trp1Δ0::hisG his3Δ0::hisG ura3Δ0::lovFp-HIS3p-neo) with MY1555 (mat α::LEU2 ura3Δ0 leu2Δ0 trp1Δ0::hisG his3Δ0::hisG) and isolating zygotes. The ura3Δ0::lovFp-HIS3p-neo allele of MY2112 was derived by cotransforming SfiI-linearized plasmid MB2254 with pRS424 (Sikorski and Hieter (1989) Genetics 122:19-27) into MY1413 (MATα leu2Δ0 trp1Δ0::hisG his3Δ0::hisG). Transformants were selected on SC-Trp media and subsequently screened for 5-fluoro-orotic acid resistance to identify those transformants containing the ura3Δ0::lovFp-HIS3p-neo allele. Trp segregants lacking plasmid pRS424 were isolated by growing the strain under non-selective conditions.

The following oligonucleotides were used in the construction of plasmids.

TABLE 2
Oligonucleotides Utilized For LovE Variant Cloning

(SEQ ID NO: 1)

MO664
(5′
GGCCATGGAGGCCGCTAGCTCGAGTCGACGGCCTAGGTGGCCAGCT3′)

(SEQ ID NO: 2)

MO665
(5′
GGCACCTAGGCCGTCGACTCGAGCTAGCGGCCTCCATGGCCGTAC3′)

(SEQ ID NO: 3)

MO666
(5′GGCGGCCGCTCTAGAACTAGTCTCGAGGGTACC3′)

(SEQ ID NO: 4)

MO667
(5′GGTACCCTCGAGACTAGTTCTAGAGCGGCCGCC3′)

(SEQ ID NO: 5)

MO1794
(5′CACAGCGGCCGCTCAACCTTCCCATTGGGGC3′)

(SEQ ID NO: 6)

MO1793
(5′CACCACTAGTACGCGGGCTGATTCGAC3′)

(SEQ ID NO: 7)

MO1785
(5′CACCACTAGTTATACATTATATAAAGTAATGTG3′)

(SEQ ID NO: 8)

MO1786
(5′CACAGGATCCGTCATCTTTGCCTTCGTTTATC3′)

(SEQ ID NO: 9)

MO195
(5′CGCGGATCCTATTGAACAAGATGGATTGCAC3′)

(SEQ ID NO: 10)

MO196
(5′CCGGAATTCAGAAGAACTCGTCAAGAAG3′)

(SEQ ID NO: 11)

MO841
(5′ACAAAAAAGCAGGCTCCACAATGGCTGCGATCAAGGTAT3′)

(SEQ ID NO: 12)

MO842
(5′ACAAGAAAGCTGGGTTCATGGAGGAATATTGTTGA3′)

(SEQ ID NO: 13)

MO2278
(5′GGGGATCCAATCGAGGTCCACGACCAGT3′)

(SEQ ID NO: 14)

MO343
(5′GGGGACAAGTTTGTACAAAAAAGCAGGCT3′)

(SEQ ID NO: 15)

MO2273
(5′GGGGATCCGCCAATGGTCCCGTTCAAAC3′)

(SEQ ID NO: 16)

MO2274
(5′ACAAGAAAGCTGGGTTCACAGAATGTTTAGCTCAA3′)

(SEQ ID NO: 17)

MO344
(5′GGGGACCACTTTGTACAAGAAAGCTGGGT3′)

(SEQ ID NO: 18)

MO2624
(5′GCGATGCCCCAAGCGCAAGCTACGCCAATCCAGGG3′)

(SEQ ID NO: 19)

MO2654
(5′CGTCGCGCCATTCGCCATTCAGGCTGCGCAACTGT3′)

(SEQ ID NO: 20)

MO2680
(5′GGACCTTTGCAGCATAAATTACTATACTTCT3′)

(SEQ ID NO: 21)

MO2686
(5′GGCGCGTCCATTCGCCATTCAGGCTGCGCAACTGT3′)

(SEQ ID NO: 22)

MO2681
(5′TAAAACTCTTGTTTTCTTCTTTTCTCTAAAT3′)

(SEQ ID NO: 23)

MO2700
(5′CAGTGAGCGCGCGTAATACGACTCACTATAGGGCGA3′)

(SEQ ID NO: 24)

MO2701
(5′ATACTTCTATAGACACACAAACACAAATACACACAC3′)

(SEQ ID NO: 25)

MO107
(5′CGCGGATCCCGTCGTTTTACAAC3′)

(SEQ ID NO: 26)

MO197
(5′CCCAAGCTTATTATTTTTGACACCAGACCAA3′)

(SEQ ID NO: 27)

MO1293
(5′GGAAGATCTAGCATCGTGGCCAATTTCTTCTAGTTT3′)

(SEQ ID NO: 28)

MO1294
(5′ATAAGAATGCGGCCGCTCAACCTTCCCATTGGGGCGTTTGC3′)

(SEQ ID NO: 29)

MO1787
(5′CACAGGATCCAGCATTATTAATTTAGTGTGTGTATTT3′)

(SEQ ID NO: 30)

MO1788
(5′CACCACTAGTCTCGAGCAGATCCGCCAG3′)

(SEQ ID NO: 31)

MO1793
(5′CACCACTAGTACGCGGGCTGATTCGAC3′)

(SEQ ID NO: 32)

MO1794
(5′CACAGCGGCCGCTCAACCTTCCCATTGGGGC3′)

(SEQ ID NO: 33)

MO511
(5′GGCCATCGATACAAGTTTGTACAAAAAAGCTGAAC3′)

(SEQ ID NO: 34)

MO540
(5′GGCGCCCTATTACACCACTTTGTACGAAAGC3′)

(SEQ ID NO: 35)

MO1985
(5′CACACGTCTCCGGCCTCAACCTTCCCATTGGGGCG3′)

(SEQ ID NO: 36)

MO1986
(5′CACACAGATCTCGTGGCCAATTTCTTCTAGTTTGA3′)

(SEQ ID NO: 37)

MO1992
(5′CACACGGATCCAGATGTTACGTCCTGTAGAAACCCC3′)

(SEQ ID NO: 38)

MO1993
(5′CACAGCGGCCGCTTCATTGTTTGCCTCCCTGCTG3′)

(SEQ ID NO: 39)

MO316
(5′GCGGCCGCGGCGCCCGGCCCATGTCAACAAGAAT3′)

(SEQ ID NO: 40)

MO318
(5′CCGCGGCCGAGTGGAGATGTGGAGT3′)

Plasmid MB2254 contains the lovFp-HIS3p-neo reporter gene flanked by URA3 sequence. First primers MO664 (SEQ ID NO:1) and MO665 (SEQ ID NO:2) were annealed and inserted into the KpnI-SacI sites of plasmid pBluescript II KS (Stratagene,). The resulting vector, MB1038, contains a SalI site in the polylinker. Next, the SpeI-XhoI fragment from pJL164 (Brachmann et al. Yeast 14:115-132. (1998)) containing a deletion of the URA3 gene with additional flanking sequences was inserted into the NheI-SalI sites of MB1038 to create MB1053. Primers MO666 (SEQ ID NO:3) and MO667 (SEQ ID NO:4) that contain multiple restriction sites (NotI, XbaI, SpeI, XhoI and KpnI) were then annealed together and ligated into the SmaI site of MB1053 to create MB1054. Next, the following four fragments were combined in MB 1054 to obtain plasmid MB2254. The lovF promoter from A. terreus genomic DNA was PCR amplified with MO1794 (SEQ ID NO:5) and MO1793 (SEQ ID NO:6) and inserted into MB1054 on a NotI-SpeI fragment. The HIS3 basal promoter from pRS403 (Sikorski and Hieter, Genetics 122:19-27 (1989)) was PCR amplified with primers MO1785 (SEQ ID NO:7) and MO1786 (SEQ ID NO:8) and inserted into MB1054 on a SpeI-BamHI fragment. Finally, the neo gene (PCR amplified with MO195 (BamHI) (SEQ ID NO:) and MO196 (EcoRI) (SEQ ID NO:10) from plasmid pYX11 (Xiao and Weaver, Nucl. Acids Res. 25:2985-2991 (1997)) and CYC1 terminator sequences (XhoI-KpnI fragment from pRS426-GAL-S (Mumberg, et al., Nucl. Acids. Res. 22:5767-5768 (1994)) were first combined in pRS416.(Sikorski and Hieter, Genetics 122:19-27 (1989)) and then cut out with BamHI-KpnI and inserted into MB 1054 to create MB2254.

The lovFp-HIS3p-neo reporter in MY2124 can confer resistance to the drug geneticin (G418). It was empirically determined that MY2124 (untransformed or transformed with parental plasmids MB2478 (CYC1-lovE/CEN) or MB2848 (CYC1-lovE/At274/CEN) was unable to grow on YPD media supplemented with 100 μg/ml G418. Plasmid MB2478 contains the CYC1 promoter operationally linked to the entire A. terreus lovE open reading frame. The CYC1 promoter is a relatively weak promoter and thus the lovE ORF in MB2478 was expressed at low levels. MB2478 was the parental vector plasmid for creating full length lovE variants. Plasmid MB2848 contains the CYC1 promoter operationally linked to a chimeric open reading frame consisting of the A. terreus lovE DNA binding domain fused to the carboxy-terminal portion of the At274 gene (U.S. Ser. No. 60/257,431, filed Dec. 22, 2000).

MB2848 was used to create lovE variants in which the DNA binding domain was not mutated. Both MB2478 and MB2848 contain yeast CEN and autonomously replicating sequences and both are maintained at 1-2 copies per cell. In contrast to strains transformed with MB2478 or MB2848, strains transformed with plasmid MB1644 (TEF1-lovE/2 micron) were able to grow on G418-supplemented YPD media. The lovE gene of MB1644 is under control of the constitutively strong S. cerevisiae TEF1 promoter. MB1644 contains a 2-micron origin for high-copy replication in yeast. An objective of these studies was to identify lovE variants which when expressed at low levels could confer G418 resistance similar to the highly expressed wild-type lovE molecule of MB1644. S. cerevisiae expression vectors used in these studies were constructed as follows.

MB968 is a low copy S. cerevisiae URA3 based expression vector. MB968 was created by inserting the EcoRV fragment (containing the destination cassette) from gateway pEZC7201 (Invitrogen™, Carlsbad, Calif.) into XhoI/SalI (filled in with Klenow) linearized pRS416 CYC1 (Mumberg, et al., Gene 156:119-122 (1995)).

MB1644 and MB2478 are URA3-based S. cerevisiae expression plasmids that contain the wild-type lovE gene. They are both derivatives of MB1199. MB1199 was created by using primers MO841 (SEQ ID NO:11) and MO842 (SEQ ID NO:12) to amplify the lovE ORF from A. terreus cDNA. Gateway (Invitrogen™, Carlsbad, Calif.) Cloning Technology (U.S. Pat. No. 5,888,732) was used to clone the lovE PCR fragment into the gateway entry vector pDONR206 (Invitrogen™, Carlsbad, Calif.) to create MB1199. Similarly, Gateway Cloning Technology was used to transfer the lovE ORF from MB1199 into MB968 to create MB2478 and into MB969 (U.S. Ser. No. 60/198,335, filed Apr. 18, 2000) to create MB1644.

MB2848 is a derivative of MB968 that contains a lovE-AT274 chimera. The lovE portion of MB2848 was derived by using oligos MO841 (SEQ ID NO:11) and MO2278 (SEQ ID NO:13) to PCR amplify the lovE DNA binding domain from A. terreus cDNA. A second round of PCR was performed with primers MO343 (SEQ ID NO:14) and MO2278 to add appropriate Gateway Cloning Technology compatible sequences. The At274 portion of MB2848 can be derived by using primers MO2273 (SEQ ID NO:1 5) and MO2274 (SEQ ID NO:16) to PCR amplify the carboxy-terminal domain of At274 from A. terreus cDNA. A second round of PCR was performed with primers MO344 (SEQ ID NO:17) and MO2273 to add appropriate Gateway Cloning Technology compatible sequences. The lovE and At274 PCR products were cut with BamHI and purified over a QIAquick PCR purification kit (Qiagen, Valencia, Calif.) according to manufacturer's instructions. Finally, the products were mixed 3-4 hours in a standard ligation reaction and used in Gateway entry and destination reactions to create MB2848.

Gateway cloning technology was used to clone the lovE variants of interest into plasmid MB1419 which is a filamentous fungal expression vector. The MB1419 fungal selection marker is the A. nidulans GPD promoter controlling the ble gene from S. hindustanus. The transgene is controlled by the A. nidulans PGK promoter. A. terreus strain MF117 is a derivative of A. terreus strain ATCC 20542.

### Example 2

### PCR Mutagenesis of the lovE DNA Binding Domain

The zinc finger DNA binding domain of lovE is encoded by nucleotides 100-201 (SEQ ID NO:92). Oligos MO2624 (SEQ ID NO:18) and MO2654 (SEQ ID NO:19) were used to PCR amplify a lovE containing fragment from plasmid MB2478. The 1.7 kb product contains nucleotides 212-1410 of lovE and ˜500 bp of flanking vector sequence. Two rounds of standard PCR (1.5 mM MgCl₂) were performed with Amplitaq DNA polymerase (Applied Biosystems, Foster City, Calif.) according to the manufacturer's instructions.

Plasmid MB2848 was cut with KpnI-BamHI to release a 1.1 kb fragment containing the At274 portion of the lovE-At274 chimeric open reading frame. The remaining 5.5 kb vector sequence retains the lovE DNA binding domain.

### Example 3

### PCR Mutagenesis of the lovE Open Reading Frame

lovE open reading frame insert was prepared according to the following procedure. Oligo pairs MO2680 (SEQ ID NO:20)/MO2686 (SEQ ID NO:21), MO2681 (SEQ ID NO:22)/MO2686, and MO2700 (SEQ ID NO:23)/MO2701 (SEQ ID NO:24) were used to PCR amplify the entire lovE open reading frame from plasmid MB2478. The PCR products differ in the amount of 5′ and 3′ vector sequence flanking the lovE open reading frame.

PCR was performed using a GeneMorph PCR mutagenesis kit (Stratagene, La Jolla, Calif.) according to manufacturer's instructions to achieve medium and high range mutation frequencies.

Plasmid MB2478 was cut with Asp718/XbaI to release a 1.7 kb fragment. The remaining 5.0 kb vector sequence completely lacks lovE ORF sequence.

### Example 4

### Transformation and Selection for G418R Isolates

All PCR products were purified using a QIAquick PCR purification kit (Qiagen) according to manufacturer's instructions. All vectors were gel purified using a QIAquick gel extraction kit (Qiagen) according to manufacturer's instructions.

The mutagenesis strategy of Muhlrad et al. (Yeast 8:79-82 (1992))was used which involves cotransforming a mutated PCR product and gapped plasmids into S. cerevisiae, and then screening for in vivo recombinants having the desired phenotype).

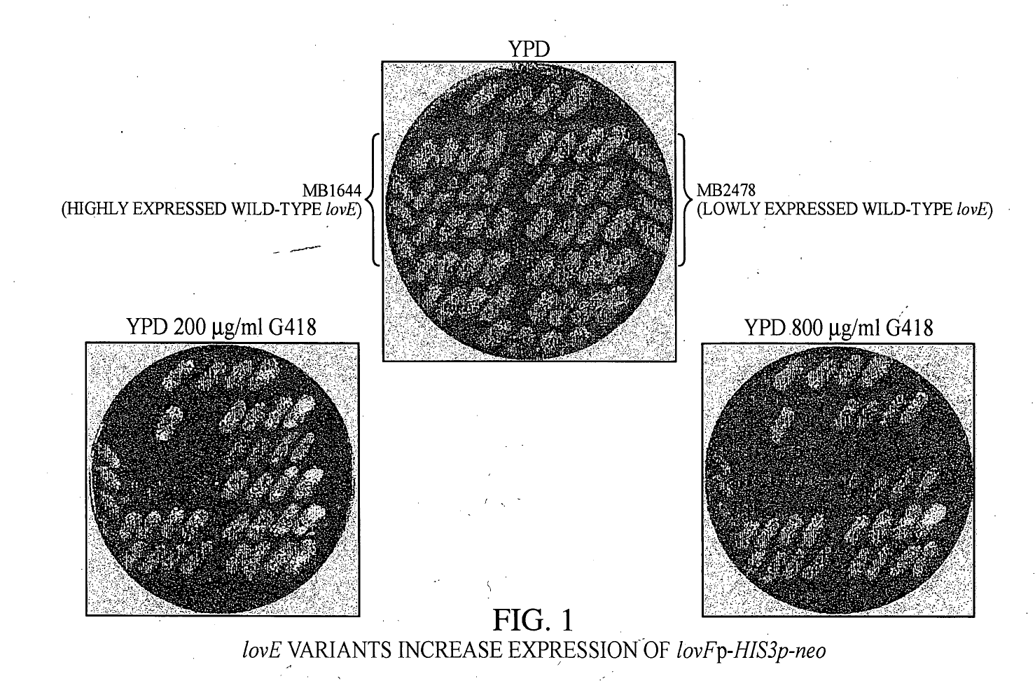

Transformation of Saccharomyces cerevisiae was accomplished by the lithium acetate/single-stranded carrier DNA/polyethylene glycol (LiAc/ss-DNA/PEG) protocol (Woods R. A. and Gietz R. D. Methods Mol. Biol. 177:85-97 (2001)) with a 1:5 molar ratio of vector:insert DNA to generate >55,000 in vivo recombinant transformants on SC-Ura plates. Transformants were transferred by replica printing to YPD plates containing 100 μg/ml G418 and allowed to grow for 2-4 days at 30° C. (FIG. 1).

Drug resistant clones were confirmed in secondary assays including growth on G418 concentrations up to 2000 μg/ml. The plasmid-dependence of the phenotype was determined by observing the re-appearance of drug sensitivity correlating with loss of the library plasmid. lovE variant plasmids were recovered from promising candidates (Hoffman and Winston (1986) Gene 57:267). More than 70 lovE variants were identified and definitively characterized by DNA sequence and/or restriction digestion analysis.

Table 3 summarizes the G418 resistance phenotype and sequence analysis of 26 of these variants.

TABLE 3
Variant LovE Mutations

| | lovFp- | MO aligos used | Amino | Amino | Amino | Amino | Amino | Amino | Amino | Amino | Amino | Amino | Amino |
|---|---|---|---|---|---|---|---|---|---|---|---|---|---|
| | neo | for random | Acid | Acid | Acid | Acid | Acid | Acid | Acid | Acid | Acid | Acid | Acid |
| lovE | Mediated | PCR | Change | Change | Change | Change | Change | Change | Change | Change | Change | Change | Change |
| allele | G418R | mutagenesis | 1 | 2 | 3 | 4 | 5 | 6 | 7 | 8 | 9 | 10 | 11 |
| 1 | −/+ | 2624/2654 | H253R | S341P | | | | | | | | | |
| 2 | +/− | 2624/2654 | R121W | S133L | S322G | | | | | | | | |
| 3 | +++ | 2624/2654 | C73R | A83V | T135I | | | | | | | | |
| 4 | ++ | 2624/2654 | C73R | E177G | | | | | | | | | |
| 5 | ++ | 2624/2654 | C73R | | | | | | | | | | |
| 6 | +/− | 2624/2654 | C153Y | E197K | T281A | | | | | | | | |
| 7 | + | 2624/2654 | C73R | T256A | N466S | | | | | | | | |
| 8 | +++ | 2624/2654 | C73R | E141V | | | | | | | | | |
| 9 | ++ | 2624/2654 | C73R | E303K | | | | | | | | | |
| 10 | +++ | 2624/2654 | Q41K | | | | | | | | | | |
| 16 | +++ | 2680/2686 | Q41K | P16A | G23S | T9M | Q362E | | | | | | |
| 19 | +/− | 2700/2701 | R21H | S34A | Q80H | A84S | E303D | H374D | A440T | A441V | C445S | P469S | |
| 20 | + | 2700/2701 | F31L | T409I | | | | | | | | | |
| 21 | +++ | 2700/2701 | F31L | M97I | E113D | D146N | P163S | N367I | H458Y | | | | |
| 30 | +/− | 2681/2686 | I43Y | Q295L | | | | | | | | | |
| 31 | ++ | 2680/2686 | F31L | P101S | C153R | C159S | E162K | R293L | S311N | | | | |
| 32 | ++ | 2680/2686 | L14I | E18V | G138C | E338G | V361L | P389S | N400S | | | | |
| 33 | ++ | 2680/2686 | Q41R | S174Y | A402T | | | | | | | | |
| 34 | ++ | 2680/2686 | F31L | T52I | P101Q | P108S | V111I | | | | | | |
| 36 | +/− | 2700/2701 | D85N | I143F | M232I | T315I | S382Y | M385K | | | | | |
| 37 | ++ | 2700/2701 | T46I | Q62R | K77R | S323C | N367Y | V373I | | | | | |
| 38 | −/+ | 2700/2701 | Q41R | T294I | P310L | G337D | P389L | A394V | G436S | | | | |
| 39 | + | 2680/2686 | T52N | V111I | T139 | V184I | T281A | | | | | | |
| 40 | +++ | 2680/2686 | Q41R | D4E | V87I | D110E | E141K | A189T | N276D | T347R | N367I | Q377R | A425T |
| 41 | −/+ | 2680/2686 | D131N | S133L | R312G | A429G | | | | | | | |
| wild- | − | N/A | N/A | | | | | | | | | | |
| type | | | | | | | | | | | | | |

Table 4 summarizes amino acid substitutions that were isolated multiple times, suggesting that they are particularly important for improving lovE variant activity on lovFp-HIS3p-neo expression.

TABLE 4
lovE Mutations Isolated Multiple Times

| Amino Acid | Number of Times | |
|---|---|---|
| Change | Isolated in lovE 1-41 | lovE variant |
| F31L | 4 | 20, 21, 31, 34 |
| Q41K | 2* | 10, 16 |
| Q41R | 3* | 33, 38, 40 |
| T521/T52N | 1 each | 34, 39 |
| C73R | 6* | 3, 4, 5, 7, 8, 9 |
| P101S/P101Q | 1 each | 31, 34 |
| V111I | 2 | 34, 39 |
| S133L | 2 | 2, 41 |
| E141V, E141K | 1 each | 8, 40 |
| C153Y/C153R | 1 each | 6, 31 |
| T281A | 2 | 6, 39 |
| N367I/N367Y | 2/1 | 21, 40, 37 |
| P389S/P389L | 1 each | 32, 38 |

*allele was isolated in additional lovE variants that were not fully sequenced

### Example 5

### Increased lovF-lacZ Expression in S. cerevisiae

In order to quantify the increase in lovF expression, β-galactosidase activity was measured in lovE variant transformed S. cerevisiae strains that also harbored lovFp-lacZ reporter derivative plasmids. lovF-lacZ reporter derivative plasmids were constructed as follows.

Plasmid MB1918 contains the lovFp-lacZ reporter gene. It can be derived from pRS424 (Sikorski and Hieter (1989) Genetics 122:19-27). First, primers MO107 (SEQ ID NO:25) and MO197 (SEQ ID NO:26) are used to PCR amplify the lacZ gene from Yep355 (Myers, et al., Gene 45:299-310 (1986)). This lacZ-containing fragment was inserted into the BamHI-HindIII sites of pRS416 (Sikorski and Hieter, Genetics 122:19-27 (1989)). This same lacZ fragment can be cut out of the resulting vector with KpnI-NotI and inserted into the same sites of pRS424 to create pRS424-lacZ. Primers MO1293 (SEQ ID NO:27) and MO1294 (SEQ ID NO:28) are used to PCR amplify a 2.09 kb fragment of the lovF promoter from A. terreus genomic DNA. The lovF promoter fragment was then cut with NotI-BgIII and inserted into NotI-BamHI linearized pRS424-lacZ.

Plasmid MB2114 contains the lovFp-CYC1p-lacZ reporter gene. It can be derived from pRS424-lacZ (see MB1918 plasmid construction). Primers MO1787 (SEQ ID NO:29) and MO1788 (SEQ ID NO:30) are used to amplify the 264 bp basal CYC1 element from pRS415 CYC1 (Mumberg, et al., Gene 156:119-122 (1995)). This 264 bp fragment was inserted upstream of the pRS424-lacZ derivative which has been digested with SpeI-BamHI. Finally, the lovE promoter from MB1918 was PCR amplified with MO1793 (SEQ ID NO:31) and MO1794 (SEQ ID NO:32) and inserted into the NotI-SpeI sites to create MB2114.

Yeast strains utilized in this study include strains MY2145 and MY2159, which are both derived from the S. cerevisiae sigma 1278b strain background; the genotypes are both strains are as follows: MATa ura3Δ0 leu2Δ0 his3Δ::hisG trp1Δ0::hisG. MY2145 and MY2159 contain the lovFp-lacZ reporter plasmids MB2114 and MB1918, respectively.

MY2124 transformed with individual lovE variant plasmids was mated to S. cerevisiae strains MY2154 and MY2159. Diploids were selected on SC-UraTrp media Multiple diploids from each individual mating were assayed for lovFp-lacZ expression using 96 well format β-galactosidase assays. For β-galactosidase assays, cells were transferred from transformation plates to 96-well microtiter plates containing 200 μl Z buffer. 12 strains were transferred simultaneously using a 12-channel multi-pipettor to scoop cells from transformation plates. Duplicate samples were prepared for all assays. OD₆₀₀ readings were taken on samples in Z buffer. These values were used to normalize for equal cell number in all assays. After determining OD₆₀₀, 150 μl of each sample in Z buffer was transferred onto a Millipore Multiscreen Assay System (Nitrocellulose Immobilon N.C.), filtered, and then washed by filtering 200 μl Z buffer. 100 μl Z buffer with βME and detergents was then added to each well, as was 20 μl 4 mg/ml ONPG. Reactions were incubated at 30° C., stopped with 50 βl 1 M Na₂CO₃, filtered into a polystyrene 96-well assay plate, and OD₄₂₀ was determined for each assay well. β-galactosidase units were determined using the Miller formula (O.D. 420×1000)/(OD600*minutes*volume in mL). Z buffer is made by dissolving the following in 1 L of water (16.1 g Na₂HPO₄-7H₂O, 5.5 g NaH₂PO₄-H₂O, 0.75 g KCl and 0.246 g MgSO₄-7H₂O). Z buffer with detergents and βME is made as follows: 9.8 ml Z buffer, 100 μl 20 mg/ml CTAB, 100 μl 10 mg/ml sodium deoxycholate, and 69 μl βME Control plasmids utilized in these studies included MB968, MB2478 and MB1644.

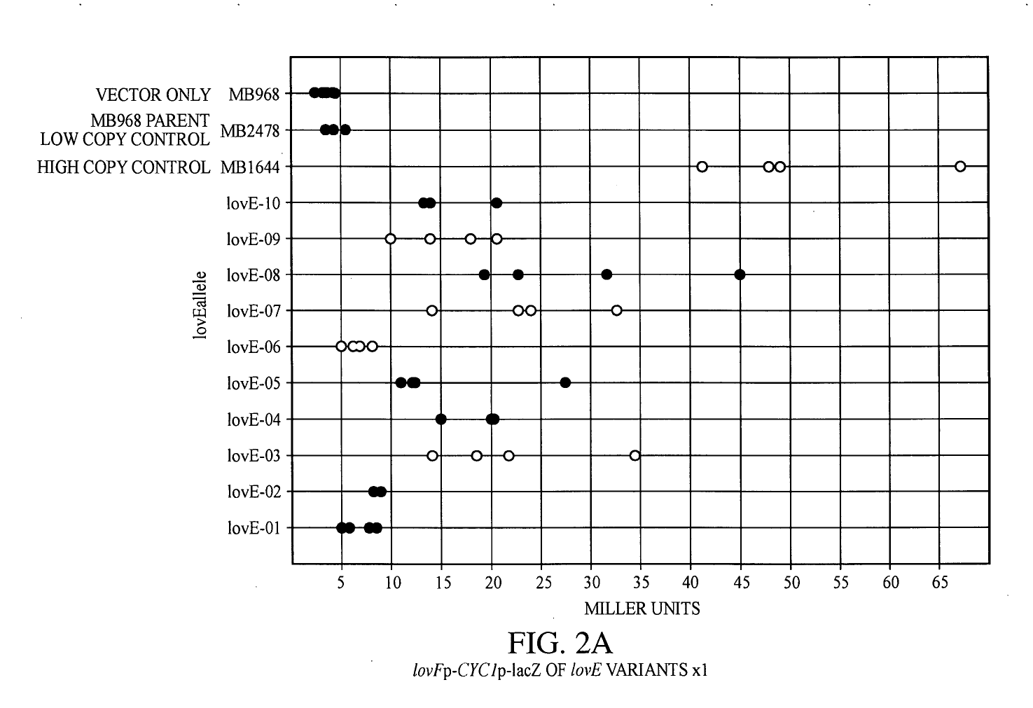

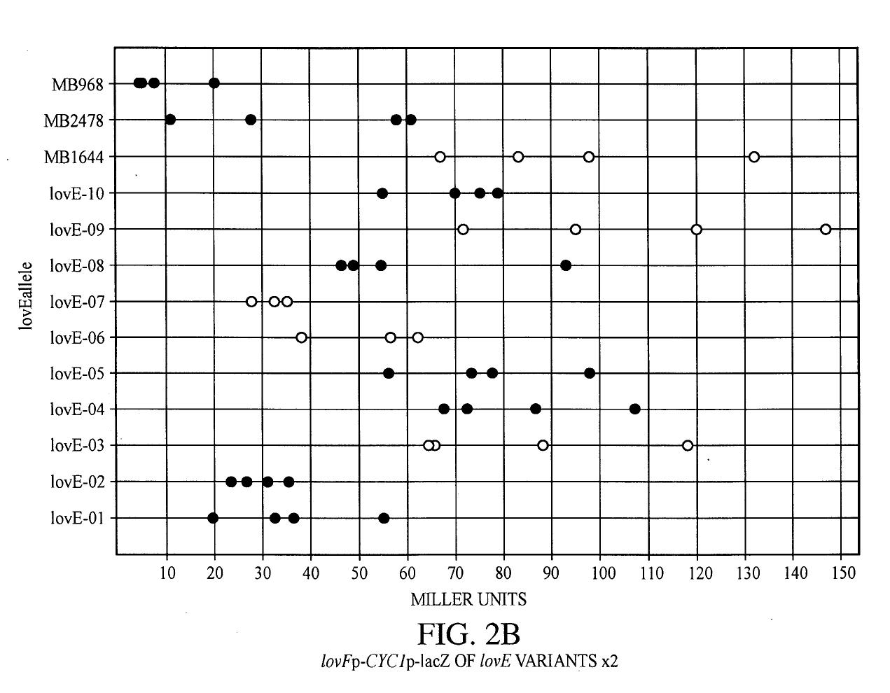

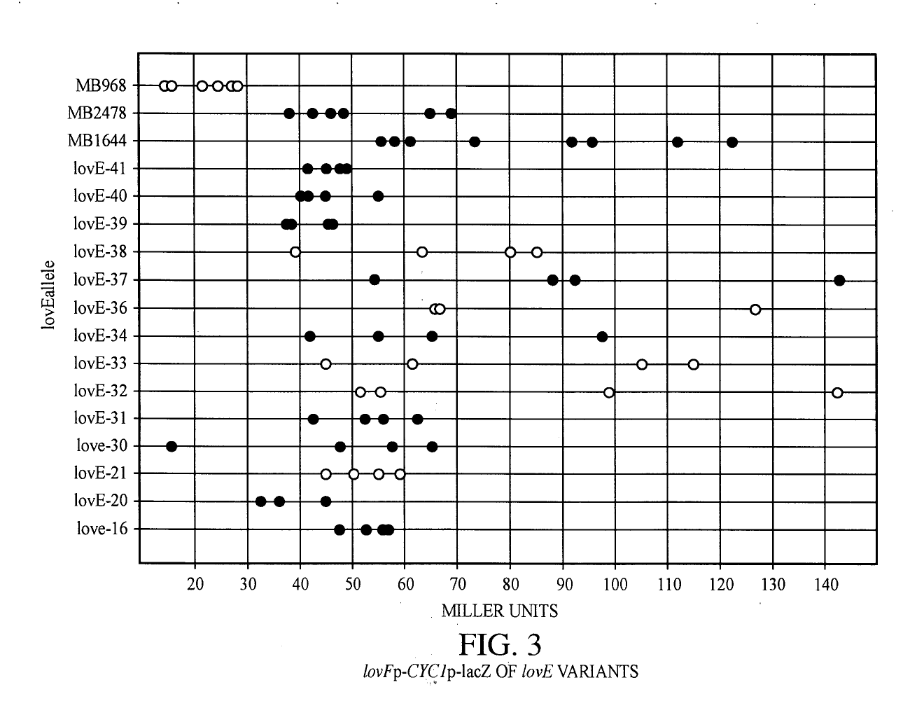

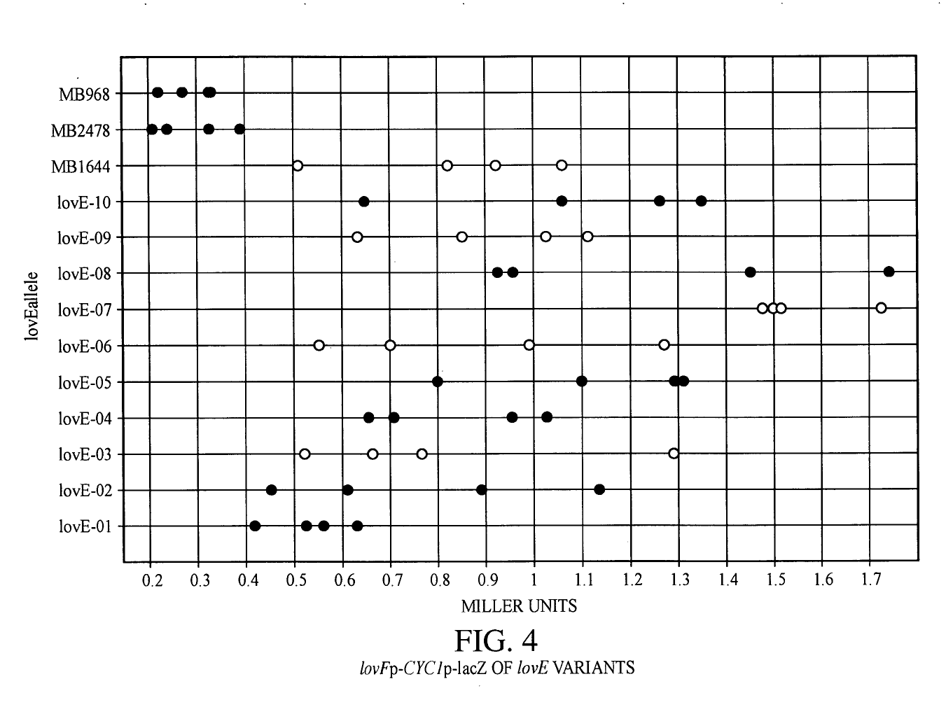

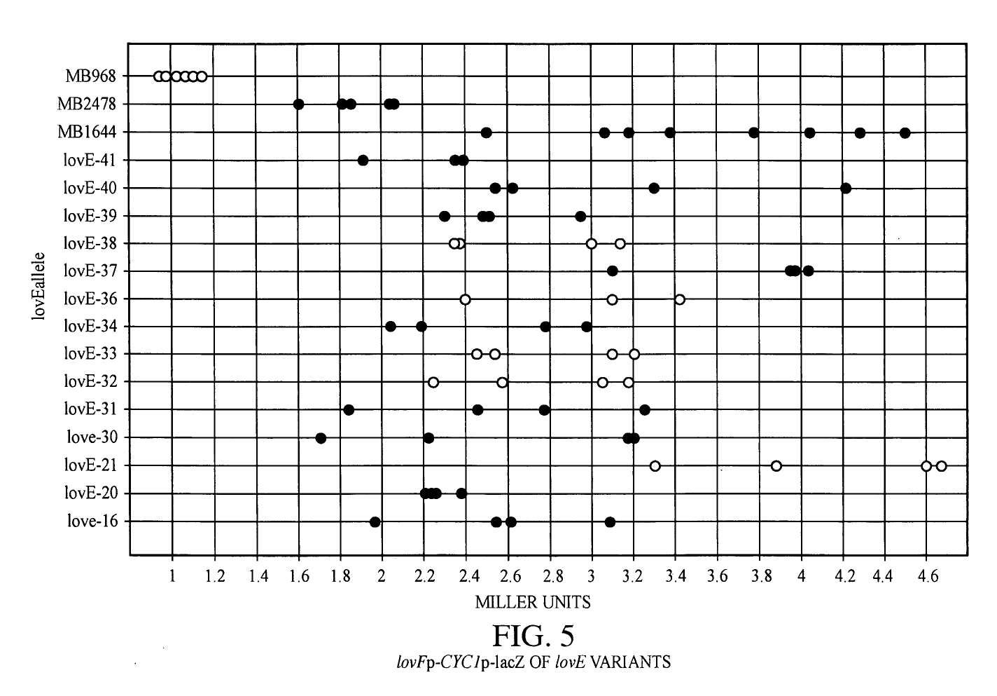

Results of these studies are presented in FIGS. 2-5, demonstrating increased transcription-activating properties of the lovE variants disclosed herein.

### Example 6

### Secondary Metabolite Production

Transformation of filamentous fungi was performed according to the following procedure. Protoplasts were generated by inoculating rich media with spores. Spores were allowed to germinate for about 20 hrs or until germ tubes were between 5 and 10 spore lengths. The germlings were centrifuged and washed twice with sterile distilled water and once with 1 M magnesium sulfate. Germlings were then resuspended in 1M magnesium sulfate containing approximately 2 mg/ml of Novozyme. Tubes were then incubated at 30° C. shaking at 80 RPM for about 2 hrs or until most of the hyphae were digested and protoplasts were abundant. Protoplasts were filtered through one layer of Miracloth. At least one volume of STC was added and protoplasts were centrifuged. Protoplasts were washed twice with STC. Protoplasts then were resuspended in 1 ml STC and counted in a hemacytometer. A final concentration of approximately 5×10⁷ protoplasts/ml were frozen in a 9:1:0.1 solution of STC, SPTC and DMSO in a Nalgene Cryo cooler at −80° C. (cools −1° C./min).

Solutions for transformation were as follows: STC (0.8 M Sorbitol, 25 mM Tris-HCl pH 7.5, 25 mM CaCl₂) and SPTC (0.8 M Sorbitol, 40% PEG 4000, 25 mM Tris-HCl pH 8, 50 mM CaCl₂). Transformation was accomplished according to the following protocol. 1-5 μg of DNA comprising a lovE variant according to the invention in a fungal expression vector was placed in a 50 ml Falcon tube. 100 μl of previously frozen protoplasts were added to the DNA, gently mixed, and then incubated on ice for 30 min. 15 μl of SPTC was added, followed by mixing by tapping and incubation at RT for 15 min. 500 μl SPTC was added and mixed well by tapping and rolling, then incubated at RT for 15 min. 25 mls of regeneration minimal medium was added, mixed well and poured on plates containing 25 mls of regeneration minimal medium with 2× the concentration of selection drug.

Transformation plates were incubated at 26° C. for 5-6 days or until colonies started to appear. Regeneration minimal medium contains trace elements, salts, 25 mM sodium nitrate, 0.8 M Sucrose, and 1% agarose at pH 6.5. The selection drug that was used successfully with A. terreus is phleomycin, a broad-spectrum glycopeptide antibiotic. Transformants were picked onto new plates with a toothpick (if the fungus was sporulating) or with sterile forceps (if the fungus did not sporulate). Purification plates contained minimal medium (same as regeneration minimal medium but containing 2 % instead of 0.8 M sucrose) and 1× drug concentration. Picked transformants were incubated at 26° C. for 5-6 days.

Transformants were grown in production media for secondary metabolite production. Briefly, for A. terreus and lovastatin production, spores were used as the inoculum. Spores were obtained from the purification plate by using a wooden inoculation stick. The medium was RPM containing corn steep liquor, sodium nitrate, potassium phosphate, magnesium sulfate, sodium chloride, P2000 (Dow chemical), trace elements and lactose or glucose as carbon source. The medium was pH 6.5. Flasks were incubated at 26° C. with shaking at 225 RPM. For static 96-well cultures, the same medium was used and the spores were obtained from the purification plate with a wooden toothpick. 96-well plates were incubated, without shaking at 26° C.

Sampling was done after after 5 days for lovastatin. For shake flask experiments 1-1.5 mls of supernatant was placed into 96-well plates, which were centrifuged and supernatants transferred to new 96-well plates. Samples were frozen at −80° C. for storage or for later assays.

Cultures that were grown standing in a 96-well plate were centrifuged and the supernatant was transferred to a new 96 well plate. Samples were frozen at −80° C.

### Example 7

### Measurement of Secondary Metabolite Production

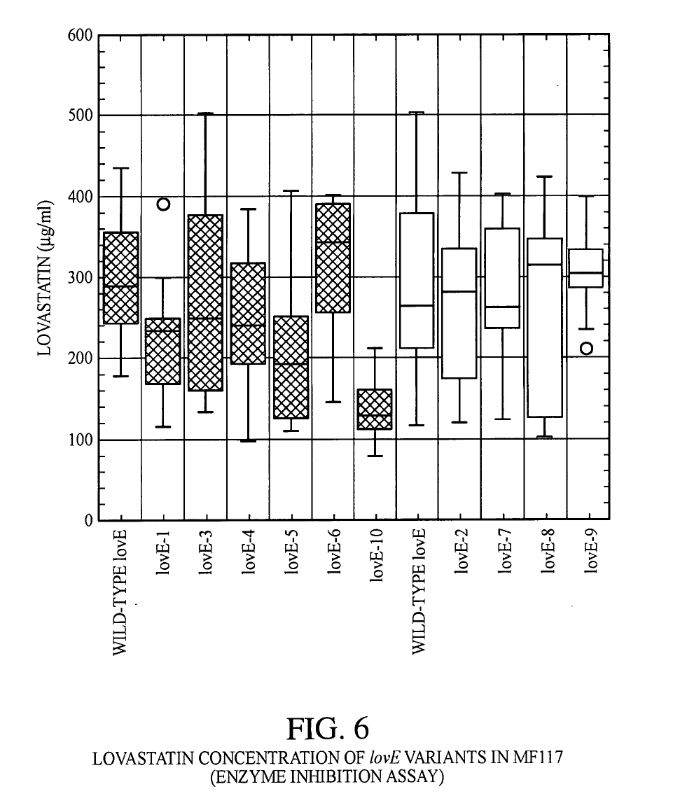

The concentration of the secondary metabolite lovastatin was determined by enzyme inhibition assay FIG. 6). Briefly, 10 μL of sample was removed and diluted 1:100 in H₂O. 10 μl of this diluted broth was assayed in a reaction (200 μL total) containing 1 mM HMGCoA, 1 mM NADPH, 0.005 mM DTT and 5 μl (His)₆HMGR The disappearance of absorbance at 340 nm was observed over time. This represents the disappearance of NADPH, and lovastatin inhibits this reaction.

The initial velocities were calculated for the reactions containing samples, adjusted for dilution, and compared to reactions containing lovastatin standards to determine levels of metabolite produced. (His)₆HMGR was expressed in Saccharomyces cerevisiae and purified with a nickel column.

The results from ten individual transformants for each allele are shown in standard box plot format in FIG. 6. Lovastatin concentration from the corresponding wild-type lovE control is shown in matching fill pattern For example, lovE alleles 2, 7, 8 and 9 were all transformed and assayed at the same time as the non-hatched wild-type control. The horizontal line in each individual box represents the median.

Lovastatin concentration was also determined by high pressure liquid chromatography (HPLC). Briefly, 100 μL of broth sample was removed and diluted 1:10 into 70% H₂O-30% acetonitrile (900 μl). This mixture was spun down to pellet debris at 13000 RPM for 5 minutes. 900 μl of this diluted broth was transferred to a vial and the sample was analyzed by HPLC. 10 μl were injected into a Waters HPLC system (996 photo-diode array detector, 600 E pump controller and 717 autosampler) equipped with a YMC-Pack ODS column (Aq-302-3, 150×4.6 mm ID, S-3 μM pore size) and eluted with isocratic 40% aqueous acetic acid (0.7%)60% acetonitrile for 8 minutes. Lovastatin was detected at 238 nm to have a retention time of 6.5 minutes and was quantified using a calibration curve created from pure lovastatin samples.

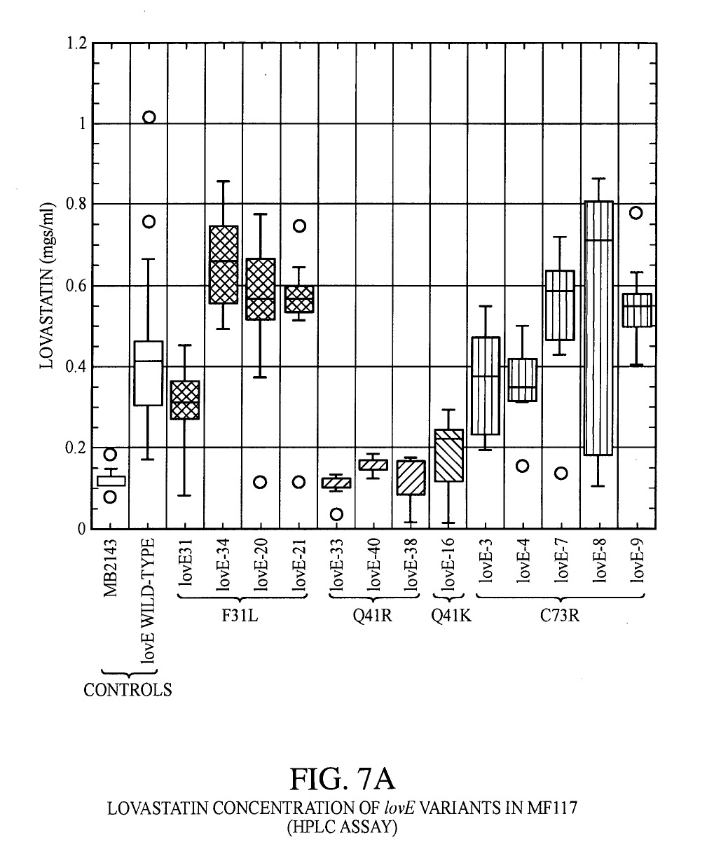

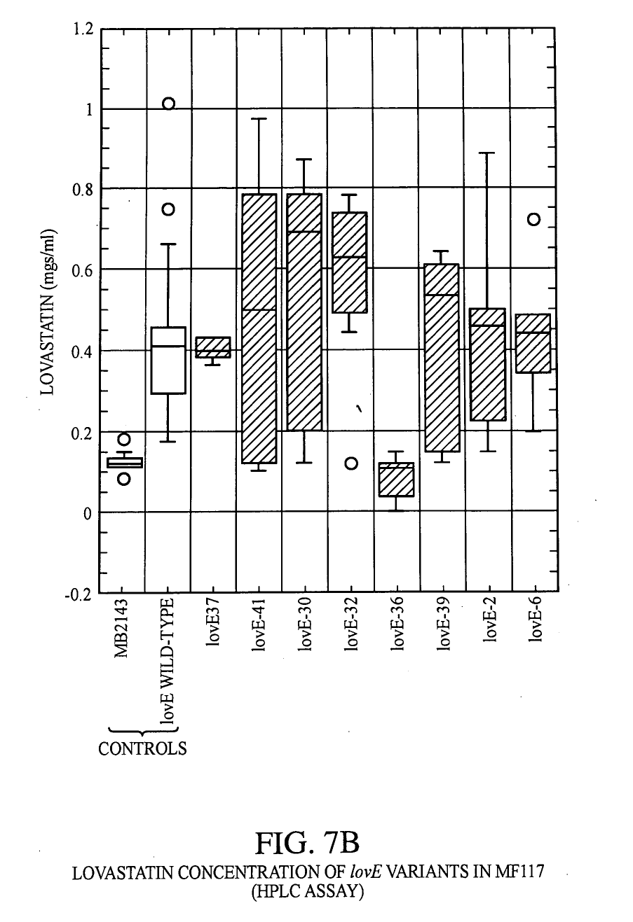

The results from ten individual transformants for each lovE variant are shown in standard box plot format in FIGS. 7A and 7B. Thirty individual wild-type lovE transformants and ten individual MB2143 negative control transformants were tested. Identical controls are plotted in FIGS. 7A and 7B.

PCR analysis of A. terreus transformants demonstrates that greater than fifty percent of the transformants contain the transgene. Variability in levels of transgene expression can presumably be influenced by integration site and copy number. lovE variants containing identical amino acid substitutions are labeled.

The amino acid and nucleic acid sequences of lovE variant sequences are presented in Table 5 and Table 6, respectively.

### Example 8

### Isolation of Additional Forms of love

An A. terreeus cDNA was screened to identify sequences that increase expression of a lovF reporter gene in A. terreus. This analysis led to the identification of two cDNAs that could encode lovE variants having additional amino aicds at their amino terminus compared to the lovE of SEQ ID NO:91. One variant, at242, has the amino sequence mtqdtaqyrga (SEQ ID NO:95) preceding the sequence of SEQ ID NO:91. The entire amino acid sequence of at242 (SEQ ID NO:93) is shown in Table 7. The other variant, at258, has the amino sequence mlmtqdtaqyrga (SEQ ID NO:96) preceding the sequence of SEQ ID NO:91. The entire amino acid sequence of at258 (SEQ ID NO:94) is shown in Table 7. Thus, both variants appear to encode forms of lovE that is longer than the lovE of SEQ ID NO:91. The various amino acid changes present in the various love variants of Table 5 can be introduced into the at242 or at 258 to generate additional forms of lovE.

TABLE 5
Amino Acid Sequences of Variants of the lovE Gene

| | |
|---|---|
| lovE-1 | |
| maadqgiftnsvtlspvegsrtggtlprrafrrscdrchaqkikctgnkevtgrapcqr | (SEQ ID NO: 41) |
| cqqaglrcvysercpkrklrqsraadlvsadpdpclhmssppvpsqslpldvseshssn | |
| tsrqfldppdsydwswtsigtdeaidtdcwglsqcdggfscqleptlpdlpspfestve | |
| kaplppvssdiaraasaqrelfddlsavsqeleeillavtvewpkqeiwthpigmffna | |
| srrlltvlrqqaqadcrqgtldeclrtknlftavhcyilnvriltaiselllsqirrtq | |
| nshmsplegsrsqspsrddtssssghssvdtipffsenlpigelfpyvdplthalfsac | |
| ttlhvgvqllreneitlgvhsaqgiaasismsgepgediartgatnsarceeqpttpaa | |
| rvlfmflsdegafqeaksagsrgrtiaalrrcyedifslarkhkhgmlrdlnnipp | |
| lovE-2 | |
| maadqgiftnsvtlspvegsrtggtlprrafrrscdrchaqkikctgnkevtgrapcqr | (SEQ ID NO: 42) |
| cqqaglrcvysercpkrklrqsraadlvsadpdpclhmssppvpsqslpldvseshssn | |
| tswqfldppdsydwlwtsigtdeaidtdcwglsqcdggfscqleptlpdlpspfestve | |
| kaplppvssdiaraasaqrelfddlsavsqeleeillavtvewpkqeiwthpigmffna | |
| srrlltvlrqqaqadchqgtldeclrtknlftavhcyilnvriltaiselllsqirrtq | |
| nshmsplegsrsqspsrddtssssghgsvdtipffsenlpigelfsyvdplthalfsac | |
| ttlhvgvqllreneitlgvhsaqgiaasismsgepgediartgatnsarceeqpttpaa | |
| rvlfmflsdegafqeaksagsrgrtiaalrrcyedifslarkhkhgmlrdlnnipp | |
| lovE-3 | |
| maadqgiftnsvtlspvegsrtggtlprrafrrscdrchaqkikctgnkevtgrapcqr | (SEQ ID NO: 43) |
| cqqaglrcvyserrpkrklrqsrvadlvsadpdpclhmssppvpsqslpldvseshssn | |
| tsrqfldppdsydwswisigtdeaidtdcwglsqcdggfscqleptlpdlpspfestve | |
| kaplppvssdiaraasaqrelfddlsavsqeleeillavtvewpkqeiwthpigmffna | |
| srrlltvlrqqaqadchqgtldeclrtknlftavhcyilnvriltaiselllsqirrtq | |
| nshmsplegsrsqspsrddtssssghssvdtipffsenlpigelfsyvdplthalfsac | |
| ttlhvgvqllreneitlgvhsaqgiaasismsgepgediartgatnsarceeqpttpaa | |
| rvlfmflsdegafqeaksagsrgrtiaalrrcyedifslarkhkhgmlrdlnnipp | |
| lovE-4 | |
| maadqgiftnsvtlspvegsrtggtlprrafrrscdrchaqkikctgnkevtgrapcqr | (SEQ ID NO: 44) |
| cqqaglrcvyserrpkrklrqsraadlvsadpdpclhmssppvpsqslpldvseshssn | |
| tsrqfldppdsydwswtsigtdeaidtdcwglsqcdggfscqleptlpdlpspfestvg | |
| kaplppvssdiaraasaqrelfddlsavsqeleeillavtvewpkqeiwthpigmffna | |
| srrlltvlrqqaqadchqgtldeclrtknlftavhcyilnvriltaiselllsqirrtq | |
| nshmsplegsrsqspsrddtssssghssvdtipffsenlpigelfsyvdplthalfsac | |
| ttlhvgvqllreneitlgvhsaqgiaasismsgepgediartgatnsarceeqpttpaa | |
| rvlfmflsdegafqeaksagsrgrtiaalrrcyedifslarkhkhgmlrdlnnipp | |
| lovE-5 | |
| maadqgiftnsvtlspvegsrtggtlprrafrrscdrchaqkikctgnkevtgrapcqr | (SEQ ID NO: 45) |
| cqqaglrcvyserrpkrklrqsraadlvsadpdpclhmssppvpsqslpldvseshssn | |
| tsrqfldppdsydwswtsigtdeaidtdcwglsqcdggfscqleptlpdlpspfestve | |
| kaplppvssdiaraasaqrelfddlsavsqeleeillavtvewpkqeiwthpigmffna | |
| srrlltvlrqqaqadchqgtldeclrtknlftavhcyilnvriltaiselllsqirrtq | |
| nshmsplegsrsqspsrddtssssghssvdtipffsenlpigelfsyvdplthalfsac | |
| ttlhvgvqllreneitlgvhsaqgiaasismsgepgediartgatnsarceeqpttpaa | |
| rvlfmflsdegafqeaksagsrgrtiaalrrcyedifslarkhkhgmlrdlnnipp | |
| lovE-6 | |
| maadqgiftnsvtlspvegsrtggtlprrafrrscdrchaqkikctgnkevtgrapcqr | (SEQ ID NO: 46) |
| cqqaglrcvysercpkrklrqsraadlvsadpdpclhmssppvpsqslpldvseshssn | |
| tsrqfldppdsydwswtsigtdeaidtdcwglsqydggfscqleptlpdlpspfestve | |
| kaplppvssdiaraasaqrklfddlsavsqeleeillavtvewpkqeiwthpigmffna | |
| srrlltvlrqqaqadchqgtldeclrtknlftavhcyilnvrilaaiselllsqirrtq | |
| nshmsplegsrsqspsrddtssssghssvdtipffsenlpigelfsyvdplthalfsac | |
| ttlhvgvqllreneitlgvhsaqgiaasismsgepgediartgatnsarceeqpttpaa | |
| rvlfmflsdegafqeaksagsrgrtiaalrrcyedifslarkhkhgmlrdlnnipp | |
| lovE-7 | |
| maadqgiftnsvtlspvegsrtggtlprrafrrscdrchaqkikctgnkevtgrapcqr | (SEQ ID NO: 47) |
| cqqaglrcvyserrpkrklrqsraadlvsadpdpclhmssppvpsqslpldvseshssn | |
| tsrqfldppdsydwswtsigtdeaidtdcwglsqcdggfscqleptlpdlpsffestve | |
| kaplppvssdiaraasaqrelfddlsavsqeleeillavtvewpkqeiwthpigmffna | |
| srrlltvlrqqaqadchqgaldeclrtknlftavhcyilnvriltaiselllsqirrtq | |
| nshmsplegsrsqspsrddtssssghssvdtipffsenlpigelfsyvdplthalfsac | |
| ttlhvgvqllreneitlgvhsaqgiaasismsgepgediartgatnsarceeqpttpaa | |
| fvlfmflsdegafqeaksagsrgrtiaalrrcyedifslarkhkhgmlrdlnsipp | |
| lovE-8 | |
| maadqgiftnsvtlspvegsrtggtlprrafrrscdrchaqkikctgnkevtgrapcqr | (SEQ ID NO: 48) |
| cqqaglrcvyserrpkrklrqsraadlvsadpdpclhmssppvpsqslpldvseshssn | |
| tsrqfldppdsydwswtsigtdeaidtdcwglsqcdggfscqleptlpdlpspfeetve | |
| kaplppvssdiaraasaqrelfddlsavsqeleeillavtvewpkqeiwthpigmffna | |
| srrlltvlrqqaqadchqgaldeclrtknlftavhcyilnvriltaiselllsqirrtq | |
| nshmsplegsrsqspsrddtssssghssvdtipffsenlpigelfsyvdplthalfsac | |
| ttlhvgvqllreneitlgvhsaqgiaasismsgepgediartgatnsarceeqpttpaa | |
| rvlfmflsdegafqeaksagsrgrtiaalrcyedifslarkhkhgmlrdlnsipp | |
| lovE-9 | |
| maadqgiftnsvtlspvegsrtggtlprrafrrscdrchaqkikctgnkevtgrapcqr | (SEQ ID NO: 49) |
| cqqaglrcvyserrpkrklrqsraadlvsadpdpclhmssppvpsqslpldvseshssn | |
| tsrqfldppdsydwswtsigtdeaidtdcwglsqcdggfscqleptlpdlpspfestve | |
| kaplppvssdiaraasaqrelfddlsavaqeleeillavtvewpkqeiwthpigmffna | |
| srrlltvlrqqaqadchqgaldeclrtknlftavhcyilnvriltaiselllsqirrtq | |
| nshmsplegsrsqspsrddtssssghssvdtipffsenlpigelfsyvdplthalfsac | |
| ttlhvgvqllreneitlgvhsaqgiaasismsgepgediartgatnsarceeqpttpaa | |
| rvlfmflsdegafqeaksagsrgrtiaalrrcyedifslakhkhgmlrdlnsipp | |
| lovE-10 | |
| maadqgiftnsvtlspvegsrtggtlprrafrrscdrchaqkikctgnkevtgrapcqr | (SEQ ID NO: 50) |
| cqqaglrcvyserrpkrklrqsraadlvsadpdpclhmssppvpsqslpldvseshssn | |
| tsrqfldppdsydwswtsigtdeaidtdcwglsqcdggfscqleptlpdlpspfestve | |
| kaplppvssdiaraasaqrelfddlsavsqeleeillavtvewpkqeiwthpigmffna | |
| srrlltvlrqqaqadchqgaldeclrtknlftavhcyilnvriltaiselllsqirrtq | |
| nshmsplegsrsqspsrddtssssghssvdtipffsenlpigelfsyvdplthalfsac | |
| ttlhvgvqllreneitlgvhsaqgiaasismsgepgediartgatnsarceeqpttpaa | |
| rvlfmflsdegafqeaksagsrgrtiaalrrcyedifslarkhkhgmlrdlnsipp | |
| lovE-16 | |
| maadqgifmnsvtlsavegsrtsgtlprrafrrscdrchakkikctgnkevtgrapcqr | (SEQ ID NO: 51) |
| cqqaglrcvysercpkrklrqsraadlvsadpdpclhmssppvpsqslpldvseshssn | |
| tsrqfldppdsydwswtsigtdeaidtdcwglsqcdggfscqleptlpdlpspfestve | |
| kaplppvssdiaraasaqrelfddlsavsqeleeillavtvewpkqeiwthpigmffna | |
| srrlltvlrqqaqadchqgtldeclrtknlftavhcyilnvriltaiselllsqirrtq | |
| nshmsplegsrsqspsrddtssssghssvdtipffsenlpigelfsyvdplthalfsac | |
| ttlhvgvellreneitlgvhsaqgiaasismsgepgediartgatnsarceeqpttpaa | |
| rvlfmflsdegafqeaksagsrgrtiaalrrcyedifslarkhkhgmlrdlnnipp | |
| lovE-19 | |
| maadqgiftnsvtlspvegshtggtlprrafrracdrchaqkikctgnkevtgrapcqr | (SEQ ID NO: 52) |
| cqqaglrcvysercpkrklrhsrasdlvsadpdpclhmssppvpsqslpldvseshssn | |
| tsrqfldppdsydwswtsigtdeaidtdcwglsqcdggfscqleptlpdlpspfestve | |
| kaplppvssdiaraasaqrelfddlsavsqeleeillavtvewpkqeiwthpigmffna | |
| srrlltvlrqqaqadchqgtldeclrtknlftavhcyilnvriltaiselllsqirrtq | |
| nshmspldgsrsqspsrddtssssghssvdtipffsenlpigelfsyvdplthalfsac | |
| ttlhvgvqllreneitlgvdsaqgiaasismsgepgediartgatnsarceeqpttpaa | |
| rvlfmflsdegafqeaksagsrgrtitvlrrsyedifslarkhkhgmlrdlnnips | |
| lovE-20 | |
| maadqgiftnsvtlspvegsrtggtlprralrrscdrchaqkikctgnkevtgrapcqr | (SEQ ID NO: 53) |
| cqqaglrcvysercpkrklrqsraadlvsadpdpclhmssppvpsqslpldvseshssn | |
| tsrqfldppdsydwswtsigtdeaidtdcwglsqcdggfscqleptlpdlpspfestve | |
| kaplppvssdiaraasaqrelfddlsavsqeleeillavtvewpkqeiwthpigmffna | |
| srrlltvlrqqaqadchqgtldeclrtknlftavhcyilnvriltaiselllsqirrtq | |
| nshmsplegsrsqspsrddtssssghssvdtipffsenlpigelfsyvdplthalfsac | |
| ttlhvgvqllreneitlgvhsaqgiaasismsgepgediartgatnsarceeqpitpaa | |
| rvlfmflsgdegafqeaksagsrgrtiaalrrcyedifslarkhkhgmlrdlnnipp | |
| lovE-21 | |
| maadqgiftnsvtlspvegsrtggtlprralrrscdrchaqkikctgnkevtgrapcqr | (SEQ ID NO: 54) |
| cqqaglrcvysercpkrklrqsraadlvsadpdpclhissppvpsqslpldvsdshssn | |
| tsrqfldppdsydwswtsigtdeaidtncwglsqcdggfscqlestlpdlpspfestve | |
| kaplppvssdiaraasaqrelfddlsavsqeleeillavtvewpkqeiwthpigmffna | |
| srrlltvlrqqaqadchqgtldeclrtknlftavhcyilnvriltaiselllsqirrtq | |
| nshmsplegsrsqspsrddtssssghssvdtipffsenlpigelfsyvdplthalfsac | |
| ttlhvgvqllreieitlgvhsaqgiaasismsgepgediartgatnsarceeqpttpaa | |
| rvlfmflsdegafqeaksagsrgrtiaalrrcyedifslarkhkygmlrdlnnipp | |
| lovE-30 | |
| maadqgiftnsvtlspvegsrtggtlprrafrrscdrchaqkvkctgnkevtgrapcqr | (SEQ ID NO: 55) |
| cqqaglrcvysercpkrklrqsraadlvsadpdpclhmssppvpsqslpldvseshssn | |
| tsrqfldppdsydwswtsigtdeaidtdcwglsqcdggfscqleptlpdlpspfestve | |
| kaplppvssdiaraasaqrelfddlsavsqeleeillavtvewpkqeiwthpigmffna | |
| srrlltvlrqqaqadchqgtldeclrtknlftavhcyilnvriltaiselllsqirrtl | |
| nshmsplegsrsqspsrddtssssghssvdtipffsenlpigelfsyvdplthalfsac | |
| ttlhvgvqllreneitlgvhsaqgiaasismsgepgediartgatnsarceeqpttpaa | |
| rvlfmflsdegafqeaksagsrgrtiaalrrcyedifslarkhkhgmlrdlnnippc | |
| lovE-31 | |
| maadqgiftnsvtlspvegsrtggtlprralrrscdrchaqkikctgnkevtgrapcqr | (SEQ ID NO: 56) |
| cqqaglrcvysercpkrklrqsraadlvsadpdpclhmsspsvpsqslpldvseshssn | |
| tsrqfldppdsydwswtsigtdeaidtdcwglsqrdggfssqlkptlpdlpspfestve | |
| kaplppvssdiaraasaqrelfddlsavsqeleeillavtvewpkqeiwthpigmffna | |
| srrlltvlrqqaqadchqgtldeclrtknlftavhcyilnvriltaiselllsqirltq | |
| nshmsplegsrsqspnrddtssssghssvdtipffsenlpigelfsyvdplthalfsac | |
| ttlhvgvqllreneitlgvhsaqgiaasismsgepgediartgatnsarceeqpttpaa | |
| rvlfmflsdegafqeaksagsrgrtiaalrrcyedifslarkhkhgmlrdlnnipp | |
| lovE-32 | |
| maadqgiftnsvtispvvgsrtggtlprrafrrscdrchaqkikctgnkevtgrapcqr | (SEQ ID NO: 57) |
| cqqaglrcvysercpkrklrqsraadlvsadpdpclhmssppvpsqslpldvseshssn | |
| tsrqfldppdsydwswtsictdeaidtdcwglsqcdggfscqleptlpdlpspfestve | |
| kaplppvssdiaraasaqrelfddlsavsqeleeillavtvewpkqeiwthpigmffna | |
| srrlltvlrqqaqadchqgtldeclrtknlftavhcyilnvriltaiselllsqirrtq | |
| nshmsplegsrsqspsrddtssssghssvdtipffsenlpigglfsyvdplthalfsac | |
| ttlhvglqllreneitlgvhsaqgiaasismsgesgediartgatssarceeqpttpaa | |
| rvlfmflsdegafqeaksagsrgrtiaalrrcyedifslarkhkhgmlrdlnnipp | |
| lovE-33 | |
| maadqgiftnsvtlspvegsrtggtlprrafrrscdrcharkikctgnkevtgrapcqr | (SEQ ID NO: 58) |
| cqqaglrcvysercpkrklrqsraadlvsadpdpclhmssppvpsqslpldvseshssn | |
| tsrqfldppdsydwswtsigtdeaidtdcwglsqcdggfscqleptlpdlpspfeytve | |
| kaplppvssdiaraasaqrelfddlsavsqeleeillavtvewpkqeiwthpigmffna | |
| srrlltvlrqqaqadchqgtldeclrtknlftavhcyilnvriltaiselllsqirrtq | |
| nshmsplegsrsqspsrddtssssghssvdtipffsenlpigelfsyvdplthalfsac | |
| ttlhvgvqllreneitlgvhsaqgiaasismsgepgediartgatnstrceeqpttpaa | |
| rvlfmflsdegafqeaksagsrgrtiaalrrcyedifslarkhkhgmlrdlnnipp | |
| lovE-34 | |
| maadqgiftnsvtlspvegsrtggtlprralrrscdrchaqkikctgnkevigrapcqr | (SEQ ID NO: 59) |
| cqqaglrcvysercpkrklrqsraadlvsadpdpclhmsspqvpsqslsldiseshssn | |
| tsrqfldppdsydwswtsigtdeaidtdcwglsqcdggfscqleptlpdlpspfestve | |
| kaplppvssdiaraasaqrelfddlsavsqeleeillavtvewpkqeiwthpigmffna | |
| srrlltvlrqqaqadchqgtldeclrtknlftavhcyilnvriltaiselllsqirrtq | |
| nshmsplegsrsqspsrddtssssghssvdtipffsenlpigelfsyvdplthalfsac | |
| ttlhvgvqllreneitlgvhsaqgiaasismsgepgediartgatnsarceeqpttpaa | |
| rvlfmflsdegafqeaksagsrgrtiaalrrcyedifslarkhkhgmlrdlnnipp | |
| lovE-36 | |
| maadqgiftnsvtlspvegsrtggtlprrafrrscdrchaqkikctgnkevtgrapcqr | (SEQ ID NO: 60) |
| cqqaglrcvysercpkrklrqsraanlvsadpdpclhmssppvpsqslpldvseshssn | |
| tsrqfldppdsydwswtsigtdeafdtdcwglsqcdggfscqleptlpdlpspfestve | |
| kaplppvssdiaraasaqrelfddlsavsqeleeillavtvewpkqeiwthpigiffna | |
| srrlltvlrqqaqadchqgtldeclrtknlftavhcyilnvriltaiselllsqirrtq | |
| nshmsplegsrsqspsrddissssghssvdtipffsenlpigelfsyvdplthalfsac | |
| ttlhvgvqllreneitlgvhsaqgiaayisksgepgediartgatnsarceeqpttpaa | |
| rvlfmflsdegafqeaksagsrgrtiaalrrcyedifslarkhkhgmlrdlnnipp | |
| lovE-37 | |
| maadqgiftnsvtlspvegsrtggtlprrafrrscdrchaqkikcignkevtgrapcqr | (SEQ ID NO: 61) |
| cqraglrcvysercpkrrlrqsraadlvsadpdpclhmssppvpsqslpldvseshssn | |
| tsrqfldppdsydwswtsigtdeaidtdcwglsqcdggfscqleptlpdlpspfestve | |
| kaplppvssdiaraasaqrelfddlsavsqeleeillavtvewpkqeiwthpigmffna | |
| srrlltvlrqqaqadchqgtldeclrtknlftavhcyilnvriltaiselllsqirrtq | |
| nshmsplegsrsqspsrddtssssghscvdtipffsenlpigelfsyvdplthalfsac | |
| ttlhvgvqllreyeitlgihsaqgiaasismsgepgediartgatnsarceeqpttpaa | |
| rvlfmflsdegafqeaksagsrgrtiaalrrcyedifslarkhkhgmlrdlnnipp | |
| lovE-38 | |
| maadqgiftnsvtlspvegsrtggtlprrafrrscdrcharkikctgnkevtgrapcqr | (SEQ ID NO: 62) |
| cqqaglrcvysercpkrklrqsraadlvsadpdpclhmssppvpsqslpldvseshssn | |
| tsrqfldppdsydwswtsigtdeaidtdcwglsqcdggfscqleptlpdlpspfestve | |
| kaplppvssdiaraasaqrelfddlsavsqeleeillavtvewpkqeiwthpigmffna | |
| srrlltvlrqqaqadchqgtldeclrtknlftavhcyilnvriltaiselllsqirriq | |
| nshmsplegsrsqslsrddtssssghssvdtipffsenlpidelfsyvdplthalfsac | |
| ttlhvgvqllreneitlgvhsaqgiaasismsgelgedivrtgatnsarceeqpttpaa | |
| rvlfmflsdegafqeaksagsrsrtiaalrrcyedifslarkhkhgmlrdlnnipp | |
| lovE-39 | |
| maadqgiftnsvtlspvegsrtggtlprrafrrscdrchaqkikctgnkevngrapcqr | (SEQ ID NO: 63) |
| cqqaglrcvysercpkrklrqsraadlvsadpdpclhmssppvpsqslpldiseshssn | |
| tsrqfldppdsydwswtsigideaidtdcwglsqcdggfscqleptlpdlpspfestve | |
| kaplppissdiaraasaqrelfddlsavsqeleeillavtvewpkqeiwthpigmffna | |
| srrlltvlrqqaqadchqgtldeclrtknlftavhcyilnvrilaaiselllsqirrtq | |
| nshmsplegsrsqspsrddtssssghssvdtipffsenlpigelfsyvdplthalfsac | |
| ttlhvgvqllreneitlgvhsaqgiaasismsgepgediartgatnsarceeqpttpaa | |
| rvlfmflsdegafqeaksagsrgrtiaalrrcyedifslarkhkhgmlrdlnnipp | |
| lovE-40 | |
| maaeqgiftnsvtlspvegsrtggtlprrafrrscdrcharkikctgnkevtgrapcqr | (SEQ ID NO: 64) |
| cqqaglrcvysercpkrklrqsraadlisadpdpclhmssppvpsqslplevseshssn | |
| tsrqfldppdsydwswtsigtdkaidtdcwglsgcdggfscqleptlpdlpspfestve | |
| kaplppvssditraasaqrelfddlsavsqeleeillavtvewpkqeiwthpigmffna | |
| srrlltvlrqqaqadchqgtldeclrtknlftavhcyildvriltaiselllsqirrtq | |
| nshmsplegsrsqspsrddtssssghssvdtipffsenlpigelfsyvdplrhalfsac | |
| ttlhvgvqllreieitlgvhsargiaasismsgepgediartgatnsarceeqpttpaa | |
| rvlfmflsdegtfqeaksagsrgrtiaalrrcyedifslarkhkhgmlrdlnnipp | |
| lovE-41 | |
| maadqgiftnsvtlspvegsrtggtlprrafrrscdrchaqkikctgnkevtgrapcqr | (SEQ ID NO: 65) |
| cqqaglrcvysercpkrklrqsraadlvsadpdpclhmssppvpsqslpldvseshssn | |
| tsrqfldppdsynwlwtsigtdeaidtdcwglsqcdggfscqleptlpdlpspfestve | |
| kaplppvssdiaraasaqrelfddlsavsqeleeillavtvewpkqeiwthpigmffna | |
| srrlltvlrqqaqadchqgtldeclrtknlftavhcyilnvriltaiselllsqirrtq | |
| nshmsplegsrsqspsgddtssssghssvdtipffsenlpigelfsyvdplthalfsac | |
| ttlhvgvqllreneitlgvhsaqgiaasismsgepgediartgatnsarceeqpttpaa | |
| rvlfmflsdegafqegksagsrgrtiaalrrcyedifslarkhkhgmlrdlnnipp | |

TABLE 6
DNA Sequences of Variants of the lovE Gene

| | |
|---|---|
| lovE-1 | |
| ATGGCTGCAGATCAAGGTATATTCACGAACTCGGTCACTCTCTCGCCAGTGGAGGGTTC | (SEQ ID NO: 66) |
| ACGCACCGGTGGAACATTACCCCGCCGTGCATTCCGACGCTCTTGTGATCGGTGTCATG | |
| CACAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGATGCCCCAAGCGCAAGCTACG | |
| CCAATCCAGGGCAGCGGATCTCGTCTCTGCTGACCCAGATCCCTGCTTGCACATGTCCT | |
| CGCCTCCAGTGCCCTCACAGAGCTTGCCGCTAGACGTATCCGAGTCGCATTCCTCAAAT | |
| ACCTCCCGGCAGTTTCTTGATCCACCGGACAGCTACGACTGGTCGTGGACCTCGATTGG | |
| CACTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCGTCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGAGCTGTTCCCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-2 | |
| ATGGCTGCAGATCAAGGTATATTCACGAACTCGGTCACTCTCTCGCCAGTGGAGGGTTC | (SEQ ID NO: 67) |
| ACGCACCGGTGGAACATTACCCCGCCGTGCATTCCGACGCTCTTGTGATCGGTGTCATG | |
| CACAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGATGCCCCAAGCGCAAGCTACG | |
| CCAATCCAGGGCAGCGGATCTCGTCTCTGCTGACCCAGATCCCTGCTTGCACATGTCCT | |
| CGCCTCCAGTGCCCTCACAGAGCTTGCCGCTAGACGTATCCGAGTCGCATTCCTCAAAT | |
| ACCTCCTGGCAATTTCTTGATCCACCGGACAGCTACGACTGGTTGTGGACCTCGATTGG | |
| CACTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAAGCAGA | |
| GCTTTTCGATGACCTGTCGGCGGTGTCGCAGGAACTGGAAGAGATCCTTCTGGCCGTGA | |
| CGGTAGAGTGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAC | |
| CAGCAGCAGCACCGGCCACGGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-3 | |
| ATGGCTGCAGATCAAGGTATATTCACGAACTCGGTCACTCTCTCGCCAGTGGAGGGTTC | (SEQ ID NO: 68) |
| ACGCACCGGTGGAACATTACCCCGCCGTGCATTCCGACGCTCTTGTGATCGGTGTCATG | |
| CACAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGACGCCCCAAGCGCAAGCTACG | |
| CCAATCCAGGGTAGCGGATCTCGTCTCTGCTGACCCAGATCCCTGCTTGCACATGTCCT | |
| CGCCTCCAGTGCCCTCACAGAGCTTGCCGCTAGACGTATCCGAGTCGCATTCCTCAAAT | |
| ACCTCCCGGCAATTTCTTGATCCACCGGACAGCTACGACTGGTCGTGGATCTCGATTGG | |
| CACTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-4 | |
| ATGGCTGCAGATCAAGGTATATTCACGAACTCGGTCACTCTCTCGCCAGTGGAGGGTTC | (SEQ ID NO: 69) |
| ACGCACCGGTGGAACATTACCCCGCCGTGCATTCCGACGCTCTTGTGATCGGTGTCATG | |
| CACAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGACGCCCCAAGCGCAAGCTACG | |
| CCAATCCAGGGCAGCGGATCTCGTCTCTGCTGACCCAGATCCCTGCTTGCACATGTCCT | |
| CGCCTCCAGTGCCCTCACAGAGCTTGCCGCTAGACGTATCCGAGTCGCATTCCTCAAAT | |
| ACCTCCCGGCAATTTCTTGATCCACCGGACAGCTACGACTGGTCGTGGACCTCGATTGG | |
| CACTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTCTACGGTTGGA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-5 | |
| ATGGCTGCAGATCAAGGTATATTCACGAACTCGGTCACTCTCTCGCCAGTGGAGGGTTC | (SEQ ID NO: 70) |
| ACGCACCGGTGGAACATTACCCCGCCGTGCATTCCGACGCTCTTGTGATCGGTGTCATG | |
| CACAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGACGCCCCAAGCGCAAGCTACG | |
| CCAATCCAGGGCAGCGGATCTCGTCTCTGCTGACCCAGATCCCTGCTTGCACATGTCCT | |
| CGCCTCCAGTGCCCTCACAGAGCTTGCCGCTAGACGTATCCGAGTCGCATTCCTCAAAT | |
| ACCTCCCGGCAATTTCTTGATCCACCGGACAGCTACGACTGGTCGTGGACCTCGATTGG | |
| CACTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGACTACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-6 | |
| ATGGCTGCAGATCAAGGTATATTCACGAACTCGGTCACTCTCTCGCCAGTGGAGGGTTC | (SEQ ID NO: 71) |
| ACGCACCGGTGGAACATTACCCCGCCGTGCATTCCGACGCTCTTGTGATCGGTGTCATG | |
| CACAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGATGCCCCAAGCGCAAGCTACG | |
| CCAATCCAGGGCAGCGGATCTCGTCTCTGCTGACCCAGATCCCTGCTTGCACATGTCCT | |
| CGCCTCCAGTGCCCTCACAGAGCTTGCCGCTAGACGTATCCGAGTCGCATTCCTCAAAT | |
| ACCTCCCGGCAATTTCTTGATCCACCGGACAGCTACGACTGGTCGTGGACCTCGATTGG | |
| CACTGACGAGGCTATTGACACTGACTGCTGGGGGCTGTCCCAATATGATGGAGGCTTCA | |
| GCTGTCAGTTAGAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAAA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGGCCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGACAGGTCAACAATATTCCTCCATGA | |
| lovE-7 | |
| ATGGCTGCAGATCAAGGTATATTCACGAACTCGGTCACTCTCTCGCCAGTGGAGGGTTC | (SEQ ID NO: 72) |
| ACGCACCGGTGGAACATTACCCCGCCGTGCATTCCGACGCTCTTGTGATCGGTGTGATG | |
| CACAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGACGCCCCAAGCGCAAGCTACG | |
| CCAATCCAGGGCAGCGGATCTCGTCTCTGCTGACCCAGATCCCTGCTTGCACATGTCCT | |
| CGCCTCCAGTGCCCTCACAGAGCTTGCCGCTAGACGTATCCGAGTCGCATTCCTCAAAT | |
| ACCTCCCGGCAATTTCTTGATCCACCGGACAGCTACGACTGGTCGTGGACCTCGATTGG | |
| CACTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCGC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAGTATTCCTCCATGA | |
| lovE-8 | |
| ATGGCTGCAGATCAAGGTATATTCACGAACTCGGTCACTCTCTCGCCAGTGGAGGGTTC | (SEQ ID NO: 73) |
| ACGCACCGGTGGAACATTACCCCGCCGTGCATTCCGACGCTCTTGTGATCGGTGTCATG | |
| CACAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGACGCCCCAAGCGCAAGCTACG | |
| CCAATCCAGGGCAGCGGATCTCGTCTCTGCTGACCCAGATCCCTGCTTGCACATGTCCT | |
| CGCCTCCAGTGCCCTCACAGAGCTTGCCGCTAGACGTATCCGAGTCGCATTCCTCAAAT | |
| ACCTCCCGGCAATTTCTTGATCCACCGGACAGCTACGACTGGTCGTGGACCTCGATTGG | |
| CACTGACGTGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-9 | |
| ATGGCTGCAGATCAAGGTATATTCACGAACTCGGTCACTCTCTCGCCAGTGGAGGGTTC | (SEQ ID NO: 74) |
| ACGCACCGGTGGAACATTACCCCGCCGTGCATTCCGACGCTCTTGTGATCGGTGTCATG | |
| CACAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGACGCCCCAAGCGCAAGCTACG | |
| CCAATCCAGGGCAGCGGATCTCGTTTCTGCTGACCCAGATCCCTGCTTGCACATGTCCT | |
| CGCCTCCAGTGCCCTCACAGAGCTTGCCACTAGACGTATCCGAGTCGCATTCCTCAAAT | |
| ACCTCCCGGCAATTTCTTGATCCACCGGACAGCTACGACTGGTCGTGGACCTCGATTGG | |
| CACTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ACGTGCGGATTTTGACCGCCATATCGGAGTTGCTCCTGTCGCAAATTAGGCGGACCCAG | |
| AACAGCCATATGAGCCCACTGAAAGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-10 | |
| ATGGCTGCAGATCAAGGTATATTCACGAACTCGGTCACTCTCTCGCCAGTGGAGGGTTC | (SEQ ID NO: 75) |
| ACGCACCGGTGGAACATTACCCCGCCGTGCATTCCGACGCTCTTGTGATCGGTGTCATG | |
| CAAAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGATGCCCCAAGCGCAAGCTACG | |
| CCAATCCAGGGCAGCGGATCTCGTCTCTGCTGACCCAGATCCCTGCTTGCACATGTCCT | |
| CGCCTCCAGTGCCCTCACAGAGCTTGCCGCTAGACGTATCCGAGTCGCATTCCTCAAAT | |
| ACCTCCCGGCAATTTCTTGATCCACCGGACAGCTACGACTGGTCGTGGACCTCGATTGG | |
| CACTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGCTACATGTTGGGGTACAATTGCTGCGTGAGAATGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-16 | |
| ATGGCTGCAGATCAAGGTATATTCATGAACTCGGTCACTCTCTCTGCAGTGGAGGGTTC | (SEQ ID NO: 76) |
| ACGCACCAGTGGAACATTACCCCGCCGTGCATTCCGACGCTCTTGTGATCGGTGTCATG | |
| CAAAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGATGCCCCAAGCGCAAGCTACG | |
| CCAATCCAGGGCAGCGGATCTCGTCTCTGCTGACCCAGATCCCTGCTTGCACATGTCCT | |
| CGCCTCCAGTGCCCTCACAGAGCTTGCCGCTAGACGTATCCGAGTCGCATTCCTCAAAT | |
| ACCTCCCGGCAATTTCTTGATCCACCGGACAGCTACGACTGGTCGTGGACCTCGATTGG | |
| CACTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTCTACAGTTGAA | |
| AAAGCTGGCTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTATCGCAAATTAGGCGGACCCAG | |
| AACAGCCATATGAGCCCACTGGAAGGGAGTCGATCCCAGTCGCCGAGCAGAGACGACAC | |
| TAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTAGAATTGCTGCGTGAGAATGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-19 | |
| ATGGCTGCAGATCAAGGTATATTCACGAACTCGGTCACTCTCTCGCCAGTGGAGGGTTC | (SEQ ID NO: 77) |
| ACACACCGGTGGAACATTACCCCGCCGTGCATTCCGACGCGCTTGTGATCGGTGTCATG | |
| CACAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGATGCCCCAAGCGCAAGCTACG | |
| CCATTCCAGGGCATCGGATCTCGTCTCTGCTGACCCAGATCCCTGCTTGCACATGTCCT | |
| CGCCTCCAGTGCCCTCACAGAGCTTGCCGCTAGACGTATCCGAGTCGCATTCCTCAAAT | |
| ACCTCCCGGCAATTTCTTGATCCACCGGACAGCTACGACTGGTCGTGGACCTCGATTGG | |
| CACTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCAG | |
| AACAGCCATATGAGCCCACTGGACGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGAGCTATTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTAGA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCACAGTACTGCGACGAAGCTATGAGGATATCTTTTCCCTCG | |
| CCCGCAAACACAAACATGGCATGCTCAGAGACCTCAACAATATTCCTTCATGA | |
| lovE-20 | |
| ATGGCTGCAGATCAAGGTATATTCACGAACTCGGTCACTCTCTCGCCAGTGGAGGGTTC | (SEQ ID NO: 78) |
| ACGCACCGGTGGAACATTACCCCGCCGTGCACTCCGACGCTCTTGTGATCGGTGTCATG | |
| CACAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGATGCCCCAAGCGCAAGCTACG | |
| CCAATCCAGGGCAGCGGATCTCGTCTCTGCTGACCCAGATCCCTGCTTGCACATGTCCT | |
| CGCCTCCAGTGCCCTCACAGAGCTTGCCGCTAGACGTATCCGAGTCGCATTCCTCAAAT | |
| ACCTCCCGGCAATTTCTTGATCCACCGGACAGCTACGACTGGTCGTGGACCTCGATTGG | |
| CACTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGATCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-21 | |
| ATGGCTGCAGATCAAGGTATATTCACGAACTCGGTCACTCTCTCGCCAGTGGAGGGTTC | (SEQ ID NO: 79) |
| ACGCACCGGTGGAACATTACCCCGCCGTGCACTCCGACGCTCTTGTGATCGGTGTCATG | |
| CACAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGATGCCCCAAGCGCAAGCTACG | |
| CCAATCCAGGGCAGCGGATCTCGTCTCTGCTGACCCAGATCCCTGCTTGCACATATCCT | |
| CGCCTCCAGTGCCCTCACAGAGCTTACCGCTAGACGTATCCGATTCGCATTCCTCAAAT | |
| ACCTCCCGGCAATTTCTTGATCCACCGGACAGCTACGACTGGTCGTGGACCTCGATTGG | |
| CACTGACGAGGCTATTGACACTAACTGCTGGGGGCTGTCCCAATGTGATGGAGGCTTCA | |
| GCTGTCAGTTAGAGTCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCTAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGATTGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCAACCAATTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAATATGGCATGCTCAGAGACCTCAACAATATTCCTCCATGA | |
| lovE-30 | |
| ATGGCTGCAGATCAAGGTATATTCACGAACTCGGTCACTCTCTCGCCAGTGGAGGGTTC | (SEQ ID NO: 80) |
| ACGCACCGGTGGAACATTACCCCGCCGTGCATTCCGACGCTCTTGTGATCGGTGTCATG | |
| CACAAAAGGTCAAATGTACTGGAAATAAGGAGGTTACTGGCCGTGCTCCCTGTCAGCGT | |
| TGCCAGCAGGCTGGACTTCGATGCGTCTACAGTGAGCGATGCCCCAAGCGCAAGCTACG | |
| CCAATCCAGGGCAGCGGATCTCGTCTCTGCTGACCCAGATCCCTGCTTGCACATGTCCT | |
| CGCCTCCAGTGCCCTCACAGAGCTTGCCGCTAGACGTATCCGAGTCGCATTCCTCAAAT | |
| ACCTCCCGGCAATTTCTTGATCCACCGGACAGCTACGACTGGTCGTGGACCTCGATTGG | |
| CACTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCTG | |
| AACAGCCATATGAGCCCACTGGAAGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-31 | |
| ATGGCTGCAGATCAAGGTATATTCACGAACTCCGTCACTCTCTCGCCAGTGGAGGGTTC | (SEQ ID NO: 81) |
| ACGCACCGGTGGAACATTACCCCGCCGTGCATTACGACGCTCTTGTGATCGGTGTCATG | |
| CACAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGATGCCCCAAGCGCAAGTTACG | |
| CCAATCCAGGGCAGCGGATCTCGTCTCTGCTGACCCAGATCCCTGCTTGCACATGTCCT | |
| CGCCTTCAGTGCCCTCACAGAGCTTGCCGCTAGACGTATCCGAGTCGCATTCCTCAAAT | |
| ACCTCCCGGCAATTTCTTGATCCACCGGACAGCTACGACTGGTCGTGGACCTCGATTGG | |
| CACTGACGAGGCTATTGACACTGACTGCTGGGGGCTGTCCCAACGTGATGGAGGCTTCA | |
| GCTCTCAGTTAAAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTACTGTCGCAAATTAGGCTGACCCAG | |
| AACAGCCATATGAGCCCACTGGAAGGGAGTCGATCCCAGTCGCCGAACAGAGACGACAC | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-32 | |
| ATGGCTGCAGATCAAGGTATATTCACTAACTCGGTCACTATCTCGCCAGTGGTGGGTTC | (SEQ ID NO: 82) |
| ACGCACCGGTGGAACATTACCCCGCCGTGCATTCCGACGCTCTTGTGATCGGTGTCATG | |
| CACAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGATGCCCCAAGCGCAAGCTACG | |
| CCAATCCAGGGCAGCGGATCTCGTCTCTGCTGACCCAGATCCCTGCTTGCACATGTCCT | |
| CGCCTCCAGTGCCCTCACAGAGTTTGCCGCTAGACGTATCCGAGTCGCATTCCTCAAAT | |
| ACCTCCCGGCAATTTCTTGATCCACCGGACAGCTACGACTGGTCGTGGACCTCGATTTG | |
| CACTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGGGCTGTTCTCCTATGTTGACCCCCTGACACACGCCCTATTCTCGGCTTGC | |
| ACTACGTTACATGTTGGGCTACAATTGCTGCGTGAGAATGAGATTACTCTGGGAGTACA | |
| CTCCGCCCAGGGCATTGCAGCTTCCATCAGCATGAGCGGGGAATCAGGCGAGGATATAG | |
| CCAGGACAGGGGCGACCAGTTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-33 | |
| ATGGCTGCAGATCAAGGTATATTCACGAACTCGGTCACTCTCTCGCCAGTGGAGGGTTC | (SEQ ID NO: 83) |
| ACGCACCGGTGGAACATTACCCCGCCGTGCATTCCGACGCTCTTGTGATCGGTGTCATG | |
| CACGAAAGATCAAATGTACTGGAAATAAGGAGGTTACTGGCCGTGCTCCCTGTCAGCGT | |
| TGCCAGCAGGCTGGACTTCGATGCGTCTACAGTGAGCGATGCCCCAAGCGCAAGCTACG | |
| CCAATCCAGGGCAGCGGATCTCGTCTCTGCTGACCCAGATCCCTGCTTGCACATGTCCT | |
| CGCCTCCAGTGCCCTCACAGAGCTTGCCGCTAGACGTATCCGAGTCGCATTCCTCAAAT | |
| ACCTCCCGGCAATTTCTTGATCCACCGGACAGCTACGACTGGTCGTGGACCTCGATTGG | |
| CACTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTATACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCGACCAATTCCACAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-34 | |
| ATGGCTGCAGATCAAGGTATATTCACGAACTCGGTCACTCTCTCGCCAGTGGAGGGTTC | (SEQ ID NO: 84) |
| ACGCACCGGTGGAACATTACCCCGCCGTGCATTGCGACGCTCTTGTGATCGGTGTCATG | |
| CACAAAAGATCAAATGTACTGGAAATAAGGAGGTTATTGGCCGTGCTCCCTGTCAGCGT | |
| TGCCAGCAGGCTGGACTTCGATGCGTATACAGTGAGCGATGCCCCAAGCGCAAGCTACG | |
| CCAATCCAGGGCAGCGGATCTCGTCTCTGCTGACCCAGATCCCTGCTTGCACATGTCCT | |
| CGCCTCAAGTGCCCTCACAGAGCTTGTCGCTAGACATATCCGAGTCGCATTCCTCAAAT | |
| ACCTCCCGGCAATTTCTTGATCCACCGGACAGCTACGACTGGTCGTGGACCTCGATTGG | |
| CACTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGGCATTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-36 | |
| ATGGCTGCAGATCAAGGTATATTCACGAACTCGGTCACTCTCTCACCAGTGGAGGGTTC | (SEQ ID NO: 85) |
| ACGCACCGGTGGAACATTACCCCGCCGTGCATTCCGACGCTCTTGTGATCGGTGTCATG | |
| CACAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGATGCCCCAAGCGCAAGCTACG | |
| CCAATCCAGGGCAGCGAATCTCGTCTCTGCTGACCCAGATCCCTGCTTACACATGTCCT | |
| CGCCTCCAGTGCCCTCACAGAGCTTGCCGCTAGACGTATCCGAGTCGCATTCCTCAAAT | |
| ACCTCCCGGCAATTTCTTGATCCACCGGACAGCTACGACTGGTCGTGGACCTCGATTGG | |
| CACTGACGAGGCTTTTGACACTGACTGCTGGGGGCTATCCCAATGTGATGGAGGCTTCA | |
| GCTGTCAGCTAGAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATCTTTTTCAATGCG | |
| TCACGACGGCTTCTTACTGTCCTGCGCCAGCAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAT | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGCATTGCAGCTTACATCAGCAAGAGCGGGGAACCAGGCGAGGATATAG | |
| CCAGGACAGGGGCGACCAATTCCGCAAGATGCGAGGAGCAGCCGACCACTCCAGCGGCT | |
| CGGGTGTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-37 | |
| ATGGCTGCAGATCAAGGTATATTCACGAACTCGGTCACTCTCTCGCCAGTGGAGGGTTC | (SEQ ID NO: 86) |
| ACGCACCGGTGGAACATTACCCCGCCGTGCATTCCGACGCTCTTGTGATCGGTGTCATG | |
| CACAAAAGATCAAATGTATTGGAAATAAGGAGGTTACTGGCCGTGCTCCCTGTCAGCGT | |
| TGCCAACGGGCTGGACTTCGATGCGTCTACAGTGAGCGATGCCCCAAGCGCAGGCTACG | |
| CCAATCCAGGGCAGCGGATCTCGTCTCTGCTGACCCAGATCCCTGCTTGCACATGTCCT | |
| CGCCTCCAGTGCCCTCACAGAGCTTGCCGCTAGACGTATCCGAGTCGCATTCCTCAAAT | |
| ACCTCCCGGCAATTTCTTGATCCACCGGACAGCTACGACTGGTCGTGGACCTCGATTGG | |
| CACTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCT | |
| TCACGACGGCTTCTTACTGTCCTGCGCCAACAAGCTCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAC | |
| CAGCAGCAGCAGCGGCCACAGCTGTGTCGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGTATGAGATTACTCTGGGAATACA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGATCTCAACAATATTCCTCCATGA | |
| lovE-38 | |
| ATGGCTGCAGATCAAGGTATATTCACGAACTCGGTCACTCTCTCGCCAGTGGAGGGTTC | (SEQ ID NO: 87) |
| ACGCACCGGTGGAACATTACCCCGCCGTGCATTCCGACGCTCTTGTGATCGGTGTCATG | |
| CACGAAAGATCAAATGTACTGGAAATAAGGAGGTTACTGGCCGTGCTCCCTGTCAGCGT | |
| TGCCAGCAAGCTGGACTTCGATGCGTCTATAGTGAGCGATGCCCCAAGCGCAAGCTACG | |
| CCAATCCAGGGCAGCGGATCTCGTCTCTGCTGACCCAGATCCCTGCTTGCACATGTCCT | |
| CGCCTCCAGTGCCCTCACAGAGCTTGCCGCTAGACGTATCCGAGTCGCATTCCTCAAAT | |
| ACCTCCCGGCAATTTCTTGATCCACCGGACAGCTACGACTGGTCGTGGACCTCGATTGG | |
| CACTGACGAGGCTATTGACACTGACTGCTGGGGGCTGTCCCAATGTGATGGAGGCTTCA | |
| GCTGTCAGCTAGAGCCAACGCTGCCGGATCTACCTTCGCCCTTCGAGTCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGATCCAG | |
| AACAGCCATATGAGCCCACTGGAAGGGAGTCGATCCCAGTCGCTGAGCAGAGACGACAC | |
| CAGCAGCAGTAGCGGCCACAGCAGTGTTGACACCATACCCTTCTTTAGCGAGAACCTCC | |
| CTATTGATGAGCTGTTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGCATTGCAGCTTCCATCAGCATGAGCGGGGAACTAGGCGAGGATATAG | |
| TCAGGACAGGGGCGACCAATTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAAGTCGAACCATCGCAGCACTGCGACGATGCTATGAGGATATCTTTTCCCTCG | |
| CCCGCAACACAACATGGCATGCTCAGAGACCTCAACAATATTCCTCCATGA | |
| lovE-39 | |
| ATGGCTGCAGATCAAGGTATATTCACGAACTCGGTCACTCTCTCACCAGTGGAGGGTTC | (SEQ ID NO: 88) |
| ACGCACCGGTGGAACATTACCCCGCCGTGCATTCCGACGCTCTTGTGATCGGTGTCATG | |
| CACAAAAGATCAAATGTACTGGAAATAAGGAGGTTAATGGCCGTGCTCCCTGTCAGCGT | |
| TGCCAGCAGGCTGGACTTCGATGCGTCTACAGTGAGCGATGCCCCAAGCGCAAGCTACG | |
| CCAATCCAGGGCAGCGGATCTCGTCTCTGCTGACCCAGATCCCTGCTTGCACATGTCCT | |
| CGCCTCCAGTGCCCTCCCAGAGCTTGCCGCTAGACATATCCGAGTCGCATTCCTCAAAT | |
| ACCTCCCGGCAATTTCTTGATCCACCGGACAGCTACGACTGGTCGTGGACCTCGATTGG | |
| CATTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAGTCTACGGTTGAA | |
| AAAGCTCCGTTGCCACCGATATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGGCCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGCATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGGCTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-40 | |
| ATGGCTGCAGAACAAGGTATATTCACGAACTCGGTCACTCTCTCGCCAGTGGAGGGTTC | (SEQ ID NO: 89) |
| ACGCACCGGTGGAACATTACCCCGCCGTGCATTCCGACGCTCTTGTGATCGGTGTCATG | |
| CACGAAAGATCAAATGTACTGGAAATAAGGAGGTTACTGGCCGTGCTCCCTGTCAGCGT | |
| TGCCAGCAGGCTGGACTTCGATGTGTCTACAGTGAGCGATGCCCCAAGCGCAAGCTACG | |
| CCAATCCAGGGCAGCGGATCTCATCTCTGCTGACCCAGATCCCTGCTTGCACATGTCCT | |
| CGCCTCCAGTGCCCTCACAGAGCTTGCCGCTAGAAGTATCCGAGTCGCATTCCTCAAAT | |
| ACCTCCCGGCAATTTCTTGATCCACCGGACAGCTACGACTGGTCGTGGACCTCGATTGG | |
| CACTGACAAGGCTATTGACACTGACTGCTGGGGGCTGTCCCAATGTGATGGAGGCTTCA | |
| GCTGTCAGTTAGAGCCAACGCTGCCGGATCTACCTTCGCCCTTTGAGTCTACGGTTGAA | |
| AAAGCTCCGTTGCCACCGGTATCGAGCGACATTACTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGG | |
| ATGTGCGGATTTTGACCGCCATATCGGAGTTGCTCCTGTCGCAAATTAGGCGGACCCAG | |
| AACAGCCATATGAGCCCACTGGAAGGGAGTCGATCCCAGTCGCCGAGCAGAGACGACAC | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGAGACACGCCCTATTCTCGGCTTGC | |
| ACTACGTTACATGTTGGGGTACAATTGCTGCGTGAGATTGAGATTACTCTGGGAGTACA | |
| CTCCGCCCGGGGCATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGACTTTCCAGGAGGCAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |
| lovE-41 | |
| ATGGCTGCAGATCAAGGTATATTCACGAACTCGGTCACTCTCTCGCCAGTGGAGGGTTC | (SEQ ID NO: 90) |
| ACGCACCGGTGGAACATTACCCCGCCGTGCATTCCGACGCTCTTGTGATCGGTGTCATG | |
| CACAAAAGATCAAATGTACTGGAAATAAGGAGGTTACTGGCCGTGCTCCCTGTCAGCGT | |
| TGCCAGCAGGCTGGACTTCGATGCGTCTACAGTGAGCGATGCCCCAAGCGCAAGCTACG | |
| CCAATCCAGGGCAGCGGATCTCGTCTCTGCTGACCCAGATCCCTGCTTGCACATGTCCT | |
| CACCTCCAGTGCCCTCACAGAGCTTGCCGCTAGACGTATCCGAGTCGCATTCCTCAAAT | |
| ACCTCCCGGCAATTTCTTGATCCACCGGACAGCTACAACTGGTTGTGGACCTCGATTGG | |
| CACTGACGAGGCTATTGACACTGACTGCTGGGGGCTGTCCCAATGTGATGGAGGCTTCA | |
| GCTGTCAGTTAGAGCCAACGCTGCCGGATCTACCTTCGCCCTTCGAATCTACGGTTGAA | |
| AAAGCTCCGTTGCCACCGGTATCGAGCGACATTGCTCGTGCGGCCAGTGCGCAACGAGA | |
| GCTTTTCGATGACCTGTCGGCGGTGTCGCAGGAACTGGAAGAGATCCTTCTGGCCGTGA | |
| CGGTAGAATGGCCGAAGCAGGAAATCTGGACCCATCCCATCGGAATGTTTTTCAATGCG | |
| TCACGACGGCTTCTTACTGTCCTGCGCCAACAAGCGCAGGCCGACTGCCATCAAGGCAC | |
| ACTAGACGAATGTTTACGGACCAAGAACCTCTTTACGGCAGTACACTGTTACATATTGA | |
| ATGTGCGGATTTTGACCGCCATATCGGAGTTGCTCCTGTCGCAAATTAGGCGGACCCAG | |
| AACAGCCATATGAGCCCACTGGAAGGGAGTCGATCCCAGTCGCCGAGCGGAGACGACAC | |
| CAGCAGCAGCAGCGGCCACAGCAGTGTTGACACCATACCCTTCTTTAGCGAGAACCTCC | |
| CTATTGGTGAGCTGTTCTCCTATGTTGACCCCCTGACACACGCCCTATTCTCGGCTTGC | |
| ACTACGTTACATGTTGGGGTACAATTGCTGCGTGAGAATGAGATTACTCTGGGAGTACA | |
| CTCCGCCCAGGGTATTGCAGCTTCCATCAGCATGAGCGGGGAACCAGGCGAGGATATAG | |
| CCAGGACAGGGGCGACCAATTCCGCAAGATGCGAGGAGCAGCCGACCACTCCAGCGGCT | |
| CGGGTTTTGTTCATGTTCTTGAGTGATGAAGGGGCTTTCCAGGAGGGAAAGTCTGCTGG | |
| TTCCCGAGGTCGAACCATCGCAGCACTGCGACGATGCTATGAGGATATCTTTTCCCTCG | |
| CCCGCAAACACAAACATGGCATGCTCAGAGACCTCAACAATATTCCTCCATGA | |

TABLE 7
Amino Acid Sequence of lovE Variants at242 and

LovE at242

(SEQ ID NO:93)

mtqdtaqyrgamaadqgiftnsvtlspvegsrtggtlprrafrrscdrch
aqkikctgnkevtgrapcqrcqqaglrcvysercpkrklrqsraadlvsa
dpdpclhmssppvpsqslpldvseshssntsrqfldppdsydwswtsigt
deaidtdcwglsqcdggfscqleptlpdlpspfestvekaplppvssdia
raasaqrelfddlsaveqeleeillavtvewpkqeiwthpigmffnasrr
lltvlrqqaqadchqgtldeclrtknlftavhcyilnvriltaisellls
qirrtqnshmsplegsrsqspsrddtssssghssvdtipffsenlpigel
fsyvdplthalfsacttlhvgvqllreneitlgvhsaqgiaasismsgep
gediartgatnsarceeqpttpaarvlfmflsdegafqeaksagsrgrti
aalrrcyedifslarkhkhgmlrdlnnipp
LovE at242

(SEQ ID NO:94)

mlmtqdtaqyrgamaadqgiftnsvtlspvegsrtggtlprrafrrscdr
chaqkikctgnkevtgrapcqrcqqaglrcvysercpkrklrqsraadlv
sadpdpclhmssppvpsqslpldvseshssntsrqfldppdsydwswtsi
gtdeaidtdcwglsqcdggfscqleptlpdlpspfestvekaplppvssd
iaraasaqrelfddlsavsqeleeillavtvewpkqeiwthpigmffnas
rrlltvlrqqaqadchqgtldeclrtknlftavhcyilnvriltaisell
lsqirrtqnshmsplegsrsqspsrddtssssghssvdtipffsenlpig
elfsyvdplthalfsacttlhvgvqllreneitlgvhsaqgiaasismsg
epgediartgatnsarceeqpttpaarvlfmflsdegafqeaksagsrgr
tiaalrrcyedifslarkhkhgmlrdlnnipp

### Equivalents

Those skilled in the art will recognize, or be able to ascertain, using no more than routine experimentation, many equivalents to the specific embodiments of the invention described herein. Such equivalents are intended to be encompasssed by the following claims.

1. An isolated polypeptide comprising the amino acid sequence of SEQ ID NO:91 having an amino acid change selected from the group consisting of:
(a) a Phe changed to a Group 2 amino acid residue at position 31;
(b) a Gln changed to a Group 5 amino acid residue at position 41;
(c) a Thr changed to a Group 2 amino acid residue at position 52;
(d) a Thr changed to a Group 3 amino acid residue at position 52;
(e) a Cys changed to a Group 5 amino acid residue at position 73;
(f) a Pro changed to a Group 4 amino acid residue at position 101;
(g) a Pro changed to a Group 3 amino acid residue at position 101;
(h) a Val changed to a Group 2 amino acid residue other than Val at position 111;
(i) a Ser changed to a Group 2 amino acid residue at position 133;
(j) a Glu changed to a Group 2 amino acid residue at position 141;
(k) a Glu changed to a Group 5 amino acid residue at position 141;
(l) a Cys changed to a Group 6 amino acid residue at position 153;
(m) a Cys changed to a Group 5 amino acid residue at position 153;
(n) a Thr changed to a Group 1 amino acid residue at position 281;
(o) a Asn changed to a Group 2 amino acid residue at position 367;
(p) a Asn changed to a Group 6 amino acid residue at position 367;
(q) a Pro changed to a Group 4 amino acid residue at position 389; and
(r) a Pro changed to a Group 2 amino acid residue at position 389.

2. The polypeptide of claim 1 wherein the polypeptide when expressed in an A. terreus cell harboring a lovF gene increases expression of the lovF gene relative to an otherwise identical cell not expressing the polypeptide.

3. The polypeptide of claim 1 wherein the polypeptide when expressed in a S. cerevisiae harboring a gene under the control of the A. terreus lovF expression control region increases expression of the gene relative to an otherwise identical cell not expressing the polypeptide.

4. The isolated polypeptide of claim 1 having fewer than 11 amino acid changes.

5. The isolated polypeptide of claim 1 having fewer than 10 amino acid changes.

6. The isolated polypeptide of claim 1 having fewer than 8 amino acid changes.

7. The isolated polypeptide of claim 1 having fewer than 5 amino acid changes.

8. The isolated polypeptide of claim 1 wherein the polypeptide further comprises the amino acid sequence of SEQ ID NO:95 immediately amino terminal to the amino acid of SEQ ID NO:91.

9. The isolated polypeptide of claim 1 wherein the polypeptide further comprises the amino acid sequence of SEQ ID NO:96 immediately amino terminal to the amino acid of SEQ ID NO:91.

10. The isolated polypeptide of claim 1 having the amino acid change F31L.

11. The isolated polypeptide of claim 1 having the amino acid change Q41K or Q41R.

12. The isolated polypeptide of claim 1 having the amino acid change T52I.

13. The isolated polypeptide of claim 1 having the amino acid change T52N.

14. The isolated polypeptide of claim 1 having the amino acid change C73R.

15. The isolated polypeptide of claim 1 having the amino acid change P101S.

16. The isolated polypeptide of claim 1 having the amino acid change P101Q.

17. The isolated polypeptide of claim 1 having the amino acid change V111I.

18. The isolated polypeptide of claim 1 having the amino acid change S133L.

19. The isolated polypeptide of claim 1 having the amino acid change E141V.

20. The isolated polypeptide of claim 1 having the amino acid change E141K.

21. The isolated polypeptide of claim 1 having the amino acid change C153Y.

22. The isolated polypeptide of claim 1 having the amino acid change C153R.

23. The isolated polypeptide of claim 1 having the amino acid change T281A.

24. The isolated polypeptide of claim 1 having the amino acid change N367I.

25. The isolated polypeptide of claim 1 having the amino acid change N367Y.

26. The isolated polypeptide of claim 1 having the amino acid change P389S.

27. The isolated polypeptide of claim 1 having the amino acid change P389L.

28. The isolated polypeptide of claim 1 comprising an amino acid sequence selected from the group consisting of SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:91, SEQ ID NO:93, and SEQ ID NO:94.

29. An isolated nucleic acid molecule comprising a nucleotide sequence encoding a polypeptide comprising the amino acid sequence of SEQ ID NO:91 having at least one amino acid change selected from the group consisting of:
(a) a Phe changed to a Group 2 amino acid residue at position 31;
(b) a Gln changed to a Group 5 amino acid residue at position 41;
(c) a Thr changed to a Group 2 amino acid residue at position 52;
(d) a Thr changed to a Group 3 amino acid residue at position 52;
(e) a Cys changed to a Group 5 amino acid residue at position 73;
(f) a Pro changed to a Group 4 amino acid residue at position 101;
(g) a Pro changed to a Group 3 amino acid residue at position 101;
(h) a Val changed to a Group 2 amino acid residue other than Val at position 111;
(i) a Ser changed to a Group 2 amino acid residue at position 133;
(j) a Glu changed to a Group 2 amino acid residue at position 141;
(k) a Glu changed to a Group 5 amino acid residue at position 141;
(l) a Cys changed to a Group 6 amino acid residue at position 153;
(m) a Cys changed to a Group 5 amino acid residue at position 153;
(n) a Thr changed to a Group 1 amino acid residue at position 281;
(o) a Asn changed to a Group 2 amino acid residue at position 367;
(p) a Asn changed to a Group 6 amino acid residue at position 367;
(q) a Pro changed to a Group 4 amino acid residue at position 389; and
(r) a Pro changed to a Group 2 amino acid residue at position 389.

30. The isolated nucleic acid molecule of claim 29 wherein the polypeptide when expressed in an A. terreus cell harboring a lovF gene increases expression of the lovF gene relative to an otherwise identical cell not expressing the polypeptide.

31. The isolated nucleic acid molecule of claim 29 wherein the polypeptide when expressed in a S. cerevisiae harboring a gene under the control of the A. terreus lovF expression control region increases expression of the gene relative to an otherwise identical cell not expressing the polypeptide.

32. The isolated nucleic acid molecule of claim 29 wherein the polypeptide has fewer than 11 amino acid changes.

33. The isolated nucleic acid molecule of claim 29 wherein the polypeptide has fewer than 10 amino acid changes.

34. The isolated nucleic acid molecule of claim 29 wherein the polypeptide has fewer than 8 amino acid changes.

35. The isolated nucleic acid molecule of claim 29 wherein the polypeptide has fewer than 5 amino acid changes.

36. The isolated nucleic acid molecule of claim 29 wherein the polypeptide further comprises the amino acid sequence of SEQ ID NO:95 immediately amino terminal to the amino acid of SEQ ID NO:91.

37. The isolated nucleic acid molecule of claim 29 wherein the polypeptide further comprises the amino acid sequence of SEQ ID NO:96 immediately amino terminal to the amino acid of SEQ ID NO:91.

38. The isolated nucleic acid molecule of claim 29 wherein the polypeptide has the amino acid change F31L.

39. The isolated nucleic acid molecule of claim 29 wherein the polypeptide has the amino acid change Q41K or Q41R.

40. The isolated nucleic acid molecule of claim 29 wherein the polypeptide has the amino acid change T52I.

41. The isolated nucleic acid molecule of claim 29 wherein the polypeptide has the amino acid change T52N.

42. The isolated nucleic acid molecule of claim 29 wherein the polypeptide has the amino acid change C73R.

43. The isolated nucleic acid molecule of claim 29 wherein the polypeptide has the amino acid change P101S.

44. The isolated nucleic acid molecule of claim 29 wherein the polypeptide has the amino acid change P101Q.

45. The isolated nucleic acid molecule of claim 29 wherein the polypeptide has the amino acid change V111I.

46. The isolated nucleic acid molecule of claim 29 wherein the polypeptide has the amino acid change S133L.

47. The isolated nucleic acid molecule of claim 29 wherein the polypeptide has the amino acid change E141V.

48. The isolated nucleic acid molecule of claim 29 wherein the polypeptide has the amino acid change E141K.

49. The isolated nucleic acid molecule of claim 29 wherein the polypeptide has the amino acid change C153Y.

50. The isolated nucleic acid molecule of claim 29 wherein the polypeptide has the amino acid change C153R.

51. The isolated nucleic acid molecule of claim 29 wherein the polypeptide has the amino acid change T281A.

52. The isolated nucleic acid molecule of claim 29 wherein the polypeptide has the amino acid change N367I.

53. The isolated nucleic acid molecule of claim 29 wherein the polypeptide has the amino acid change N367Y.

54. The isolated nucleic acid molecule of claim 29 wherein the polypeptide has the amino acid change P389S.

55. The isolated nucleic acid molecule of claim 29 wherein the polypeptide has the amino acid change P389L.

56. The isolated nucleic acid molecule of claim 29 comprising a nucleotide sequence selected from the group consisting of: SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, and SEQ ID NO:90.

57. The isolated nucleic acid molecule of claim 29 wherein the nucleotide sequence encoding the polypeptide is contiguous.

58. A fungal cell containing a nucleic acid molecule encoding the polypeptide of claim 1.

59. A fungal cell containing the nucleic acid molecule off claim 29.

60. The fungal cell of claim 58 wherein the fungus is A. terreus.

61. A method for providing a fungal cell having improved production of a secondary metabolite, the method comprising transforming the fungal cell with a nucleic acid molecule of claim 1, whereby the fungal cell has increased secondary metabolite production compared to an otherwise identical fungal cell that has not been so transformed.

62. The method of claim 61 wherein the secondary metabolite is lovastatin.

63. A method for producing a secondary metabolite, the method comprising providing a fungal cell containing the nucleic acid molecule of claim 29, culturing the cell under conditions so as to produce the secondary metabolite, and isolating from the cells a fraction containing the secondary metabolite.

64. The method of claim 63 wherein the secondary metabolite is lovastatin.

65. An isolated polypeptide comprising the amino acid sequence of SEQ ID NO:91 having an amino acid change selected from the group consisting of: H253R, S341P, R121W, S322G, A83V, T135I, E177G, E197K, T281A, T256A, N466S, C73R, E303K, Q41K, Q41K, P16A, G223S, T9M, Q362E, R21H, S34A, Q80H, A84S, E303D, H374D, A440T, A441V, C445S, P469S, F31L, T409I, M971, E113D, D146N, P163S, H458Y, I43V, Q295L, F31L, C159S, E162K, R293L, S311N, L141, E18V, G138C, E338G, V361L, N400S, S174Y, A402T, F31L, P108S, D85N, I143F, M232I, T315I, S382Y, M385K, T461, Q62R, K77R, S323C, V373I, T2941, P310L, G337D, A394V, G436S, T139, V184I, D4E, V87I, D110E, A189T, N276D, T347R, N367I, Q377R, A425T, D131N, R312G, and A429G

66. The polypeptide of claim 65 wherein the polypeptide when expressed in an A. terreus cell harboring a lovF gene increases expression of the lovF gene relative to an otherwise identical cell not expressing the polypeptide.

67. The polypeptide of claim 65 wherein the polypeptide when expressed in a S. cerevisiae harboring a gene under the control of the A. terreus lovF expression control region increases expression of the gene relative to an otherwise identical cell not expressing the polypeptide.

68. The isolated polypeptide of claim 65 having fewer than 11 amino acid changes.

69. The isolated polypeptide of claim 65 having fewer than 10 amino acid changes.

70. The isolated polypeptide of claim 65 having fewer than 8 amino acid changes.

71. The isolated polypeptide of claim 65 having fewer than 5 amino acid changes.

72. The isolated polypeptide of claim 1 wherein the polypeptide has an amino acid sequence that is otherwise identical to SEQ ID NO:91.

73. The fungal cell of claim 59 wherein the fungus is A. terreus.

74. A method for providing a fungal cell having improved production of a secondary metabolite, the method comprising transforming the fungal cell with a nucleic acid molecule of claim 29, whereby the fungal cell has increased secondary metabolite production compared to an otherwise identical fungal cell that has not been so transformed.

75. The isolated polypeptide of claim 65 wherein the polypeptide has an amino acid sequence that is otherwise identical to SEQ ID NO:91.


SEQUENCE LISTING (96 sequences)

SEQ ID NO: 1 - DNA, 46 bp, Artificial Sequence
  primer
ggccatggaggccgctagctcgagtcgacggcctaggtggccagct

SEQ ID NO: 2 - DNA, 46 bp, Artificial Sequence
  primer
ggccacctaggccgtcgactcgagctagcggcctccatggccgtac

SEQ ID NO: 3 - DNA, 33 bp, Artificial Sequence
  primer
ggcggccgctctagaactagtctcgagggtacc

SEQ ID NO: 4 - DNA, 33 bp, Artificial Sequence
  primer
ggtaccctcgagactagttctagagcggccgcc

SEQ ID NO: 5 - DNA, 31 bp, Artificial Sequence
  primer
cacagcggccgctcaaccttcccattggggc

SEQ ID NO: 6 - DNA, 27 bp, Artificial Sequence
  primer
caccactagtacgcgggctgattcgac

SEQ ID NO: 7 - DNA, 33 bp, Artificial Sequence
  primer
caccactagttatacattatataaagtaatgtg

SEQ ID NO: 8 - DNA, 32 bp, Artificial Sequence
  primer
cacaggatccgtcatctttgccttcgtttatc

SEQ ID NO: 9 - DNA, 31 bp, Artificial Sequence
  primer
cgcggatcctattgaacaagatggattgcac

SEQ ID NO: 10 - DNA, 28 bp, Artificial Sequence
  primer
ccggaattcagaagaactcgtcaagaag

SEQ ID NO: 11 - DNA, 40 bp, Artificial Sequence
  primer
acaaaaaagcaggctccacaatggctgcagatcaaggtat

SEQ ID NO: 12 - DNA, 35 bp, Artificial Sequence
  primer
acaagaaagctgggttcatggaggaatattgttga

SEQ ID NO: 13 - DNA, 28 bp, Artificial Sequence
  primer
ggggatccaatcgaggtccacgaccagt

SEQ ID NO: 14 - DNA, 29 bp, Artificial Sequence
  primer
ggggacaagtttgtacaaaaaagcaggct

SEQ ID NO: 15 - DNA, 28 bp, Artificial Sequence
  primer
ggggatccgccaatggtcccgttcaaac

SEQ ID NO: 16 - DNA, 35 bp, Artificial Sequence
  primer
acaagaaagctgggttcacagaatgtttagctcaa

SEQ ID NO: 17 - DNA, 29 bp, Artificial Sequence
  primer
ggggaccactttgtacaagaaagctgggt

SEQ ID NO: 18 - DNA, 35 bp, Artificial Sequence
  primer
gcgatgccccaagcgcaagctacgccaatccaggg

SEQ ID NO: 19 - DNA, 35 bp, Artificial Sequence
  primer
cgtcgcgccattcgccattcaggctgcgcaactgt

SEQ ID NO: 20 - DNA, 31 bp, Artificial Sequence
  primer
ggacctttgcagcataaattactatacttct

SEQ ID NO: 21 - DNA, 35 bp, Artificial Sequence
  primer
ggcgcgtccattcgccattcaggctgcgcaactgt

SEQ ID NO: 22 - DNA, 31 bp, Artificial Sequence
  primer
taaaactcttgttttcttcttttctctaaat

SEQ ID NO: 23 - DNA, 36 bp, Artificial Sequence
  primer
cagtgagcgcgcgtaatacgactcactatagggcga

SEQ ID NO: 24 - DNA, 36 bp, Artificial Sequence
  primer
atacttctatagacacacaaacacaaatacacacac

SEQ ID NO: 25 - DNA, 23 bp, Artificial Sequence
  primer
cgcggatcccgtcgttttacaac

SEQ ID NO: 26 - DNA, 31 bp, Artificial Sequence
  primer
cccaagcttattatttttgacaccagaccaa

SEQ ID NO: 27 - DNA, 36 bp, Artificial Sequence
  primer
ggaagatctagcatcgtggccaatttcttctagttt

SEQ ID NO: 28 - DNA, 41 bp, Artificial Sequence
  primer
ataagaatgcggccgctcaaccttcccattggggcgtttgc

SEQ ID NO: 29 - DNA, 37 bp, Artificial Sequence
  primer
cacaggatccagcattattaatttagtgtgtgtattt

SEQ ID NO: 30 - DNA, 28 bp, Artificial Sequence
  primer
caccactagtctcgagcagatccgccag

SEQ ID NO: 31 - DNA, 27 bp, Artificial Sequence
  primer
caccactagtacgcgggctgattcgac

SEQ ID NO: 32 - DNA, 31 bp, Artificial Sequence; identical to SEQ ID NO: 5
  primer

SEQ ID NO: 33 - DNA, 35 bp, Artificial Sequence
  primer
ggccatcgatacaagtttgtacaaaaaagctgaac

SEQ ID NO: 34 - DNA, 33 bp, Artificial Sequence
  primer
ggcgccctattacaccactttgtacaagaaagc

SEQ ID NO: 35 - DNA, 35 bp, Artificial Sequence
  primer
cacacgtctccggcctcaaccttcccattggggcg

SEQ ID NO: 36 - DNA, 35 bp, Artificial Sequence
  primer
cacacagatctcgtggccaatttcttctagtttga

SEQ ID NO: 37 - DNA, 37 bp, Artificial Sequence
  primer
cacacggatccacaatgttacgtcctgtagaaacccc

SEQ ID NO: 38 - DNA, 34 bp, Artificial Sequence
  primer
cacagcggccgcttcattgtttgcctccctgctg

SEQ ID NO: 39 - DNA, 34 bp, Artificial Sequence
  primer
gcggccgcggcgcccggcccatgtcaacaagaat

SEQ ID NO: 40 - DNA, 25 bp, Artificial Sequence
  primer
ccgcggccgagtggagatgtggagt

SEQ ID NO: 41 - protein, 469 aa, Artificial Sequence
  synthetically generated variant
MAADQGIFTNSVTLSPVEGSRTGGTLPRRAFRRSCDRCHAQKIKCTGNKEVTGRAPCQRCQQAGLRCVYSERCPKRKLRQSRAADLVSADPDPCLHMSSPPVPSQSLPLDVSESHSSNTSRQFLDPPDSYDWSWTSIGTDEAIDTDCWGLSQCDGGFSCQLEPTLPDLPSPFESTVEKAPLPPVSSDIARAASAQRELFDDLSAVSQELEEILLAVTVEWPKQEIWTHPIGMFFNASRRLLTVLRQQAQADCRQGTLDECLRTKNLFTAVHCYILNVRILTAISELLLSQIRRTQNSHMSPLEGSRSQSPSRDDTSSSSGHSSVDTIPFFSENLPIGELFPYVDPLTHALFSACTTLHVGVQLLRENEITLGVHSAQGIAASISMSGEPGEDIARTGATNSARCEEQPTTPAARVLFMFLSDEGAFQEAKSAGSRGRTIAALRRCYEDIFSLARKHKHGMLRDLNNIPP

SEQ ID NO: 42 - protein, 469 aa, Artificial Sequence
  synthetically generated variant
MAADQGIFTNSVTLSPVEGSRTGGTLPRRAFRRSCDRCHAQKIKCTGNKEVTGRAPCQRCQQAGLRCVYSERCPKRKLRQSRAADLVSADPDPCLHMSSPPVPSQSLPLDVSESHSSNTSWQFLDPPDSYDWLWTSIGTDEAIDTDCWGLSQCDGGFSCQLEPTLPDLPSPFESTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRTQNSHMSPLEGSRSQSPSRDDTSSSSGHGSVDTIPFFSENLPIGELFSYVDPLTHALFSACTTLHVGVQLLRENEITLGVHSAQGIAASISMSGEPGEDIARTGATNSARCEEQPTTPAARVLFMFLSDEGAFQEAKSAGSRGRTIAALRRCYEDIFSLARKHKHGMLRDLNNIPP

SEQ ID NO: 43 - protein, 469 aa, Artificial Sequence
  synthetically generated variant
MAADQGIFTNSVTLSPVEGSRTGGTLPRRAFRRSCDRCHAQKIKCTGNKEVTGRAPCQRCQQAGLRCVYSERRPKRKLRQSRVADLVSADPDPCLHMSSPPVPSQSLPLDVSESHSSNTSRQFLDPPDSYDWSWISIGTDEAIDTDCWGLSQCDGGFSCQLEPTLPDLPSPFESTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRTQNSHMSPLEGSRSQSPSRDDTSSSSGHSSVDTIPFFSENLPIGELFSYVDPLTHALFSACTTLHVGVQLLRENEITLGVHSAQGIAASISMSGEPGEDIARTGATNSARCEEQPTTPAARVLFMFLSDEGAFQEAKSAGSRGRTIAALRRCYEDIFSLARKHKHGMLRDLNNIPP

SEQ ID NO: 44 - protein, 469 aa, Artificial Sequence
  synthetically generated variant
MAADQGIFTNSVTLSPVEGSRTGGTLPRRAFRRSCDRCHAQKIKCTGNKEVTGRAPCQRCQQAGLRCVYSERRPKRKLRQSRAADLVSADPDPCLHMSSPPVPSQSLPLDVSESHSSNTSRQFLDPPDSYDWSWTSIGTDEAIDTDCWGLSQCDGGFSCQLEPTLPDLPSPFESTVGKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRTQNSHMSPLEGSRSQSPSRDDTSSSSGHSSVDTIPFFSENLPIGELFSYVDPLTHALFSACTTLHVGVQLLRENEITLGVHSAQGIAASISMSGEPGEDIARTGATNSARCEEQPTTPAARVLFMFLSDEGAFQEAKSAGSRGRTIAALRRCYEDIFSLARKHKHGMLRDLNNIPP

SEQ ID NO: 45 - protein, 469 aa, Artificial Sequence
  synthetically generated variant
MAADQGIFTNSVTLSPVEGSRTGGTLPRRAFRRSCDRCHAQKIKCTGNKEVTGRAPCQRCQQAGLRCVYSERRPKRKLRQSRAADLVSADPDPCLHMSSPPVPSQSLPLDVSESHSSNTSRQFLDPPDSYDWSWTSIGTDEAIDTDCWGLSQCDGGFSCQLEPTLPDLPSPFESTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRTQNSHMSPLEGSRSQSPSRDDTSSSSGHSSVDTIPFFSENLPIGELFSYVDPLTHALFSACTTLHVGVQLLRENEITLGVHSAQGIAASISMSGEPGEDIARTGATNSARCEEQPTTPAARVLFMFLSDEGAFQEAKSAGSRGRTIAALRRCYEDIFSLARKHKHGMLRDLNNIPP

SEQ ID NO: 46 - protein, 469 aa, Artificial Sequence
  synthetically generated variant
MAADQGIFTNSVTLSPVEGSRTGGTLPRRAFRRSCDRCHAQKIKCTGNKEVTGRAPCQRCQQAGLRCVYSERCPKRKLRQSRAADLVSADPDPCLHMSSPPVPSQSLPLDVSESHSSNTSRQFLDPPDSYDWSWTSIGTDEAIDTDCWGLSQYDGGFSCQLEPTLPDLPSPFESTVEKAPLPPVSSDIARAASAQRKLFDDLSAVSQELEEILLAVTVEWPKQEIWTHPIGMFFNASRRLLTVLRQQAQADCHQGTLDECLRTKNLFTAVHCYILNVRILAAISELLLSQIRRTQNSHMSPLEGSRSQSPSRDDTSSSSGHSSVDTIPFFSENLPIGELFSYVDPLTHALFSACTTLHVGVQLLRENEITLGVHSAQGIAASISMSGEPGEDIARTGATNSARCEEQPTTPAARVLFMFLSDEGAFQEAKSAGSRGRTIAALRRCYEDIFSLARKHKHGMLRDLNNIPP

SEQ ID NO: 47 - protein, 469 aa, Artificial Sequence
  synthetically generated variant
MAADQGIFTNSVTLSPVEGSRTGGTLPRRAFRRSCDRCHAQKIKCTGNKEVTGRAPCQRCQQAGLRCVYSERRPKRKLRQSRAADLVSADPDPCLHMSSPPVPSQSLPLDVSESHSSNTSRQFLDPPDSYDWSWTSIGTDEAIDTDCWGLSQCDGGFSCQLEPTLPDLPSPFESTVEKAPLPPVSSDIARAASAQRELFDDLSAVSQELEEILLAVTVEWPKQEIWTHPIGMFFNASRRLLTVLRQQAQADCHQGALDECLRTKNLFTAVHCYILNVRILTAISELLLSQIRRTQNSHMSPLEGSRSQSPSRDDTSSSSGHSSVDTIPFFSENLPIGELFSYVDPLTHALFSACTTLHVGVQLLRENEITLGVHSAQGIAASISMSGEPGEDIARTGATNSARCEEQPTTPAARVLFMFLSDEGAFQEAKSAGSRGRTIAALRRCYEDIFSLARKHKHGMLRDLNSIPP

SEQ ID NO: 48 - protein, 469 aa, Artificial Sequence; identical to SEQ ID NO: 47
  synthetically generated variant

SEQ ID NO: 49 - protein, 469 aa, Artificial Sequence; identical to SEQ ID NO: 47
  synthetically generated variant

SEQ ID NO: 50 - protein, 469 aa, Artificial Sequence; identical to SEQ ID NO: 47
  synthetically generated variant

SEQ ID NO: 51 - protein, 469 aa, Artificial Sequence
  synthetically generated variant
MAADQGIFMNSVTLSAVEGSRTSGTLPRRAFRRSCDRCHAKKIKCTGNKEVTGRAPCQRCQQAGLRCVYSERCPKRKLRQSRAADLVSADPDPCLHMSSPPVPSQSLPLDVSESHSSNTSRQFLDPPDSYDWSWTSIGTDEAIDTDCWGLSQCDGGFSCQLEPTLPDLPSPFESTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRTQNSHMSPLEGSRSQSPSRDDTSSSSGHSSVDTIPFFSENLPIGELFSYVDPLTHALFSACTTLHVGVELLRENEITLGVHSAQGIAASISMSGEPGEDIARTGATNSARCEEQPTTPAARVLFMFLSDEGAFQEAKSAGSRGRTIAALRRCYEDIFSLARKHKHGMLRDLNNIPP

SEQ ID NO: 52 - protein, 469 aa, Artificial Sequence
  synthetically generated variant
MAADQGIFTNSVTLSPVEGSHTGGTLPRRAFRRACDRCHAQKIKCTGNKEVTGRAPCQRCQQAGLRCVYSERCPKRKLRHSRASDLVSADPDPCLHMSSPPVPSQSLPLDVSESHSSNTSRQFLDPPDSYDWSWTSIGTDEAIDTDCWGLSQCDGGFSCQLEPTLPDLPSPFESTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRTQNSHMSPLDGSRSQSPSRDDTSSSSGHSSVDTIPFFSENLPIGELFSYVDPLTHALFSACTTLHVGVQLLRENEITLGVDSAQGIAASISMSGEPGEDIARTGATNSARCEEQPTTPAARVLFMFLSDEGAFQEAKSAGSRGRTITVLRRSYEDIFSLARKHKHGMLRDLNNIPS

SEQ ID NO: 53 - protein, 469 aa, Artificial Sequence
  synthetically generated variant
MAADQGIFTNSVTLSPVEGSRTGGTLPRRALRRSCDRCHAQKIKCTGNKEVTGRAPCQRCQQAGLRCVYSERCPKRKLRQSRAADLVSADPDPCLHMSSPPVPSQSLPLDVSESHSSNTSRQFLDPPDSYDWSWTSIGTDEAIDTDCWGLSQCDGGFSCQLEPTLPDLPSPFESTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRTQNSHMSPLEGSRSQSPSRDDTSSSSGHSSVDTIPFFSENLPIGELFSYVDPLTHALFSACTTLHVGVQLLRENEITLGVHSAQGIAASISMSGEPGEDIARTGATNSARCEEQPITPAARVLFMFLSDEGAFQEAKSAGSRGRTIAALRRCYEDIFSLARKHKHGMLRDLNNIPP

SEQ ID NO: 54 - protein, 469 aa, Artificial Sequence
  synthetically generated variant
MAADQGIFTNSVTLSPVEGSRTGGTLPRRALRRSCDRCHAQKIKCTGNKEVTGRAPCQRCQQAGLRCVYSERCPKRKLRQSRAADLVSADPDPCLHISSPPVPSQSLPLDVSDSHSSNTSRQFLDPPDSYDWSWTSIGTDEAIDTNCWGLSQCDGGFSCQLESTLPDLPSPFESTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRTQNSHMSPLEGSRSQSPSRDDTSSSSGHSSVDTIPFFSENLPIGELFSYVDPLTHALFSACTTLHVGVQLLREIEITLGVHSAQGIAASISMSGEPGEDIARTGATNSARCEEQPTTPAARVLFMFLSDEGAFQEAKSAGSRGRTIAALRRCYEDIFSLARKHKYGMLRDLNNIPP

SEQ ID NO: 55 - protein, 470 aa, Artificial Sequence
  synthetically generated variant
MAADQGIFTNSVTLSPVEGSRTGGTLPRRAFRRSCDRCHAQKVKCTGNKEVTGRAPCQRCQQAGLRCVYSERCPKRKLRQSRAADLVSADPDPCLHMSSPPVPSQSLPLDVSESHSSNTSRQFLDPPDSYDWSWTSIGTDEAIDTDCWGLSQCDGGFSCQLEPTLPDLPSPFESTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRTLNSHMSPLEGSRSQSPSRDDTSSSSGHSSVDTIPFFSENLPIGELFSYVDPLTHALFSACTTLHVGVQLLRENEITLGVHSAQGIAASISMSGEPGEDIARTGATNSARCEEQPTTPAARVLFMFLSDEGAFQEAKSAGSRGRTIAALRRCYEDIFSLARKHKHGMLRDLNNIPPC

SEQ ID NO: 56 - protein, 469 aa, Artificial Sequence
  synthetically generated variant
MAADQGIFTNSVTLSPVEGSRTGGTLPRRALRRSCDRCHAQKIKCTGNKEVTGRAPCQRCQQAGLRCVYSERCPKRKLRQSRAADLVSADPDPCLHMSSPSVPSQSLPLDVSESHSSNTSRQFLDPPDSYDWSWTSIGTDEAIDTDCWGLSQRDGGFSSQLKPTLPDLPSPFESTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRLTQNSHMSPLEGSRSQSPNRDDTSSSSGHSSVDTIPFFSENLPIGELFSYVDPLTHALFSACTTLHVGVQLLRENEITLGVHSAQGIAASISMSGEPGEDIARTGATNSARCEEQPTTPAARVLFMFLSDEGAFQEAKSAGSRGRTIAALRRCYEDIFSLARKHKHGMLRDLNNIPP

SEQ ID NO: 57 - protein, 469 aa, Artificial Sequence
  synthetically generated variant
MAADQGIFTNSVTISPVVGSRTGGTLPRRAFRRSCDRCHAQKIKCTGNKEVTGRAPCQRCQQAGLRCVYSERCPKRKLRQSRAADLVSADPDPCLHMSSPPVPSQSLPLDVSESHSSNTSRQFLDPPDSYDWSWTSICTDEAIDTDCWGLSQCDGGFSCQLEPTLPDLPSPFESTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRTQNSHMSPLEGSRSQSPSRDDTSSSSGHSSVDTIPFFSENLPIGGLFSYVDPLTHALFSACTTLHVGLQLLRENEITLGVHSAQGIAASISMSGESGEDIARTGATSSARCEEQPTTPAARVLFMFLSDEGAFQEAKSAGSRGRTIAALRRCYEDIFSLARKHKHGMLRDLNNIPP

SEQ ID NO: 58 - protein, 469 aa, Artificial Sequence
  synthetically generated variant
MAADQGIFTNSVTLSPVEGSRTGGTLPRRAFRRSCDRCHARKIKCTGNKEVTGRAPCQRCQQAGLRCVYSERCPKRKLRQSRAADLVSADPDPCLHMSSPPVPSQSLPLDVSESHSSNTSRQFLDPPDSYDWSWTSIGTDEAIDTDCWGLSQCDGGFSCQLEPTLPDLPSPFEYTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRTQNSHMSPLEGSRSQSPSRDDTSSSSGHSSVDTIPFFSENLPIGELFSYVDPLTHALFSACTTLHVGVQLLRENEITLGVHSAQGIAASISMSGEPGEDIARTGATNSTRCEEQPTTPAARVLFMFLSDEGAFQEAKSAGSRGRTIAALRRCYEDIFSLARKHKHGMLRDLNNIPP

SEQ ID NO: 59 - protein, 469 aa, Artificial Sequence
  synthetically generated variant
MAADQGIFTNSVTLSPVEGSRTGGTLPRRALRRSCDRCHAQKIKCTGNKEVIGRAPCQRCQQAGLRCVYSERCPKRKLRQSRAADLVSADPDPCLHMSSPQVPSQSLSLDISESHSSNTSRQFLDPPDSYDWSWTSIGTDEAIDTDCWGLSQCDGGFSCQLEPTLPDLPSPFESTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRTQNSHMSPLEGSRSQSPSRDDTSSSSGHSSVDTIPFFSENLPIGELFSYVDPLTHALFSACTTLHVGVQLLRENEITLGVHSAQGIAASISMSGEPGEDIARTGATNSARCEEQPTTPAARVLFMFLSDEGAFQEAKSAGSRGRTIAALRRCYEDIFSLARKHKHGMLRDLNNIPP

SEQ ID NO: 60 - protein, 469 aa, Artificial Sequence
  synthetically generated variant
MAADQGIFTNSVTLSPVEGSRTGGTLPRRAFRRSCDRCHAQKIKCTGNKEVTGRAPCQRCQQAGLRCVYSERCPKRKLRQSRAANLVSADPDPCLHMSSPPVPSQSLPLDVSESHSSNTSRQFLDPPDSYDWSWTSIGTDEAFDTDCWGLSQCDGGFSCQLEPTLPDLPSPFESTVEKAPLPPVSSDIARAASAQRELFDDLSAVSQELEEILLAVTVEWPKQEIWTHPIGIFFNASRRLLTVLRQQAQADCHQGTLDECLRTKNLFTAVHCYILNVRILTAISELLLSQIRRTQNSHMSPLEGSRSQSPSRDDISSSSGHSSVDTIPFFSENLPIGELFSYVDPLTHALFSACTTLHVGVQLLRENEITLGVHSAQGIAAYISKSGEPGEDIARTGATNSARCEEQPTTPAARVLFMFLSDEGAFQEAKSAGSRGRTIAALRRCYEDIFSLARKHKHGMLRDLNNIPP

SEQ ID NO: 61 - protein, 469 aa, Artificial Sequence
  synthetically generated variant
MAADQGIFTNSVTLSPVEGSRTGGTLPRRAFRRSCDRCHAQKIKCIGNKEVTGRAPCQRCQRAGLRCVYSERCPKRRLRQSRAADLVSADPDPCLHMSSPPVPSQSLPLDVSESHSSNTSRQFLDPPDSYDWSWTSIGTDEAIDTDCWGLSQCDGGFSCQLEPTLPDLPSPFESTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRTQNSHMSPLEGSRSQSPSRDDTSSSSGHSCVDTIPFFSENLPIGELFSYVDPLTHALFSACTTLHVGVQLLREYEITLGIHSAQGIAASISMSGEPGEDIARTGATNSARCEEQPTTPAARVLFMFLSDEGAFQEAKSAGSRGRTIAALRRCYEDIFSLARKHKHGMLRDLNNIPP

SEQ ID NO: 62 - protein, 469 aa, Artificial Sequence
  synthetically generated variant
MAADQGIFTNSVTLSPVEGSRTGGTLPRRAFRRSCDRCHARKIKCTGNKEVTGRAPCQRCQQAGLRCVYSERCPKRKLRQSRAADLVSADPDPCLHMSSPPVPSQSLPLDVSESHSSNTSRQFLDPPDSYDWSWTSIGTDEAIDTDCWGLSQCDGGFSCQLEPTLPDLPSPFESTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRIQNSHMSPLEGSRSQSLSRDDTSSSSGHSSVDTIPFFSENLPIDELFSYVDPLTHALFSACTTLHVGVQLLRENEITLGVHSAQGIAASISMSGELGEDIVRTGATNSARCEEQPTTPAARVLFMFLSDEGAFQEAKSAGSRSRTIAALRRCYEDIFSLARKHKHGMLRDLNNIPP

SEQ ID NO: 63 - protein, 469 aa, Artificial Sequence
  synthetically generated variant
MAADQGIFTNSVTLSPVEGSRTGGTLPRRAFRRSCDRCHAQKIKCTGNKEVNGRAPCQRCQQAGLRCVYSERCPKRKLRQSRAADLVSADPDPCLHMSSPPVPSQSLPLDISESHSSNTSRQFLDPPDSYDWSWTSIGIDEAIDTDCWGLSQCDGGFSCQLEPTLPDLPSPFESTVEKAPLPPISSDIARAASAQRELFDDLSAVSQELEEILLAVTVEWPKQEIWTHPIGMFFNASRRLLTVLRQQAQADCHQGTLDECLRTKNLFTAVHCYILNVRILAAISELLLSQIRRTQNSHMSPLEGSRSQSPSRDDTSSSSGHSSVDTIPFFSENLPIGELFSYVDPLTHALFSACTTLHVGVQLLRENEITLGVHSAQGIAASISMSGEPGEDIARTGATNSARCEEQPTTPAARVLFMFLSDEGAFQEAKSAGSRGRTIAALRRCYEDIFSLARKHKHGMLRDLNNIPP

SEQ ID NO: 64 - protein, 469 aa, Artificial Sequence
  synthetically generated variant
MAAEQGIFTNSVTLSPVEGSRTGGTLPRRAFRRSCDRCHARKIKCTGNKEVTGRAPCQRCQQAGLRCVYSERCPKRKLRQSRAADLISADPDPCLHMSSPPVPSQSLPLEVSESHSSNTSRQFLDPPDSYDWSWTSIGTDKAIDTDCWGLSQCDGGFSCQLEPTLPDLPSPFESTVEKAPLPPVSSDITRAASAQRELFDDLSAVSQELEEILLAVTVEWPKQEIWTHPIGMFFNASRRLLTVLRQQAQADCHQGTLDECLRTKNLFTAVHCYILDVRILTAISELLLSQIRRTQNSHMSPLEGSRSQSPSRDDTSSSSGHSSVDTIPFFSENLPIGELFSYVDPLRHALFSACTTLHVGVQLLREIEITLGVHSARGIAASISMSGEPGEDIARTGATNSARCEEQPTTPAARVLFMFLSDEGTFQEAKSAGSRGRTIAALRRCYEDIFSLARKHKHGMLRDLNNIPP

SEQ ID NO: 65 - protein, 469 aa, Artificial Sequence
  synthetically generated variant
MAADQGIFTNSVTLSPVEGSRTGGTLPRRAFRRSCDRCHAQKIKCTGNKEVTGRAPCQRCQQAGLRCVYSERCPKRKLRQSRAADLVSADPDPCLHMSSPPVPSQSLPLDVSESHSSNTSRQFLDPPDSYNWLWTSIGTDEAIDTDCWGLSQCDGGFSCQLEPTLPDLPSPFESTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRTQNSHMSPLEGSRSQSPSGDDTSSSSGHSSVDTIPFFSENLPIGELFSYVDPLTHALFSACTTLHVGVQLLRENEITLGVHSAQGIAASISMSGEPGEDIARTGATNSARCEEQPTTPAARVLFMFLSDEGAFQEGKSAGSRGRTIAALRRCYEDIFSLARKHKHGMLRDLNNIPP

SEQ ID NO: 66 - DNA, 1410 bp, Artificial Sequence
  synthetically generated variant
atggctgcagatcaaggtatattcacgaactcggtcactctctcgccagtggagggttcacgcaccggtggaacattaccccgccgtgcattccgacgctcttgtgatcggtgtcatgcacaaaagatcaaatgtactggaaataaggaggttactggccgtgctccctgtcagcgttgccagcaggctggacttcgatgcgtctacagtgagcgatgccccaagcgcaagctacgccaatccagggcagcggatctcgtctctgctgacccagatccctgcttgcacatgtcctcgcctccagtgccctcacagagcttgccgctagacgtatccgagtcgcattcctcaaatacctcccggcagtttcttgatccaccggacagctacgactggtcgtggacctcgattggcactgacgaggctattgacactgactgctgggggctgtcccaatgtgatggaggcttcagctgtcagttagagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccgtcaaggcacactagacgaatgtttacggaccaagaacctctttacggcagtacactgttacatattgaatgtgcggattttgaccgccatatcggagttgctcctgtcgcaaattaggcggacccagaacagccatatgagcccactggaagggagtcgatcccagtcgccgagcagagacgacaccagcagcagcagcggccacagcagtgttgacaccatacccttctttagcgagaacctccctattggtgagctgttcccctatgttgaccccctgacacacgccctattctcggcttgcactacgttacatgttggggtacaattgctgcgtgagaatgagattactctgggagtacactccgcccagggcattgcagcttccatcagcatgagcggggaaccaggcgaggatatagccaggacaggggcgaccaattccgcaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 67 - DNA, 1410 bp, Artificial Sequence
  synthetically generated variant
atggctgcagatcaaggtatattcacgaactcggtcactctctcgccagtggagggttcacgcaccggtggaacattaccccgccgtgcattccgacgctcttgtgatcggtgtcatgcacaaaagatcaaatgtactggaaataaggaggttactggccgtgctccctgtcagcgttgccagcaggctggacttcgatgcgtctacagtgagcgatgccccaagcgcaagctacgccaatccagggcagcggatctcgtctctgctgacccagatccctgcttgcacatgtcctcgcctccagtgccctcacagagcttgccgctagacgtatccgagtcgcattcctcaaatacctcctggcaatttcttgatccaccggacagctacgactggttgtggacctcgattggcactgacgaggctattgacactgactgctgggggctgtcccaatgtgatggaggcttcagctgtcagttagagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagagtggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctcctgtcgcaaattaggcggacccagaacagccatatgagcccactggaagggagtcgatcccagtcgccgagcagagacgacaccagcagcagcagcggccacggcagtgttgacaccatacccttctttagcgagaacctccctattggtgagctgttctcctatgttgaccccctgacacacgccctattctcggcttgcactacgttacatgttggggtacaattgctgcgtgagaatgagattactctgggagtacactccgcccagggcattgcagcttccatcagcatgagcggggaaccaggcgaggatatagccaggacaggggcgaccaattccgcaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 68 - DNA, 1410 bp, Artificial Sequence
  synthetically generated variant
atggctgcagatcaaggtatattcacgaactcggtcactctctcgccagtggagggttcacgcaccggtggaacattaccccgccgtgcattccgacgctcttgtgatcggtgtcatgcacaaaagatcaaatgtactggaaataaggaggttactggccgtgctccctgtcagcgttgccagcaggctggacttcgatgcgtctacagtgagcgacgccccaagcgcaagctacgccaatccagggtagcggatctcgtctctgctgacccagatccctgcttgcacatgtcctcgcctccagtgccctcacagagcttgccgctagacgtatccgagtcgcattcctcaaatacctcccggcaatttcttgatccaccggacagctacgactggtcgtggatctcgattggcactgacgaggctattgacactgactgctgggggctgtcccaatgtgatggaggcttcagctgtcagttagagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctcctgtcgcaaattaggcggacccagaacagccatatgagcccactggaagggagtcgatcccagtcgccgagcagagacgacaccagcagcagcagcggccacagcagtgttgacaccatacccttctttagcgagaacctccctattggtgagctgttctcctatgttgaccccctgacacacgccctattctcggcttgcactacgttacatgttggggtacaattgctgcgtgagaatgagattactctgggagtacactccgcccagggcattgcagcttccatcagcatgagcggggaaccaggcgaggatatagccaggacaggggcgaccaattccgcaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 69 - DNA, 1410 bp, Artificial Sequence
  synthetically generated variant
atggctgcagatcaaggtatattcacgaactcggtcactctctcgccagtggagggttcacgcaccggtggaacattaccccgccgtgcattccgacgctcttgtgatcggtgtcatgcacaaaagatcaaatgtactggaaataaggaggttactggccgtgctccctgtcagcgttgccagcaggctggacttcgatgcgtctacagtgagcgacgccccaagcgcaagctacgccaatccagggcagcggatctcgtctctgctgacccagatccctgcttgcacatgtcctcgcctccagtgccctcacagagcttgccgctagacgtatccgagtcgcattcctcaaatacctcccggcaatttcttgatccaccggacagctacgactggtcgtggacctcgattggcactgacgaggctattgacactgactgctgggggctgtcccaatgtgatggaggcttcagctgtcagttagagccaacgctgccggatctaccttcgcccttcgagtctacggttggaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctcctgtcgcaaattaggcggacccagaacagccatatgagcccactggaagggagtcgatcccagtcgccgagcagagacgacaccagcagcagcagcggccacagcagtgttgacaccatacccttctttagcgagaacctccctattggtgagctgttctcctatgttgaccccctgacacacgccctattctcggcttgcactacgttacatgttggggtacaattgctgcgtgagaatgagattactctgggagtacactccgcccagggcattgcagcttccatcagcatgagcggggaaccaggcgaggatatagccaggacaggggcgaccaattccgcaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 70 - DNA, 1410 bp, Artificial Sequence
  synthetically generated variant
atggctgcagatcaaggtatattcacgaactcggtcactctctcgccagtggagggttcacgcaccggtggaacattaccccgccgtgcattccgacgctcttgtgatcggtgtcatgcacaaaagatcaaatgtactggaaataaggaggttactggccgtgctccctgtcagcgttgccagcaggctggacttcgatgcgtctacagtgagcgacgccccaagcgcaagctacgccaatccagggcagcggatctcgtctctgctgacccagatccctgcttgcacatgtcctcgcctccagtgccctcacagagcttgccgctagacgtatccgagtcgcattcctcaaatacctcccggcaatttcttgatccaccggacagctacgactggtcgtggacctcgattggcactgacgaggctattgacactgactgctgggggctgtcccaatgtgatggaggcttcagctgtcagttagagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctcctgtcgcaaattaggcggacccagaacagccatatgagcccactggaagggagtcgatcccagtcgccgagcagagacgacaccagcagcagcagcggccacagcagtgttgacaccatacccttctttagcgagaacctccctattggtgagctgttctcctatgttgaccccctgacacacgccctattctcggcttgcactacgttacatgttggggtacaattgctgcgtgagaatgagattactctgggagtacactccgcccagggcattgcagcttccatcagcatgagcggggaaccaggcgaggatatagccaggacaggggcgaccaattccgcaagatgcgaggagcagccgactactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 71 - DNA, 1410 bp, Artificial Sequence
  synthetically generated variant
atggctgcagatcaaggtatattcacgaactcggtcactctctcgccagtggagggttcacgcaccggtggaacattaccccgccgtgcattccgacgctcttgtgatcggtgtcatgcacaaaagatcaaatgtactggaaataaggaggttactggccgtgctccctgtcagcgttgccagcaggctggacttcgatgcgtctacagtgagcgatgccccaagcgcaagctacgccaatccagggcagcggatctcgtctctgctgacccagatccctgcttgcacatgtcctcgcctccagtgccctcacagagcttgccgctagacgtatccgagtcgcattcctcaaatacctcccggcaatttcttgatccaccggacagctacgactggtcgtggacctcgattggcactgacgaggctattgacactgactgctgggggctgtcccaatatgatggaggcttcagctgtcagttagagccaacgctgccggatctaccttcgcccttcgagtctacggttgaaaaagctccgttgccaccggtatcgagcgacattgctcgtgcggccagtgcgcaacgaaagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttggccgccatatcggagttgctcctgtcgcaaattaggcggacccagaacagccatatgagcccactggaagggagtcgatcccagtcgccgagcagagacgacaccagcagcagcagcggccacagcagtgttgacaccatacccttctttagcgagaacctccctattggtgagctgttctcctatgttgaccccctgacacacgccctattctcggcttgcactacgttacatgttggggtacaattgctgcgtgagaatgagattactctgggagtacactccgcccagggcattgcagcttccatcagcatgagcggggaaccaggcgaggatatagccaggacaggggcgaccaattccgcaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 72 - DNA, 1410 bp, Artificial Sequence
  synthetically generated variant
atggctgcagatcaaggtatattcacgaactcggtcactctctcgccagtggagggttcacgcaccggtggaacattaccccgccgtgcattccgacgctcttgtgatcggtgtcatgcacaaaagatcaaatgtactggaaataaggaggttactggccgtgctccctgtcagcgttgccagcaggctggacttcgatgcgtctacagtgagcgacgccccaagcgcaagctacgccaatccagggcagcggatctcgtctctgctgacccagatccctgcttgcacatgtcctcgcctccagtgccctcacagagcttgccgctagacgtatccgagtcgcattcctcaaatacctcccggcaatttcttgatccaccggacagctacgactggtcgtggacctcgattggcactgacgaggctattgacactgactgctgggggctgtcccaatgtgatggaggcttcagctgtcagttagagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcgcactagacgaatgtttacggaccaagaacctctttacggcagtacactgttacatattgaatgtgcggattttgaccgccatatcggagttgctcctgtcgcaaattaggcggacccagaacagccatatgagcccactggaagggagtcgatcccagtcgccgagcagagacgacaccagcagcagcagcggccacagcagtgttgacaccatacccttctttagcgagaacctccctattggtgagctgttctcctatgttgaccccctgacacacgccctattctcggcttgcactacgttacatgttggggtacaattgctgcgtgagaatgagattactctgggagtacactccgcccagggcattgcagcttccatcagcatgagcggggaaccaggcgaggatatagccaggacaggggcgaccaattccgcaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacagtattcctccatga

SEQ ID NO: 73 - DNA, 1410 bp, Artificial Sequence
  synthetically generated variant
atggctgcagatcaaggtatattcacgaactcggtcactctctcgccagtggagggttcacgcaccggtggaacattaccccgccgtgcattccgacgctcttgtgatcggtgtcatgcacaaaagatcaaatgtactggaaataaggaggttactggccgtgctccctgtcagcgttgccagcaggctggacttcgatgcgtctacagtgagcgacgccccaagcgcaagctacgccaatccagggcagcggatctcgtctctgctgacccagatccctgcttgcacatgtcctcgcctccagtgccctcacagagcttgccgctagacgtatccgagtcgcattcctcaaatacctcccggcaatttcttgatccaccggacagctacgactggtcgtggacctcgattggcactgacgtggctattgacactgactgctgggggctgtcccaatgtgatggaggcttcagctgtcagttagagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctcctgtcgcaaattaggcggacccagaacagccatatgagcccactggaagggagtcgatcccagtcgccgagcagagacgacaccagcagcagcagcggccacagcagtgttgacaccatacccttctttagcgagaacctccctattggtgagctgttctcctatgttgaccccctgacacacgccctattctcggcttgcactacgttacatgttggggtacaattgctgcgtgagaatgagattactctgggagtacactccgcccagggcattgcagcttccatcagcatgagcggggaaccaggcgaggatatagccaggacaggggcgaccaattccgcaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 74 - DNA, 1410 bp, Artificial Sequence
  synthetically generated variant
atggctgcagatcaaggtatattcacgaactcggtcactctctcgccagtggagggttcacgcaccggtggaacattaccccgccgtgcattccgacgctcttgtgatcggtgtcatgcacaaaagatcaaatgtactggaaataaggaggttactggccgtgctccctgtcagcgttgccagcaggctggacttcgatgcgtctacagtgagcgacgccccaagcgcaagctacgccaatccagggcagcggatctcgtttctgctgacccagatccctgcttgcacatgtcctcgcctccagtgccctcacagagcttgccactagacgtatccgagtcgcattcctcaaatacctcccggcaatttcttgatccaccggacagctacgactggtcgtggacctcgattggcactgacgaggctattgacactgactgctgggggctgtcccaatgtgatggaggcttcagctgtcagttagagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaacgtgcggattttgaccgccatatcggagttgctcctgtcgcaaattaggcggacccagaacagccatatgagcccactgaaagggagtcgatcccagtcgccgagcagagacgacaccagcagcagcagcggccacagcagtgttgacaccatacccttctttagcgagaacctccctattggtgagctgttctcctatgttgaccccctgacacacgccctattctcggcttgcactacgttacatgttggggtacaattgctgcgtgagaatgagattactctgggagtacactccgcccagggcattgcagcttccatcagcatgagcggggaaccaggcgaggatatagccaggacaggggcgaccaattccgcaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 75 - DNA, 1410 bp, Artificial Sequence
  synthetically generated variant
atggctgcagatcaaggtatattcacgaactcggtcactctctcgccagtggagggttcacgcaccggtggaacattaccccgccgtgcattccgacgctcttgtgatcggtgtcatgcaaaaaagatcaaatgtactggaaataaggaggttactggccgtgctccctgtcagcgttgccagcaggctggacttcgatgcgtctacagtgagcgatgccccaagcgcaagctacgccaatccagggcagcggatctcgtctctgctgacccagatccctgcttgcacatgtcctcgcctccagtgccctcacagagcttgccgctagacgtatccgagtcgcattcctcaaatacctcccggcaatttcttgatccaccggacagctacgactggtcgtggacctcgattggcactgacgaggctattgacactgactgctgggggctgtcccaatgtgatggaggcttcagctgtcagttagagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctcctgtcgcaaattaggcggacccagaacagccatatgagcccactggaagggagtcgatcccagtcgccgagcagagacgacaccagcagcagcagcggccacagcagtgttgacaccatacccttctttagcgagaacctccctattggtgagctgttctcctatgttgaccccctgacacacgccctattctcggcttgcactacgctacatgttggggtacaattgctgcgtgagaatgagattactctgggagtacactccgcccagggcattgcagcttccatcagcatgagcggggaaccaggcgaggatatagccaggacaggggcgaccaattccgcaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 76 - DNA, 1410 bp, Artificial Sequence
  synthetically generated variant
atggctgcagatcaaggtatattcatgaactcggtcactctctctgcagtggagggttcacgcaccagtggaacattaccccgccgtgcattccgacgctcttgtgatcggtgtcatgcaaaaaagatcaaatgtactggaaataaggaggttactggccgtgctccctgtcagcgttgccagcaggctggacttcgatgcgtctacagtgagcgatgccccaagcgcaagctacgccaatccagggcagcggatctcgtctctgctgacccagatccctgcttgcacatgtcctcgcctccagtgccctcacagagcttgccgctagacgtatccgagtcgcattcctcaaatacctcccggcaatttcttgatccaccggacagctacgactggtcgtggacctcgattggcactgacgaggctattgacactgactgctgggggctgtcccaatgtgatggaggcttcagctgtcagttagagccaacgctgccggatctaccttcgcccttcgagtctacagttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctcctatcgcaaattaggcggacccagaacagccatatgagcccactggaagggagtcgatcccagtcgccgagcagagacgacactagcagcagcagcggccacagcagtgttgacaccatacccttctttagcgagaacctccctattggtgagctgttctcctatgttgaccccctgacacacgccctattctcggcttgcactacgttacatgttggggtagaattgctgcgtgagaatgagattactctgggagtacactccgcccagggcattgcagcttccatcagcatgagcggggaaccaggcgaggatatagccaggacaggggcgaccaattccgcaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 77 - DNA, 1410 bp, Artificial Sequence
  synthetically generated variant
atggctgcagatcaaggtatattcacgaactcggtcactctctcgccagtggagggttcacacaccggtggaacattaccccgccgtgcattccgacgcgcttgtgatcggtgtcatgcacaaaagatcaaatgtactggaaataaggaggttactggccgtgctccctgtcagcgttgccagcaggctggacttcgatgcgtctacagtgagcgatgccccaagcgcaagctacgccattccagggcatcggatctcgtctctgctgacccagatccctgcttgcacatgtcctcgcctccagtgccctcacagagcttgccgctagacgtatccgagtcgcattcctcaaatacctcccggcaatttcttgatccaccggacagctacgactggtcgtggacctcgattggcactgacgaggctattgacactgactgctgggggctgtcccaatgtgatggaggcttcagctgtcagttagagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctcctgtcgcaaattaggcggacccagaacagccatatgagcccactggacgggagtcgatcccagtcgccgagcagagacgacaccagcagcagcagcggccacagcagtgttgacaccatacccttctttagcgagaacctccctattggtgagctattctcctatgttgaccccctgacacacgccctattctcggcttgcactacgttacatgttggggtacaattgctgcgtgagaatgagattactctgggagtagactccgcccagggcattgcagcttccatcagcatgagcggggaaccaggcgaggatatagccaggacaggggcgaccaattccgcaagatgcgaggagcagccgaccactccagcggctcgggttttgttcatgttcttgagtgatgaaggggctttccaggaggcaaagtctgctggttcccgaggtcgaaccatcacagtactgcgacgaagctatgaggatatcttttccctcgcccgcaaacacaaacatggcatgctcagagacctcaacaatattccttcatga

SEQ ID NO: 78 - DNA, 1410 bp, Artificial Sequence
  synthetically generated variant
atggctgcagatcaaggtatattcacgaactcggtcactctctcgccagtggagggttcacgcaccggtggaacattaccccgccgtgcactccgacgctcttgtgatcggtgtcatgcacaaaagatcaaatgtactggaaataaggaggttactggccgtgctccctgtcagcgttgccagcaggctggacttcgatgcgtctacagtgagcgatgccccaagcgcaagctacgccaatccagggcagcggatctcgtctctgctgacccagatccctgcttgcacatgtcctcgcctccagtgccctcacagagcttgccgctagacgtatccgagtcgcattcctcaaatacctcccggcaatttcttgatccaccggacagctacgactggtcgtggacctcgattggcactgacgaggctattgacactgactgctgggggctgtcccaatgtgatggaggcttcagctgtcagttagagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctcctgtcgcaaattaggcggacccagaacagccatatgagcccactggaagggagtcgatcccagtcgccgagcagagacgacaccagcagcagcagcggccacagcagtgttgacaccatacccttctttagcgagaacctccctattggtgagctgttctcctatgttgaccccctgacacacgccctattctcggcttgcactacgttacatgttggggtacaattgctgcgtgagaatgagattactctgggagtacactccgcccagggcattgcagcttccatcagcatgagcggggaaccaggcgaggatatagccaggacaggggcgaccaattccgcaagatgcgaggagcagccgatcactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 79 - DNA, 1410 bp, Artificial Sequence
  synthetically generated variant
atggctgcagatcaaggtatattcacgaactcggtcactctctcgccagtggagggttcacgcaccggtggaacattaccccgccgtgcactccgacgctcttgtgatcggtgtcatgcacaaaagatcaaatgtactggaaataaggaggttactggccgtgctccctgtcagcgttgccagcaggctggacttcgatgcgtctacagtgagcgatgccccaagcgcaagctacgccaatccagggcagcggatctcgtctctgctgacccagatccctgcttgcacatatcctcgcctccagtgccctcacagagcttaccgctagacgtatccgattcgcattcctcaaatacctcccggcaatttcttgatccaccggacagctacgactggtcgtggacctcgattggcactgacgaggctattgacactaactgctgggggctgtcccaatgtgatggaggcttcagctgtcagttagagtcaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggcctaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctcctgtcgcaaattaggcggacccagaacagccatatgagcccactggaagggagtcgatcccagtcgccgagcagagacgacaccagcagcagcagcggccacagcagtgttgacaccatacccttctttagcgagaacctccctattggtgagctgttctcctatgttgaccccctgacacacgccctattctcggcttgcactacgttacatgttggggtacaattgctgcgtgagattgagattactctgggagtacactccgcccagggcattgcagcttccatcagcatgagcggggaaccaggcgaggatatagccaggacaggggcaaccaattccgcaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaatatggcatgctcagagacctcaacaatattcctccatga

SEQ ID NO: 80 - DNA, 1410 bp, Artificial Sequence
  synthetically generated variant
atggctgcagatcaaggtatattcacgaactcggtcactctctcgccagtggagggttcacgcaccggtggaacattaccccgccgtgcattccgacgctcttgtgatcggtgtcatgcacaaaaggtcaaatgtactggaaataaggaggttactggccgtgctccctgtcagcgttgccagcaggctggacttcgatgcgtctacagtgagcgatgccccaagcgcaagctacgccaatccagggcagcggatctcgtctctgctgacccagatccctgcttgcacatgtcctcgcctccagtgccctcacagagcttgccgctagacgtatccgagtcgcattcctcaaatacctcccggcaatttcttgatccaccggacagctacgactggtcgtggacctcgattggcactgacgaggctattgacactgactgctgggggctgtcccaatgtgatggaggcttcagctgtcagttagagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctcctgtcgcaaattaggcggaccctgaacagccatatgagcccactggaagggagtcgatcccagtcgccgagcagagacgacaccagcagcagcagcggccacagcagtgttgacaccatacccttctttagcgagaacctccctattggtgagctgttctcctatgttgaccccctgacacacgccctattctcggcttgcactacgttacatgttggggtacaattgctgcgtgagaatgagattactctgggagtacactccgcccagggcattgcagcttccatcagcatgagcggggaaccaggcgaggatatagccaggacaggggcgaccaattccgcaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 81 - DNA, 1410 bp, Artificial Sequence
  synthetically generated variant
atggctgcagatcaaggtatattcacgaactccgtcactctctcgccagtggagggttcacgcaccggtggaacattaccccgccgtgcattacgacgctcttgtgatcggtgtcatgcacaaaagatcaaatgtactggaaataaggaggttactggccgtgctccctgtcagcgttgccagcaggctggacttcgatgcgtctacagtgagcgatgccccaagcgcaagttacgccaatccagggcagcggatctcgtctctgctgacccagatccctgcttgcacatgtcctcgccttcagtgccctcacagagcttgccgctagacgtatccgagtcgcattcctcaaatacctcccggcaatttcttgatccaccggacagctacgactggtcgtggacctcgattggcactgacgaggctattgacactgactgctgggggctgtcccaacgtgatggaggcttcagctctcagttaaagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctactgtcgcaaattaggctgacccagaacagccatatgagcccactggaagggagtcgatcccagtcgccgaacagagacgacaccagcagcagcagcggccacagcagtgttgacaccatacccttctttagcgagaacctccctattggtgagctgttctcctatgttgaccccctgacacacgccctattctcggcttgcactacgttacatgttggggtacaattgctgcgtgagaatgagattactctgggagtacactccgcccagggcattgcagcttccatcagcatgagcggggaaccaggcgaggatatagccaggacaggggcgaccaattccgcaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 82 - DNA, 1410 bp, Artificial Sequence
  synthetically generated variant
atggctgcagatcaaggtatattcactaactcggtcactatctcgccagtggtgggttcacgcaccggtggaacattaccccgccgtgcattccgacgctcttgtgatcggtgtcatgcacaaaagatcaaatgtactggaaataaggaggttactggccgtgctccctgtcagcgttgccagcaggctggacttcgatgcgtctacagtgagcgatgccccaagcgcaagctacgccaatccagggcagcggatctcgtctctgctgacccagatccctgcttgcacatgtcctcgcctccagtgccctcacagagtttgccgctagacgtatccgagtcgcattcctcaaatacctcccggcaatttcttgatccaccggacagctacgactggtcgtggacctcgatttgcactgacgaggctattgacactgactgctgggggctgtcccaatgtgatggaggcttcagctgtcagttagagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctcctgtcgcaaattaggcggacccagaacagccatatgagcccactggaagggagtcgatcccagtcgccgagcagagacgacaccagcagcagcagcggccacagcagtgttgacaccatacccttctttagcgagaacctccctattggtgggctgttctcctatgttgaccccctgacacacgccctattctcggcttgcactacgttacatgttgggctacaattgctgcgtgagaatgagattactctgggagtacactccgcccagggcattgcagcttccatcagcatgagcggggaatcaggcgaggatatagccaggacaggggcgaccagttccgcaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 83 - DNA, 1410 bp, Artificial Sequence
  synthetically generated variant
atggctgcagatcaaggtatattcacgaactcggtcactctctcgccagtggagggttcacgcaccggtggaacattaccccgccgtgcattccgacgctcttgtgatcggtgtcatgcacgaaagatcaaatgtactggaaataaggaggttactggccgtgctccctgtcagcgttgccagcaggctggacttcgatgcgtctacagtgagcgatgccccaagcgcaagctacgccaatccagggcagcggatctcgtctctgctgacccagatccctgcttgcacatgtcctcgcctccagtgccctcacagagcttgccgctagacgtatccgagtcgcattcctcaaatacctcccggcaatttcttgatccaccggacagctacgactggtcgtggacctcgattggcactgacgaggctattgacactgactgctgggggctgtcccaatgtgatggaggcttcagctgtcagttagagccaacgctgccggatctaccttcgcccttcgagtatacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctcctgtcgcaaattaggcggacccagaacagccatatgagcccactggaagggagtcgatcccagtcgccgagcagagacgacaccagcagcagcagcggccacagcagtgttgacaccatacccttctttagcgagaacctccctattggtgagctgttctcctatgttgaccccctgacacacgccctattctcggcttgcactacgttacatgttggggtacaattgctgcgtgagaatgagattactctgggagtacactccgcccagggcattgcagcttccatcagcatgagcggggaaccaggcgaggatatagccaggacaggggcgaccaattccacaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 84 - DNA, 1410 bp, Artificial Sequence
  synthetically generated variant
atggctgcagatcaaggtatattcacgaactcggtcactctctcgccagtggagggttcacgcaccggtggaacattaccccgccgtgcattgcgacgctcttgtgatcggtgtcatgcacaaaagatcaaatgtactggaaataaggaggttattggccgtgctccctgtcagcgttgccagcaggctggacttcgatgcgtatacagtgagcgatgccccaagcgcaagctacgccaatccagggcagcggatctcgtctctgctgacccagatccctgcttgcacatgtcctcgcctcaagtgccctcacagagcttgtcgctagacatatccgagtcgcattcctcaaatacctcccggcaatttcttgatccaccggacagctacgactggtcgtggacctcgattggcactgacgaggctattgacactgactgctgggggctgtcccaatgtgatggaggcttcagctgtcagttagagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctcctgtcgcaaattaggcggacccagaacagccatatgagcccactggaagggagtcgatcccagtcgccgagcagagacgacaccagcagcagcagcggccacagcagtgttgacaccatacccttctttagcgagaacctccctattggtgagctgttctcctatgttgaccccctgacacacgccctattctcggcttgcactacgttacatgttggggtacaattgctgcgtgagaatgagattactctgggagtacactccgcccagggcattgcagcttccatcagcatgagcggggaaccaggcgaggatatagccaggacaggggcgaccaattccgcaagatgcgaggagcagccgaccactccagcggctcgggttttgttcatgttcttgagtgatgaaggggcattccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 85 - DNA, 1410 bp, Artificial Sequence
  synthetically generated variant
atggctgcagatcaaggtatattcacgaactcggtcactctctcaccagtggagggttcacgcaccggtggaacattaccccgccgtgcattccgacgctcttgtgatcggtgtcatgcacaaaagatcaaatgtactggaaataaggaggttactggccgtgctccctgtcagcgttgccagcaggctggacttcgatgcgtctacagtgagcgatgccccaagcgcaagctacgccaatccagggcagcgaatctcgtctctgctgacccagatccctgcttacacatgtcctcgcctccagtgccctcacagagcttgccgctagacgtatccgagtcgcattcctcaaatacctcccggcaatttcttgatccaccggacagctacgactggtcgtggacctcgattggcactgacgaggcttttgacactgactgctgggggctatcccaatgtgatggaggcttcagctgtcagctagagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatctttttcaatgcgtcacgacggcttcttactgtcctgcgccagcaagcgcaggccgactgccatcaaggcacactagacgaatgtttacggaccaagaacctctttacggcagtacactgttacatattgaatgtgcggattttgaccgccatatcggagttgctcctgtcgcaaattaggcggacccagaacagccatatgagcccactggaagggagtcgatcccagtcgccgagcagagacgacatcagcagcagcagcggccacagcagtgttgacaccatacccttctttagcgagaacctccctattggtgagctgttctcctatgttgaccccctgacacacgccctattctcggcttgcactacgttacatgttggggtacaattgctgcgtgagaatgagattactctgggagtacactccgcccagggcattgcagcttacatcagcaagagcggggaaccaggcgaggatatagccaggacaggggcgaccaattccgcaagatgcgaggagcagccgaccactccagcggctcgggtgttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 86 - DNA, 1410 bp, Artificial Sequence
  synthetically generated variant
atggctgcagatcaaggtatattcacgaactcggtcactctctcgccagtggagggttcacgcaccggtggaacattaccccgccgtgcattccgacgctcttgtgatcggtgtcatgcacaaaagatcaaatgtattggaaataaggaggttactggccgtgctccctgtcagcgttgccaacgggctggacttcgatgcgtctacagtgagcgatgccccaagcgcaggctacgccaatccagggcagcggatctcgtctctgctgacccagatccctgcttgcacatgtcctcgcctccagtgccctcacagagcttgccgctagacgtatccgagtcgcattcctcaaatacctcccggcaatttcttgatccaccggacagctacgactggtcgtggacctcgattggcactgacgaggctattgacactgactgctgggggctgtcccaatgtgatggaggcttcagctgtcagttagagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcttcacgacggcttcttactgtcctgcgccaacaagctcaggccgactgccatcaaggcacactagacgaatgtttacggaccaagaacctctttacggcagtacactgttacatattgaatgtgcggattttgaccgccatatcggagttgctcctgtcgcaaattaggcggacccagaacagccatatgagcccactggaagggagtcgatcccagtcgccgagcagagacgacaccagcagcagcagcggccacagctgtgtcgacaccatacccttctttagcgagaacctccctattggtgagctgttctcctatgttgaccccctgacacacgccctattctcggcttgcactacgttacatgttggggtacaattgctgcgtgagtatgagattactctgggaatacactccgcccagggcattgcagcttccatcagcatgagcggggaaccaggcgaggatatagccaggacaggggcgaccaattccgcaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagatctcaacaatattcctccatga

SEQ ID NO: 87 - DNA, 1410 bp, Artificial Sequence
  synthetically generated variant
atggctgcagatcaaggtatattcacgaactcggtcactctctcgccagtggagggttcacgcaccggtggaacattaccccgccgtgcattccgacgctcttgtgatcggtgtcatgcacgaaagatcaaatgtactggaaataaggaggttactggccgtgctccctgtcagcgttgccagcaagctggacttcgatgcgtctatagtgagcgatgccccaagcgcaagctacgccaatccagggcagcggatctcgtctctgctgacccagatccctgcttgcacatgtcctcgcctccagtgccctcacagagcttgccgctagacgtatccgagtcgcattcctcaaatacctcccggcaatttcttgatccaccggacagctacgactggtcgtggacctcgattggcactgacgaggctattgacactgactgctgggggctgtcccaatgtgatggaggcttcagctgtcagctagagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctcctgtcgcaaattaggcggatccagaacagccatatgagcccactggaagggagtcgatcccagtcgctgagcagagacgacaccagcagcagtagcggccacagcagtgttgacaccatacccttctttagcgagaacctccctattgatgagctgttctcctatgttgaccccctgacacacgccctattctcggcttgcactacgttacatgttggggtacaattgctgcgtgagaatgagattactctgggagtacactccgcccagggcattgcagcttccatcagcatgagcggggaactaggcgaggatatagtcaggacaggggcgaccaattccgcaagatgcgaggagcagccgaccactccagcggctcgggttttgttcatgttcttgagtgatgaaggggctttccaggaggcaaagtctgctggttcccgaagtcgaaccatcgcagcactgcgacgatgctatgaggatatcttttccctcgcccgcaaacacaaacatggcatgctcagagacctcaacaatattcctccatga

SEQ ID NO: 88 - DNA, 1410 bp, Artificial Sequence
  synthetically generated variant
atggctgcagatcaaggtatattcacgaactcggtcactctctcaccagtggagggttcacgcaccggtggaacattaccccgccgtgcattccgacgctcttgtgatcggtgtcatgcacaaaagatcaaatgtactggaaataaggaggttaatggccgtgctccctgtcagcgttgccagcaggctggacttcgatgcgtctacagtgagcgatgccccaagcgcaagctacgccaatccagggcagcggatctcgtctctgctgacccagatccctgcttgcacatgtcctcgcctccagtgccctcccagagcttgccgctagacatatccgagtcgcattcctcaaatacctcccggcaatttcttgatccaccggacagctacgactggtcgtggacctcgattggcattgacgaggctattgacactgactgctgggggctgtcccaatgtgatggaggcttcagctgtcagttagagccaacgctgccggatctaccttcgcccttcgagtctacggttgaaaaagctccgttgccaccgatatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttggccgccatatcggagttgctcctgtcgcaaattaggcggacccagaacagccatatgagcccactggaagggagtcgatcccagtcgccgagcagagacgacaccagcagcagcagcggccacagcagtgttgacaccatacccttctttagcgagaacctccctattggtgagctgttctcctatgttgaccccctgacacacgccctattctcggcttgcactacgttacatgttggggtacaattgctgcgtgagaatgagattactctgggagtacactccgcccagggcattgcagcttccatcagcatgagcggggaaccaggcgaggatatagccaggacaggggcgaccaattccgcaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 89 - DNA, 1410 bp, Artificial Sequence
  synthetically generated variant
atggctgcagaacaaggtatattcacgaactcggtcactctctcgccagtggagggttcacgcaccggtggaacattaccccgccgtgcattccgacgctcttgtgatcggtgtcatgcacgaaagatcaaatgtactggaaataaggaggttactggccgtgctccctgtcagcgttgccagcaggctggacttcgatgtgtctacagtgagcgatgccccaagcgcaagctacgccaatccagggcagcggatctcatctctgctgacccagatccctgcttgcacatgtcctcgcctccagtgccctcacagagcttgccgctagaagtatccgagtcgcattcctcaaatacctcccggcaatttcttgatccaccggacagctacgactggtcgtggacctcgattggcactgacaaggctattgacactgactgctgggggctgtcccaatgtgatggaggcttcagctgtcagttagagccaacgctgccggatctaccttcgccctttgagtctacggttgaaaaagctccgttgccaccggtatcgagcgacattactcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattggatgtgcggattttgaccgccatatcggagttgctcctgtcgcaaattaggcggacccagaacagccatatgagcccactggaagggagtcgatcccagtcgccgagcagagacgacaccagcagcagcagcggccacagcagtgttgacaccatacccttctttagcgagaacctccctattggtgagctgttctcctatgttgaccccctgagacacgccctattctcggcttgcactacgttacatgttggggtacaattgctgcgtgagattgagattactctgggagtacactccgcccggggcattgcagcttccatcagcatgagcggggaaccaggcgaggatatagccaggacaggggcgaccaattccgcaagatgcgaggagcagccgaccactccagcggctcgggttttgttcatgttcttgagtgatgaagggactttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 90 - DNA, 1410 bp, Artificial Sequence
  synthetically generated variant
atggctgcagatcaaggtatattcacgaactcggtcactctctcgccagtggagggttcacgcaccggtggaacattaccccgccgtgcattccgacgctcttgtgatcggtgtcatgcacaaaagatcaaatgtactggaaataaggaggttactggccgtgctccctgtcagcgttgccagcaggctggacttcgatgcgtctacagtgagcgatgccccaagcgcaagctacgccaatccagggcagcggatctcgtctctgctgacccagatccctgcttgcacatgtcctcacctccagtgccctcacagagcttgccgctagacgtatccgagtcgcattcctcaaatacctcccggcaatttcttgatccaccggacagctacaactggttgtggacctcgattggcactgacgaggctattgacactgactgctgggggctgtcccaatgtgatggaggcttcagctgtcagttagagccaacgctgccggatctaccttcgcccttcgaatctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctcctgtcgcaaattaggcggacccagaacagccatatgagcccactggaagggagtcgatcccagtcgccgagcggagacgacaccagcagcagcagcggccacagcagtgttgacaccatacccttctttagcgagaacctccctattggtgagctgttctcctatgttgaccccctgacacacgccctattctcggcttgcactacgttacatgttggggtacaattgctgcgtgagaatgagattactctgggagtacactccgcccagggtattgcagcttccatcagcatgagcggggaaccaggcgaggatatagccaggacaggggcgaccaattccgcaagatgcgaggagcagccgaccactccagcggctcgggttttgttcatgttcttgagtgatgaaggggctttccaggagggaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 91 - protein, 469 aa, Aspergillus terreus
MAADQGIFTNSVTLSPVEGSRTGGTLPRRAFRRSCDRCHAQKIKCTGNKEVTGRAPCQRCQQAGLRCVYSERCPKRKLRQSRAADLVSADPDPCLHMSSPPVPSQSLPLDVSESHSSNTSRQFLDPPDSYDWSWTSIGTDEAIDTDCWGLSQCDGGFSCQLEPTLPDLPSPFESTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRTQNSHMSPLEGSRSQSPSRDDTSSSSGHSSVDTIPFFSENLPIGELFSYVDPLTHALFSACTTLHVGVQLLRENEITLGVHSAQGIAASISMSGEPGEDIARTGATNSARCEEQPTTPAARVLFMFLSDEGAFQEAKSAGSRGRTIAALRRCYEDIFSLARKHKHGMLRDLNNIPP

SEQ ID NO: 92 - DNA, 1410 bp, Aspergillus terreus
atggctgcagatcaaggtatattcacgaactcggtcactctctcgccagtggagggttcacgcaccggtggaacattaccccgccgtgcattccgacgctcttgtgatcggtgtcatgcacaaaagatcaaatgtactggaaataaggaggttactggccgtgctccctgtcagcgttgccagcaggctggacttcgatgcgtctacagtgagcgatgccccaagcgcaagctacgccaatccagggcagcggatctcgtctctgctgacccagatccctgcttgcacatgtcctcgcctccagtgccctcacagagcttgccgctagacgtatccgagtcgcattcctcaaatacctcccggcaatttcttgatccaccggacagctacgactggtcgtggacctcgattggcactgacgaggctattgacactgactgctgggggctgtcccaatgtgatggaggcttcagctgtcagttagagccaacgctgccggatctaccttcgcccttcgagtctacggttgaaaaagctccgttgccaccggtatcgagcgacattgctcgtgcggccagtgcgcaacgagagcttttcgatgacctgtcggcggtgtcgcaggaactggaagagatccttctggccgtgacggtagaatggccgaagcaggaaatctggacccatcccatcggaatgtttttcaatgcgtcacgacggcttcttactgtcctgcgccaacaagcgcaggccgactgccatcaaggcacactagacgaatgtttacggaccaagaacctctttacggcagtacactgttacatattgaatgtgcggattttgaccgccatatcggagttgctcctgtcgcaaattaggcggacccagaacagccatatgagcccactggaagggagtcgatcccagtcgccgagcagagacgacaccagcagcagcagcggccacagcagtgttgacaccatacccttctttagcgagaacctccctattggtgagctgttctcctatgttgaccccctgacacacgccctattctcggcttgcactacgttacatgttggggtacaattgctgcgtgagaatgagattactctgggagtacactccgcccagggcattgcagcttccatcagcatgagcggggaaccaggcgaggatatagccaggacaggggcgaccaattccgcaagatgcgaggagcagccgaccactccagcggctcgggttttgttcatgttcttgagtgatgaaggggctttccaggaggcaaagtctgctggttcccgaggtcgaaccatcgcagcactgcgacgatgctatgaggatatcttttccctcgcccgcaaacacaaacatggcatgctcagagacctcaacaatattcctccatga

SEQ ID NO: 93 - protein, 480 aa, Artificial Sequence
  Synthetically generated variant
MTQDTAQYRGAMAADQGIFTNSVTLSPVEGSRTGGTLPRRAFRRSCDRCHAQKIKCTGNKEVTGRAPCQRCQQAGLRCVYSERCPKRKLRQSRAADLVSADPDPCLHMSSPPVPSQSLPLDVSESHSSNTSRQFLDPPDSYDWSWTSIGTDEAIDTDCWGLSQCDGGFSCQLEPTLPDLPSPFESTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRTQNSHMSPLEGSRSQSPSRDDTSSSSGHSSVDTIPFFSENLPIGELFSYVDPLTHALFSACTTLHVGVQLLRENEITLGVHSAQGIAASISMSGEPGEDIARTGATNSARCEEQPTTPAARVLFMFLSDEGAFQEAKSAGSRGRTIAALRRCYEDIFSLARKHKHGMLRDLNNIPP

SEQ ID NO: 94 - protein, 482 aa, Artificial Sequence
  Synthetically generated variant
MLMTQDTAQYRGAMAADQGIFTNSVTLSPVEGSRTGGTLPRRAFRRSCDRCHAQKIKCTGNKEVTGRAPCQRCQQAGLRCVYSERCPKRKLRQSRAADLVSADPDPCLHMSSPPVPSQSLPLDVSESHSSNTSRQFLDPPDSYDWSWTSIGTDEAIDTDCWGLSQCDGGFSCQLEPTLPDLPSPFESTVEKAPLPPVSSDIARAASAQRELFDDLSAVSQELEEILLAVTVEWPKQEIWTHPIGMFFNASRRLLTVLRQQAQADCHQGTLDECLRTKNLFTAVHCYILNVRILTAISELLLSQIRRTQNSHMSPLEGSRSQSPSRDDTSSSSGHSSVDTIPFFSENLPIGELFSYVDPLTHALFSACTTLHVGVQLLRENEITLGVHSAQGIAASISMSGEPGEDIARTGATNSARCEEQPTTPAARVLFMFLSDEGAFQEAKSAGSRGRTIAALRRCYEDIFSLARKHKHGMLRDLNNIPP

SEQ ID NO: 95 - protein, 11 aa, Aspergillus terreus
MTQDTAQYRGA

SEQ ID NO: 96 - protein, 13 aa, Aspergillus terreus
MLMTQDTAQYRGA